(19) United States
(12) Patent Application Publication
(10) Pub. No.: US 2026/0167972 A1
(43) Pub. Date: Jun. 18, 2026

(54) AMINO ACID DERIVATIVES AS NOVEL DELIVERY AGENTS FOR FUNCTIONAL DELIVERY OF OLIGONUCLEOTIDES

(71) Applicant: University of Rochester, Rochester, NY (US)

(72) Inventors: Bradley L. Nilsson, Rochester, NY (US); Melissa L. Jagrosse, Northford, CT (US); Pamela A. Sanin, Richardson, TX (US)

(73) Assignee: University of Rochester, Rochester, NY (US)

(21) Appl. No.: 19/127,012

(22) PCT Filed: Nov. 3, 2023
(86) PCT No.: PCT/US2023/078628

§ 371 Date: May 2, 2025

Related U.S. Application Data
(60) Provisional application No. 63/422,967, filed on Nov. 5, 2022.

(51) Int. Cl. C12N 15/113; A61K 9/00; C12N 15/11

(52) U.S. Cl. CPC C12N 15/1138; A61K 9/0019; C12N 15/111; C12N 15/113; C12N 2310/14; C12N 2310/321

(57) ABSTRACT

A nanoparticle complex contains short-interfering RNA (siRNA) and one or more amino acid derivatives. The nanoparticles may be spherical or micelle-like. The complex may have a mixture of spherical/micelle-like and fibril-like nanoparticles, or a mixture of spherical/micelle-like, sheet-like and/or fibril-like nanoparticles. The complexes may be used as part of a method of delivering RNA or DNA into a cell, where the RNA or DNA is mixed with one or more amino acid derivatives, the mixture is diluted into a cell culture medium, and a cell is dosed with the diluted mixture. The RNA may be small interfering RNA (siRNA). The method may reduce messenger RNA (mRNA) levels and/or protein levels related to a specific gene or genes by at least 50% and exhibit cell viabilities equal to or exceeding 40% after 48 hours.

### CROSS-REFERENCE TO RELATED APPLICATIONS

This application claims priority to and the benefit of co-pending U.S. provisional patent application Ser. No. 63/422,967, entitled AMINO ACID DERIVATIVES AS NOVEL DELIVERY AGENTS FOR FUNCTIONAL DELIVERY OF OLIGONUCLEOTIDES, filed Nov. 5, 2022, which is incorporated herein by reference in its entirety.

### STATEMENT REGARDING FEDERALLY SPONSORED RESEARCH OR DEVELOPMENT

This invention was made with government support under HL138538 awarded by the National Institutes of Health and W81XWH-20-1-0112 awarded by the ARMY Medical Research and Materiel Command. The government has certain rights in the invention.

### I. TECHNICAL FIELD

The present invention generally relates to delivery systems for a wide range of compounds including small molecules, oligonucleotides, vaccines and proteins. Specifically, the invention relates to delivery systems for oligonucleotides including antisense oligonucleotides (ASOs), such as gapmers, small interfering RNAs (siRNAs), short-hairpin RNA (shRNA), micro-RNA (miRNA), large RNAs, such as messenger RNA (mRNA) and DNA, such as plasmid DNA (pDNA).

### II. BACKGROUND

Gene therapy is a rapidly growing field that utilizes oligonucleotides to treat or prevent disease by editing faulty genes, introducing new ones, or modifying the expression of disease-related proteins. Although the field of gene therapy was first conceptualized in 1972, Waclaw Szybalski demonstrated that exogenous DNA could result in rescue of heritable enzymatic deficiencies in mammalian cells in 1962. In the 70 years since Szybalski's initial discovery, a variety of genome editing techniques have been developed including the engineering of guided endonucleases, such as transcription activator-like effector nucleases (TALENs) and zinc finger nucleases (ZFNs). Additionally, endogenous cellular pathways have been manipulated for gene therapy applications, such as CRISPR-Cas9 (gene insertion) and RNA interference (RNAi; gene silencing).

First discovered in C. elegans in 1998, RNAi is an endogenous pathway that induces gene silencing by utilizing information stored in exogenous or endogenous double-stranded RNAs (dsRNA). Exogenous non-coding dsRNA is processed into short-interfering RNA (siRNA) by the endoribonuclease, Dicer. The RNA-induced silencing complex (RISC) is then formed by complexation of the argonaut 2 protein (Ago2) with the siRNA-Dicer complex, where the double-stranded siRNA is unwound, the passenger strand is degraded, and the remaining guide strand is used for target mRNA recognition through complementary binding (FIG. 1). The cellular RNAi machinery utilizes siRNA to induce gene silencing with high target specificity and rapid, efficient, and short-term degradation of mRNA(s). Upon cellular entry, exogeneous dsRNA is processed by Dicer and Ago2 to form the RISC. The guide strand directs the RISC to the targeted mRNA to silence gene expression by heterochromatin formation, mRNA degradation, or inhibition of transcription or translation. Endogenous miRNAs and shRNAs transcribed in the nucleus are processed by Drosha/DGCR prior to nuclear export by exportin-5 (XPO5). Once in the cytosol, the processed miRNA/shRNA can be incorporated into the RISC to induce genetic silencing.

Short-hairpin RNA (shRNA) and micro-RNA (miRNA) are endogenous RNAs that operate by similar mechanisms. Both RNAs are transcribed in the nucleus; miRNAs are processed by RNA polymerase II, while shRNAs are processed by either RNA polymerase II or RNA polymerase III, with their primary transcript containing similar stem-loop hairpin structures. The primary transcripts are processed by a ribonuclease III enzyme called Drosha and the heme-bound DGCR protein. The partially processed RNAs can then be transported to the cytoplasm by XPO5, where they are then cleaved and processed by Dicer and the RISC in a similar manner as siRNA. Upon sequence recognition, Ago2 binds the target mRNA to inhibit translational machinery or signal for degradation, depending on siRNA-mRNA mismatching, and occasionally promoting heterochromatin formation, resulting in long-term or permanent gene silencing.

RNAi is a useful tool for studying a variety of genetic disorders because many diseases are caused by aberrant protein expression or the expression of proteins that are modified to alter normal function. The ability to modify gene expression by the administration of exogenous agents thus has great potential for the treatment of these types of disorders. For example, silencing of anti-apoptosis genes and inhibition of tumor angiogenesis related factors provides a new avenue for patient treatment in oncology. One class of potential targets are miRNAs, which are involved in cell proliferation, differentiation, cell cycle regulation, and apoptosis, and have been aberrantly expressed during various stages of cancer development, metastasis, and cancer stem cell regulation. miR-21 is one of the most frequently upregulated miRNAs in cancer and acts by limiting the activity of signaling pathways associated with increased tumor cell survival, including PTEN, tropomyosin I, PDCD4, RECK and TIMP, and SPRY2. Transfection of 2′-O-methyl-antisense oligonucleotides and LNA-DNA antisense oligonucleotides inhibits miR-21 in glioblastoma and breast cancer cells in vitro as a result of increased caspase-mediated apoptosis, in addition to reducing tumor invasion in MDA-MB-231 cells, and RKO colon cancer cells. Administration of LNA-anti-miR-21 has also been shown to inhibit glioma growth in vivo.

Cholesterol-modified siRNAs have also been utilized for inhibiting other oncogenic miRNAs including, miR-16, miR-122, miR-192, and miR-194. Kinesin spindle protein (KSP) is a mitotic spindle motor protein essential for chromosome segregation during mitosis; inhibition of this KSP results in cell cycle arrest and apoptosis. Additionally, polo-like kinase I (PLK1) plays an important role in mitosis and cytokinesis, with its overexpression commonly observed in several human cancer lines. Targeting KSP and PLK1 using an RNAi strategy results in increased cell death via apoptosis both in vitro and in vivo. Combination of PLK1-siRNA with a peptide fusion protein containing HER2 scFv utilizing PEG-PLA nanoparticles demonstrates successful targeting delivery for increased cancer cell death in HER2+ breast cancer. Other potential targets for RNAi-mediated silencing are protein kinase N3 (PKN3), M2 subunit of ribonucleotide reductase (RRM2), VEGF-A, KRasg12D, Tenasion-c, and Furin.

Currently, there are five FDA-approved RNAi-therapeutics. They include Patisiran and Vutrisiran for treatment of hereditary transthyretin-mediated amyloidosis (HTTR); Givosiran, for treatment of acute hepatic porphyria; Lumasiran for treatment of primary hyperoxaluria type 1; and Inclisiran for adults with heterozygous familial hypercholesterolemia (HeFH) or clinical atherosclerotic cardiovascular disease (ASCVD). Unfortunately, broad clinical application of RNAi has been impeded by technical challenges, including barriers to cytosolic delivery of siRNA oligonucleotide. The major obstacle for in vivo RNAi therapy is efficient siRNA delivery to the cell in a form that is accessible to the RNAi machinery.

Cellular uptake of large biomolecules is predominantly governed by endocytosis, in which large molecules are transported across the cellular membrane via endocytic vesicles. Endocytosis can be divided into two main categories; phagocytosis and pinocytosis. Pinocytosis can be further sub-divided into macropinocytosis, clathrin/caveolin-dependent, and clathrin/caveolin-independent pathways (FIG. 2). The most common type of cellular entry observed for macromolecules is clathrin-dependent endocytosis. This pathway involves the budding of endosomes encapsulating the siRNA from the cellular membrane and fusing with lysosomes commonly resulting in degradation of the siRNA payload. Therefore, cellular uptake of therapeutic molecules via endocytic pathways requires a delivery system that enables early endosomal escape. Oligonucleotides show low immune and inflammatory responses in vivo; however, they are poorly transported across the cell membrane and subject to competitive degradation by endogenous nucleases. Naked siRNA transduction occurs effectively only with the use of electroporation, sonoporation, jet injection, or related membrane disruption methods. For some disease applications, critical considerations in dosing schedules must be made, as dilution of siRNA due to rapid cell proliferation may alter the duration of gene silencing effects.

An ideal delivery system should be biodegradable, biocompatible, non-cytotoxic, non-immunogenic, and should adequately protect the payload from premature degradation. In addition, the ideal delivery agent will facilitate translocation of siRNA cargo into cells without damaging the cell membrane or other cellular components and must bind siRNA reversibly to allow for selective release of the RNA from the carrier in the intracellular environment. Numerous siRNA delivery systems have been explored, including viral vectors, lipid-based carriers, cationic polymers, antibody constructs, and RNA aptamers. Although significant progress in RNAi delivery has been made with the listed transfection agents, none are ideal, and significant barriers have thus far limited clinical applications with these agents.

A summary of advantages and disadvantages of different vectors that have been utilized for siRNA delivery are set forth in Table 1 below.

TABLE 1

| Delivery Vector | | Advantages | Disadvantages |
|---|---|---|---|
| Viruses | Adenovirus | Highly efficient transduction profile | Immunological responses, |
| | | | non-specific cell targeting, |
| | | | acute |
| | AAV | Highly efficient transduction profile, | Requires helper virus, small |
| | | reduce inflammatory and immune | cloning capacity |
| | | responses | |
| | Retrovirus | High gene transduction | Dependence of cell |
| | | | differentiated state, insertion |
| | | | into host genomes |
| | Lentivirus | High gene transduction, cell | Insertion into host genomes |
| | | differentiated state independence | |
| Lipids | Cationic | Good transfection efficiency, | Poor stability, cellular toxicity, |
| | | electrostatic interactions with siRNA | immune response elicitation |
| | Neutral | Reduced cytotoxicity | Reduced ability to complex |
| | | | with siRNA |
| | Nanoparticles | Charge variable, complex formation | Synthetic challenges |
| | | prior to administration, neutralized at | |
| | | physiological pH, reduced toxicity and | |
| | | immunological responses, increased | |
| | | membrane destabilizing capacity, | |
| | | higher | |
| Polymers | Polycations | Modifiable size | Weaker interactions with siRNA |
| | Nanoparticles | Biocompatible, biodegradable, | Large surface area causes |
| | | synthetic diversity | aggregation |

Peptides as siRNA Delivery Vectors: A wide range of cell-penetrating peptides (CPPs) have been exploited for siRNA delivery. Most CPPs that have been used for siRNA delivery are cationic to improve interactions with cell-surface anionic glycoproteins and to promote beneficial electrostatic interactions with the anionic phosphate backbone of oligonucleotide cargo. Proteins and peptides containing double-stranded RNA binding domains have also been complexed with siRNA for cellular delivery. Cellular uptake of siRNA complexed with CPPs can occur via endocytic or non-endocytic pathways, depending on the particle size, peptide type, and siRNA loading method. HIV-1 trans-activator protein (TAT) has been used for delivery of p38 MAP kinase siRNA to the mouse lung via intratracheal administration. Disulfide-linked conjugation of siRNA with TAT results in 20-30% knockdown 6 hours after administration. While TAT is a commonly used CPP, some studies have shown non-specific cellular uptake with this vector. Other CPPs that can be used in siRNA delivery include Protamine 1, Penetratin-1, (PPR)ₙ, (PRR)ₙ, DPV peptides, MPG, POA, viral proteins, transportan, VP22, and MAP synthetic arginine-rich peptides. Although a number cell-penetrating peptide (CPP) systems have been validated for in vitro and in vivo siRNA delivery, clinical applications remain limited by issues with limited serum half-life, cytotoxicity, and the high cost of peptide production.

Cyclic amphipathic peptide (CAP) delivery systems: Another type of delivery system for siRNA is a cyclic amphipathic peptide (CAP) delivery system. The in vivo use of these agents has been demonstrated for delivery to the lung. The peptides in the CAP system consist of alternating hydrophobic and hydrophilic amino acids flanked by a cysteine residue at each terminus, which are then cyclized via oxidative intramolecular disulfide bond formation, making these materials resistant to degradation by proteases. These CAPs form non-covalent complexes with siRNA, that protect the complexed siRNA from degradation by nucleases, and allow for effective transport into the cytosol. Upon cellular entry, the peptide disulfide bonds are reduced, likely by excess cellular glutathione, and the linearized peptides are proteolytically degraded, resulting in release of the siRNA payload. Cyclic Ac-C(FKFE)₂CG-NH₂ and Ac-C(WR)₄CG-NH₂ peptides complexed with rhodium-labeled siRNA (Rh-siRNA) were incubated with human A549 lung adenocarincoma cells, demonstrating improved Rh-siRNA cellular delivery compared with naked Rh-siRNA. Additionally, Ac-C(WR)₄CG-NH₂ showed significantly enhanced cytosolic delivery of Rh-siRNA, compared to cyclic Ac-C(FKFE)₂CG-NH₂ and naked Rh-siRNA. Although L- and D-isomers demonstrated similar cytosolic delivery capacity, gene knockdown was significantly more successful with the L-isomer, suggesting that proteolytic degradation of the CAP is important for efficient siRNA release. In vivo studies utilizing CAPs complexed with thyroid transcription factor-1 (TTF-1) siRNA resulted in approximately 80% gene knockdown.

Supramolecular Assemblies as siRNA Delivery Vectors: An alternative delivery vehicle is supramolecular assemblies that form extensive supramolecular assemblies and hydrogels. Peptide-based materials have been utilized for encapsulation and sustained release of small molecule cancer drugs, such as doxorubicin and cisplatin. Polymer-based materials have been extensively reported for the sustained release of biomacromolecules in vitro and in vivo. Polyethyleneimine-poly(organophosphazene) polyplex hydrogels have been utilized for encapsulation and localized delivery of cyclin B1-siRNA. Incubation of PC-3 cells with cyclin B1-siRNA-loaded hydrogels resulted in approximately 80% reduction of cyclic B1 expression and significant reduction of tumors has been demonstrated in vivo. Other polymer-based hydrogels have also been utilized for delivery of oligopeptide-terminated poly(β-aminoester) (pBAE) nanoparticles encapsulating siRNA. MDA-MB-231-GFP cells exposed to pBAE nanoparticles loaded with anti-GFP siRNA demonstrated a 50% reduction in eGFP expression 48 hours post-transfection, with no reduction in cell viability. Delivery of anti-Luciferase-siRNA to mice pre-injected with luciferase-expressing MDA-MB-231 tumor cells resulted in 60% reduction of luciferase. Recently, Fliervoet et al. demonstrated thermosensitive release of siRNA from di-block poly(N-isopropylacrylamide) (PNIPAM) hydrogels, resulting in up to 70% gene knockdown in vitro.

Low-molecular-weight hydrogels: Low-molecular-weight hydrogels consisting of fluorinated phenylalanine derivatives containing an N-terminal Fmoc protecting group (Fmoc-Phe) provide another class of hydrogel materials for drug and oligonucleotide release. Cationic Fmoc-Phe derivatives have been reported that spontaneously form hydrogel networks, without the need for organic co-solvents, by modification of the N-terminus with diaminopropane (DAP). Fmoc-F₅-Phe-DAP hydrogels have been utilized for successful in vivo drug delivery. Fmoc-F₅-Phe-DAP hydrogels were pre-loaded with diclofenac prior to injection into the ankle of injured mice. After measuring mechanical sensitivity of the affected area, it was determined that the Fmoc-F₅-Phe-DAP hydrogel loaded with diclofenac provided enhanced pain relief over a maintained period of 14 days. Meanwhile, a solution of diclofenac in saline never provided the same level of pain relief and began to have a reduced effect after 11 days. The ability to expand these biomaterials to oligonucleotide delivery would provide a cost-effective alternative approach for localized gene therapy applications.

### III. SUMMARY

Disclosed is a nanoparticle complex comprising: short-interfering RNA (siRNA) and one or more amino acid derivatives of Formula I, II, III, IV, V or VI:

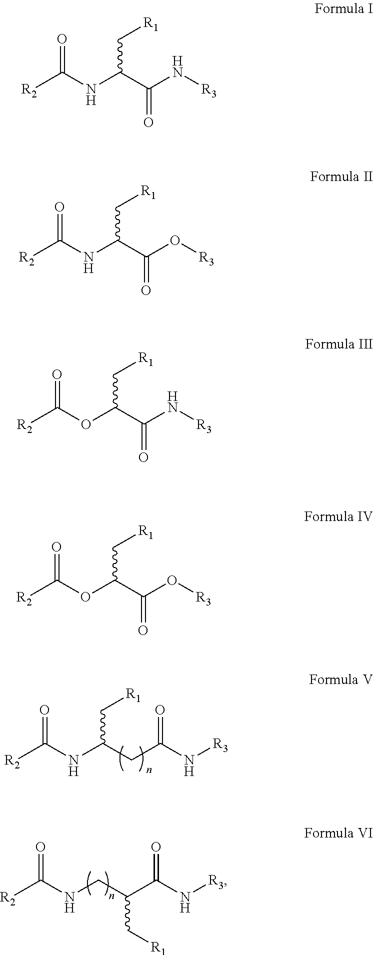

where R₁ is a canonical or non-canonical amino acid; R₂ is a hydrophobic, aromatic or charged modifying group; R₃ is hydrophobic, aromatic or charged modifying group; and n is an integer from 1 to 4. In one embodiment, R₂ is selected from the group consisting of:

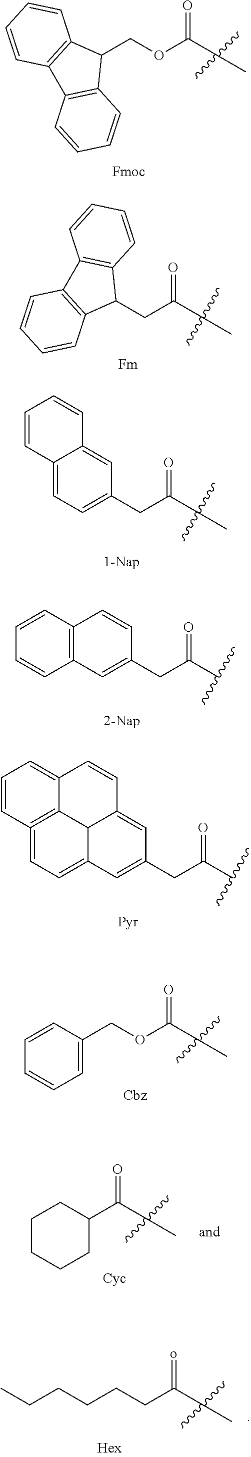

In another embodiment, R₃ is selected from the group consisting of:

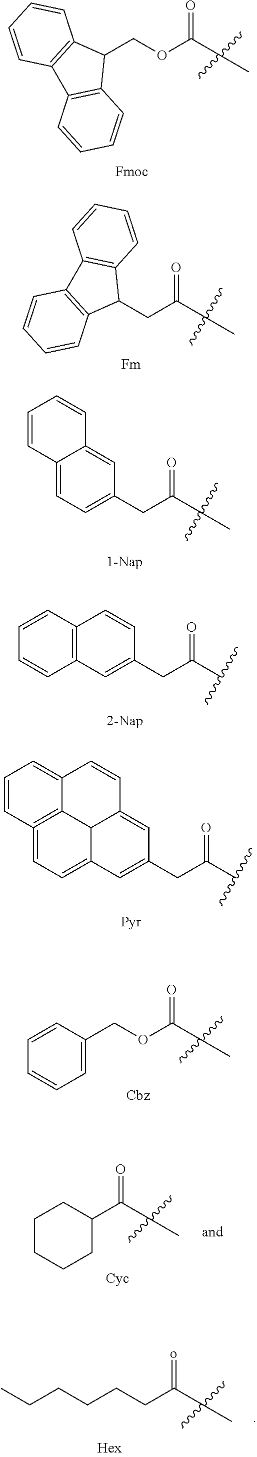

In yet another embodiment, R₂ and R₃ are each selected from the group consisting of:

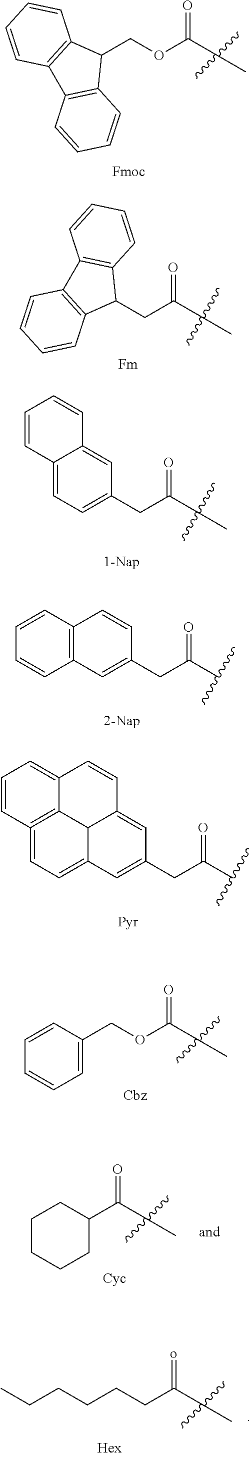

In another embodiment, R₂ is selected from the group consisting of:

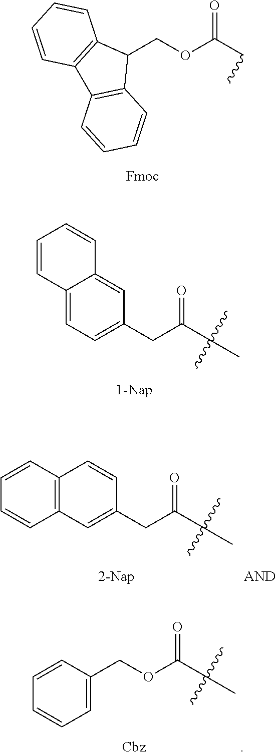

In another embodiment, R₃ is selected from the group consisting of:

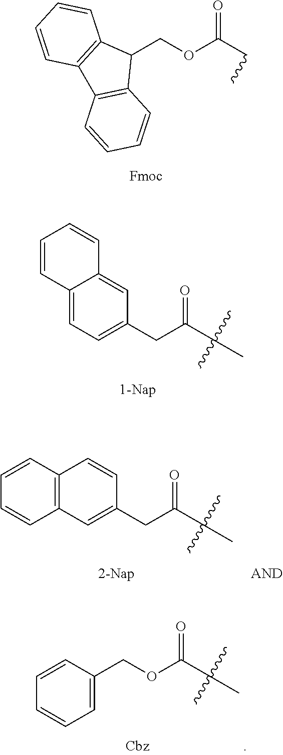

In yet another embodiment, R₂ and R₃ are each selected from the group consisting of:

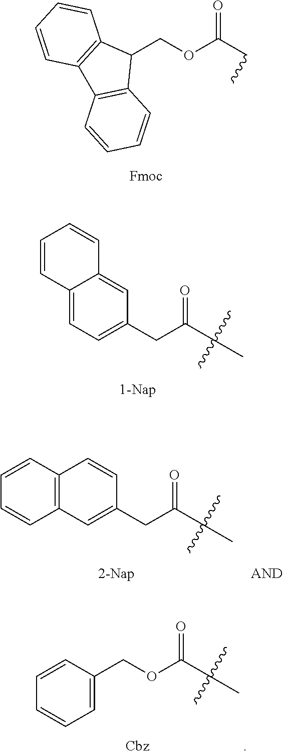

The disclosed nanoparticle complex may contain more than one amino acid derivative. In different embodiments, the disclosed nanoparticle complex may contain spherical or micelle-like nanoparticles, a mixture of spherical/micelle-like and fibril-like nanoparticles, or a mixture of spherical/micelle-like, sheet-like and/or fibril-like nanoparticles.

Another embodiment is a nanoparticle complex comprising: short-interfering RNA (siRNA) and one or more phenylalanine derivatives with the formula:

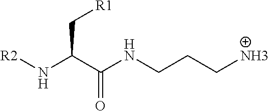

where R₁ is benzene or a fluorinated benzene; and R₂ is an N-terminal modifying group. In one preferred embodiment, R₁ is selected from the group consisting of:

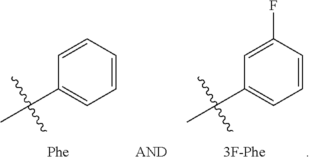 [SMILES: *c1ccccc1 ; *c1cccc(F)c1]

In another preferred embodiment, R₂ is selected from the group consisting of:

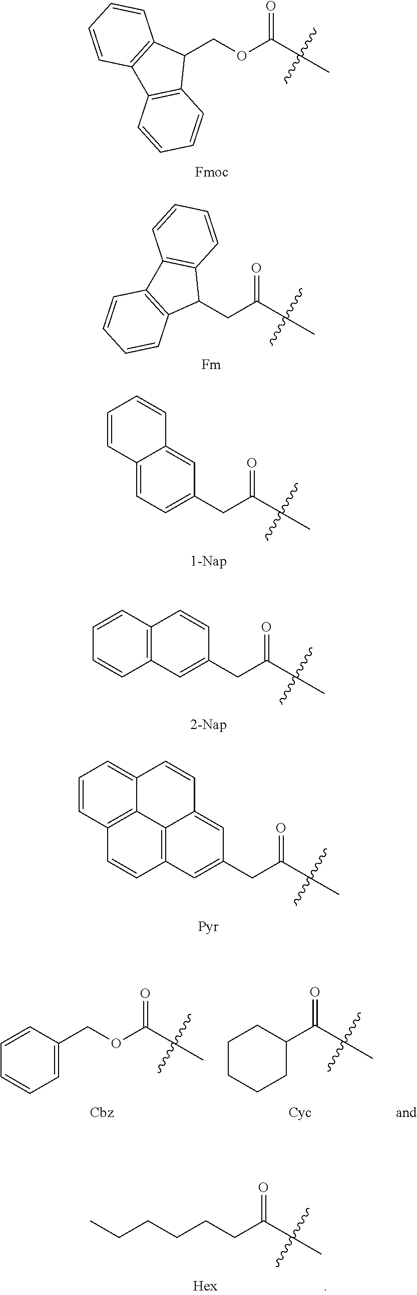

In another preferred embodiment, R₂ is selected from the group consisting of:

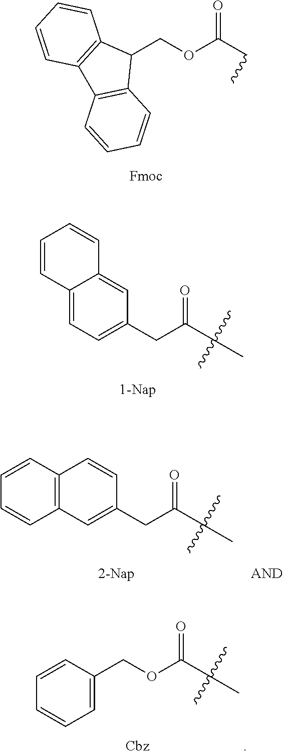

In yet another embodiment, R₁ is selected from the group consisting of:

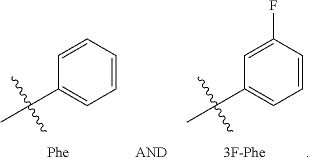 [SMILES: *c1ccccc1 ; *c1cccc(F)c1]

and R₂ is selected from the group consisting of:

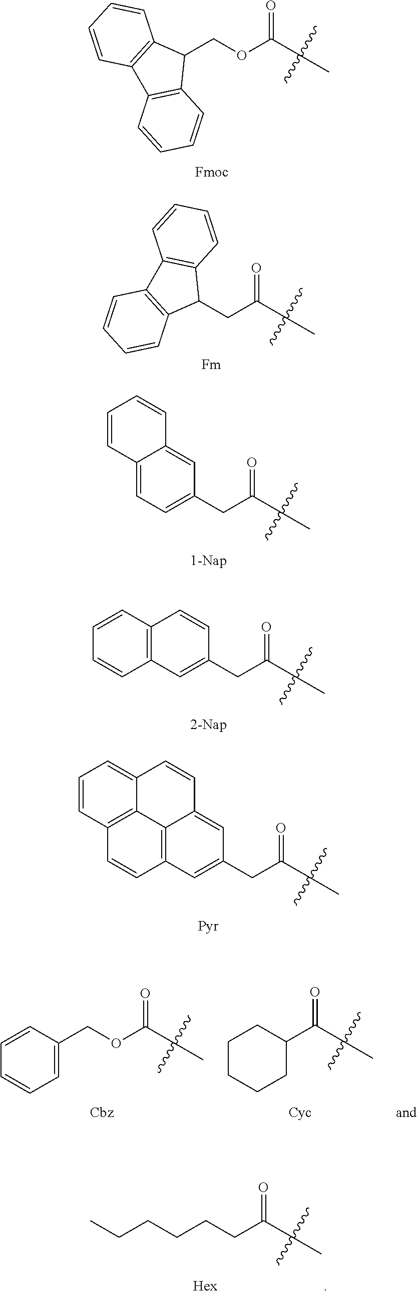

In yet another embodiment, R₁ is selected from the group consisting of:

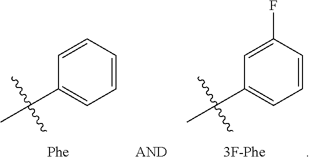 [SMILES: *c1ccccc1 ; *c1cccc(F)c1]

and R₂ is selected from the group consisting of:

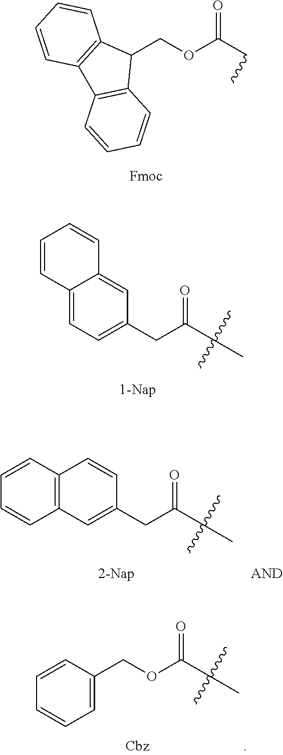

The disclosed nanoparticle complex may optionally contain more than one phenylalanine derivative.

In another embodiment, R₂ is

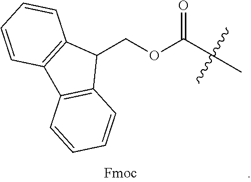 [SMILES: *C(=O)OCC1c2ccccc2-c2ccccc21]

In another embodiment, R₁ is

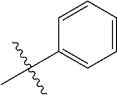 [SMILES: *c1ccccc1]

### Phe and R₂ is not

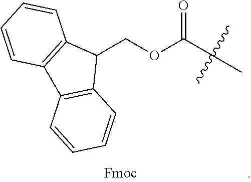 [SMILES: *C(=O)OCC1c2ccccc2-c2ccccc21]

In the disclosed nanoparticle complex, the diameter of the nanoparticles is preferably less than 500 nm, less than 250 nm, less than 150 nm or less than 100 nm.

Also disclosed in this application a method of delivering RNA or DNA, including chemically-modified derivatives of RNA or DNA, into a cell, the method comprising: (a) Mixing the RNA or DNA with one or more amino acid derivatives of Formula I, II, III, IV, V or VI above, wherein R₁ is a canonical or non-canonical amino acid; R₂ is a hydrophobic, aromatic or charged modifying group; R₃ is hydrophobic, aromatic or charged modifying group; and n is an integer from 1 to 4; (b) Diluting the mixture into a cell culture medium; and (c) Dosing the cell with the diluted mixture. In one embodiment of the method, R₂ is selected from the group consisting of:

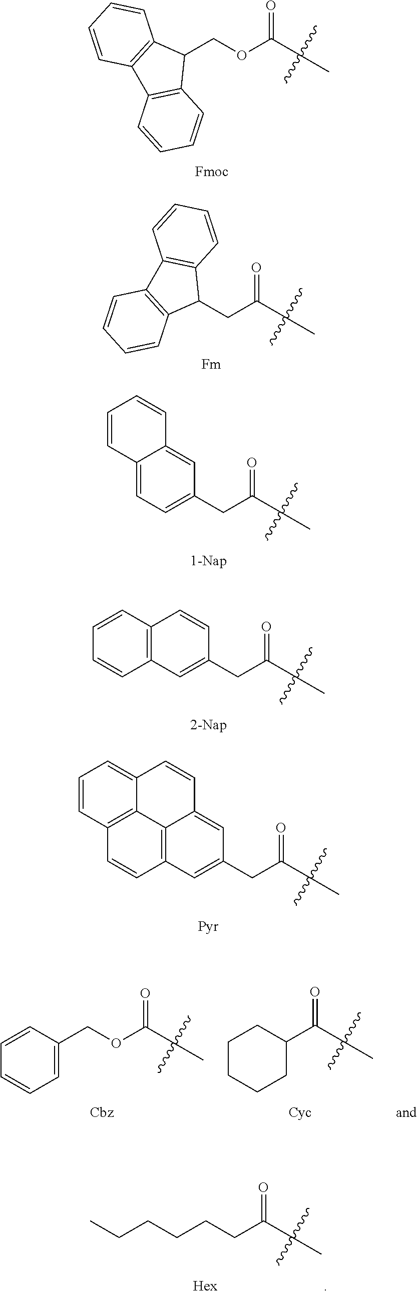

In another embodiment of the method, R₃ is selected from the group consisting of:

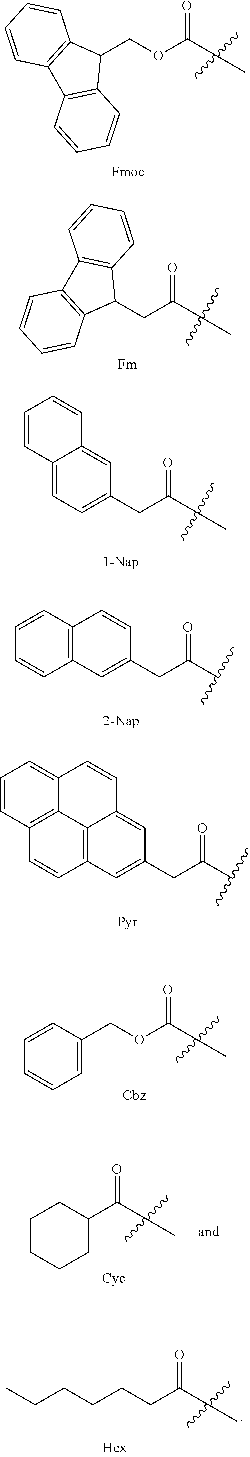

In another embodiment of the method, R₂ and R₃ are each selected from the group consisting of:

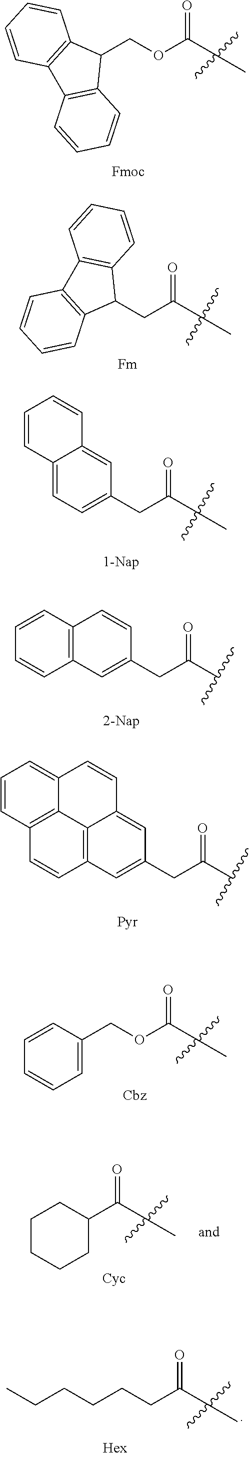

In one embodiment of the method, R₂ is selected from the group consisting of:

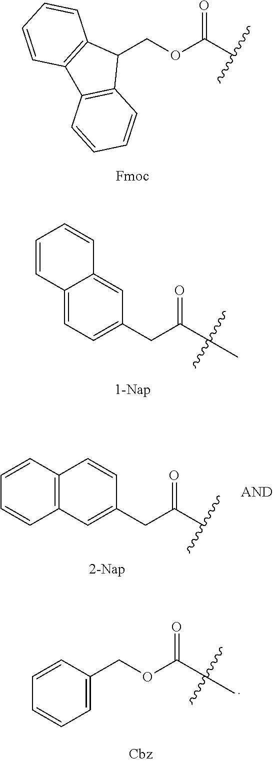 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In another embodiment of the method, R₃ is selected from the group consisting of:

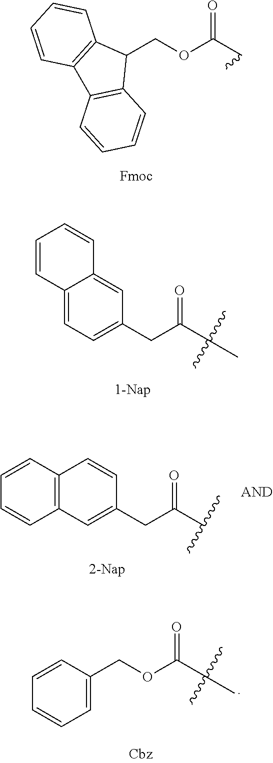

In another embodiment of the method, R₂ and R₃ are each selected from the group consisting of:

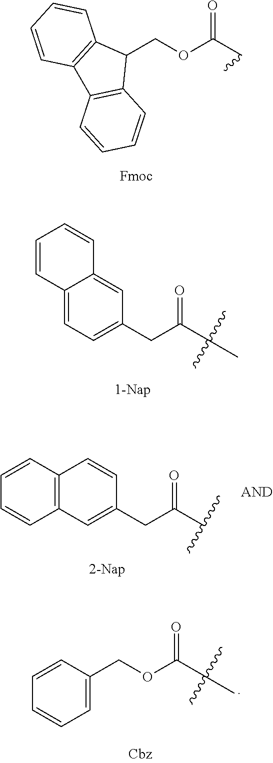

The disclosed method may use more than one amino acid derivative. In a preferred embodiment, the RNA is small interfering RNA (siRNA). Preferably, the method reduces messenger RNA (mRNA) levels and/or protein levels related to a specific gene or genes, preferably by at least 50%. Preferably, the method exhibits cell viabilities equal to or exceeding 40% after 48 hours, or more, preferably, equal to or exceeding 60% after 48 hours. The above method may be used to deliver various types of therapeutic RNA or DNA to the patient's cells, including mRNA, siRNA (and its variations), miRNA, short hairpin RNA (shRNA), and/or other oligonucleotides, including plasmid DNA and antisense oligonucleotides.

Also disclosed is a method of delivering RNA or DNA, including chemically-modified derivatives of RNA or DNA, into a cell, the method comprising: (a) Mixing the RNA or DNA with one or more phenylalanine derivatives with the formula:

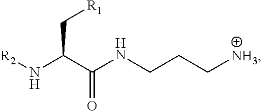

where R₁ is benzene or a fluorinated benzene; and R₂ is an N-terminal modifying group; (b) Diluting the mixture into a cell culture medium; and (c) Dosing the cell with the diluted mixture. In an embodiment of the method, R₁ is selected from the group consisting of:

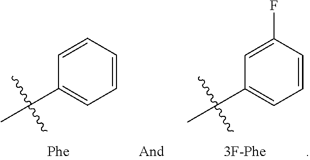 [SMILES: *c1ccccc1 ; *c1cccc(F)c1]

In another embodiment, R₂ is selected from the group consisting of:

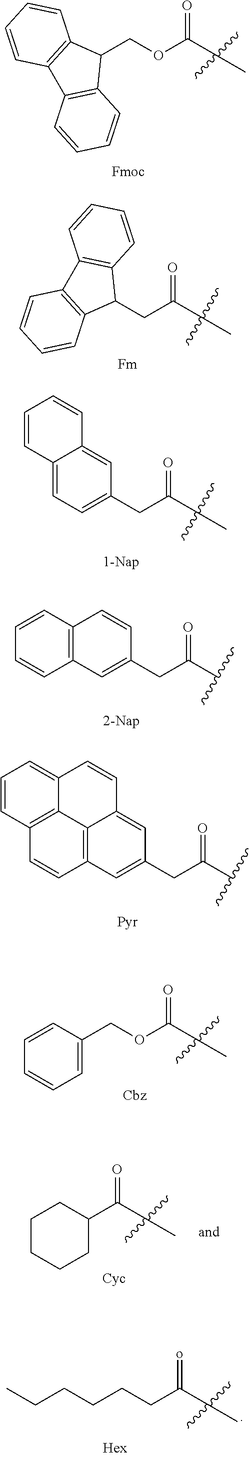

In another embodiment, R₂ is selected from the group consisting of:

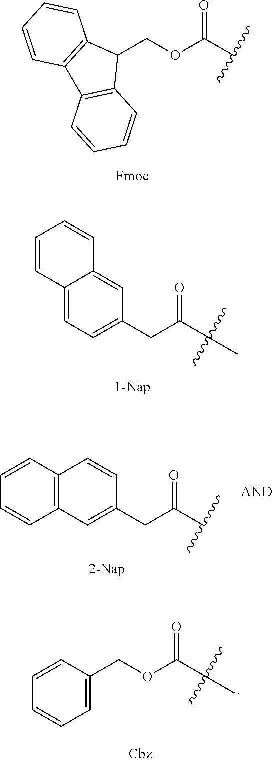 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In another embodiment, R₁ is selected from the group consisting of:

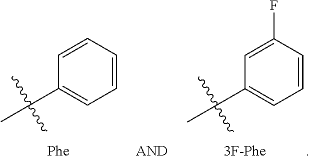 [SMILES: *c1ccccc1 ; *c1cccc(F)c1]

and R₂ is selected from the group consisting of:

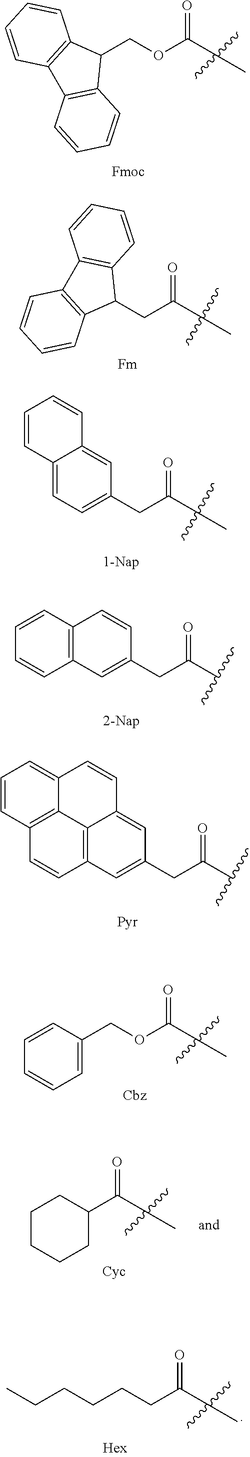

In another embodiment, R₁ is selected from the group consisting of:

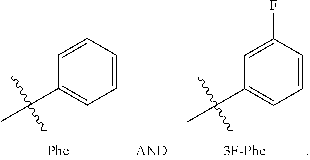 [SMILES: *c1ccccc1 ; *c1cccc(F)c1]

And
and R₂ is selected from the group consisting of:

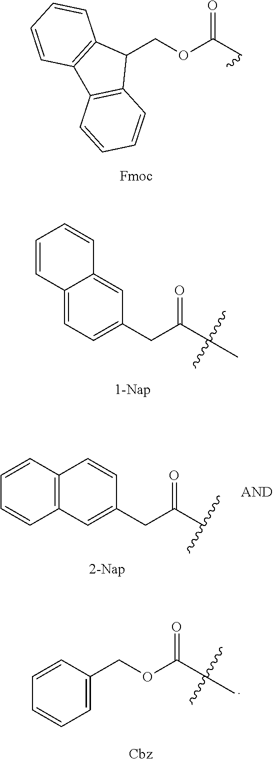

The disclosed complex may contain more than one phenylalanine derivative. In yet another embodiment, R₂ is

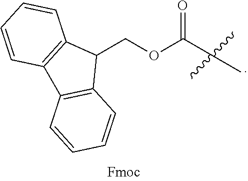 [SMILES: *C(=O)OCC1c2ccccc2-c2ccccc21]

In another embodiment, R₁ is

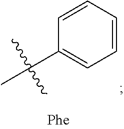 [SMILES: *c1ccccc1]

### and R₂ is not

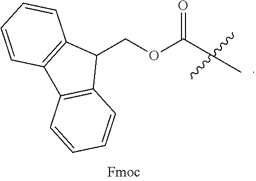 [SMILES: *C(=O)OCC1c2ccccc2-c2ccccc21]

In a preferred embodiment, the RNA is short interfering RNA (siRNA). Preferably, the method reduces messenger RNA (mRNA) levels and/or protein levels related to a specific gene or genes, more preferably by at least 50%. Preferably, the method exhibits cell viabilities equal to or exceeding 40% after 48 hours, or, more preferably, exceeding 60% after 48 hours. The above method may be used to deliver various types of therapeutic RNA or DNA to the patient's cells, including mRNA, siRNA (and its variations), miRNA, short hairpin RNA (shRNA), and/or other oligonucleotides, including plasmid DNA and antisense oligonucleotides.

Also disclosed is a method of treating disease caused by expression of excessive or dysfunctional proteins, comprising: (a) Mixing RNA or DNA, including chemically-modified derivatives of RNA or DNA, with one or more amino acid derivatives of Formula I, II, III, IV, V or VI, where R₁ is a canonical or non-canonical amino acid; R₂ is a hydrophobic, aromatic or charged modifying group; R₃ is hydrophobic, aromatic or charged modifying group; and n is an integer from 1 to 4; (b) Diluting the mixture into a medium; and (c) Delivering the diluted mixture to a tissue of a subject, thereby reducing harmful protein expression or increasing beneficial protein expression. In one embodiment, R₂ is selected from the group consisting of:

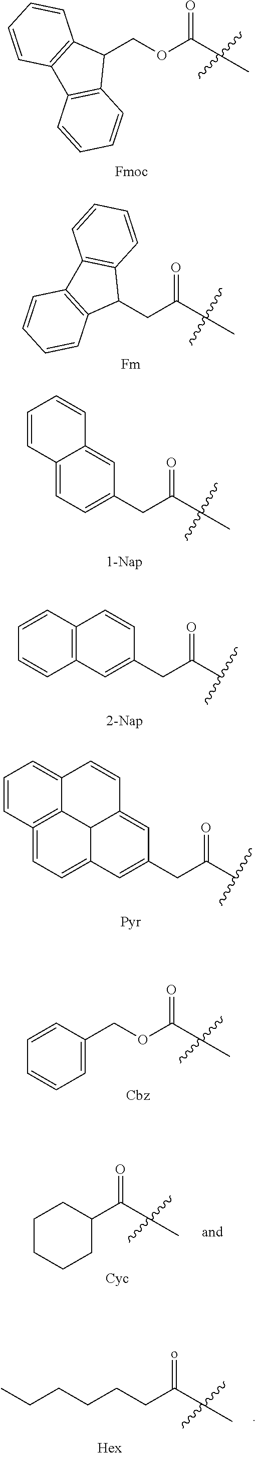

In another embodiment, R₃ is selected from the group consisting of:

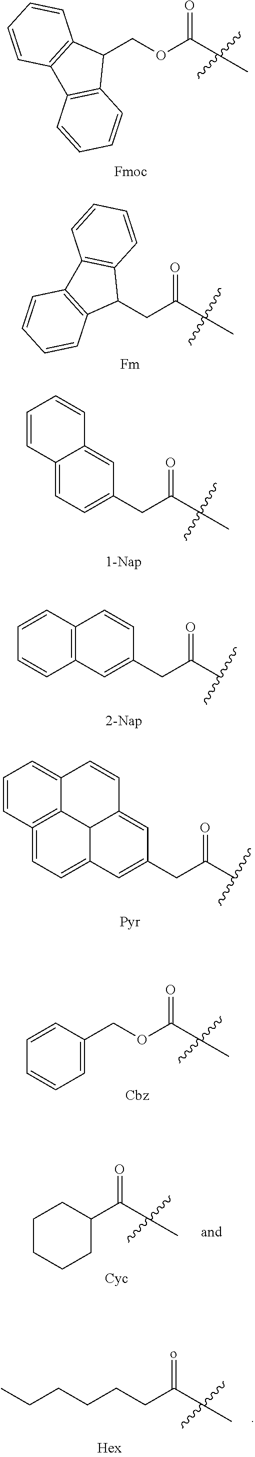

In yet another embodiment, R₂ and R₃ are each selected from the group consisting of:

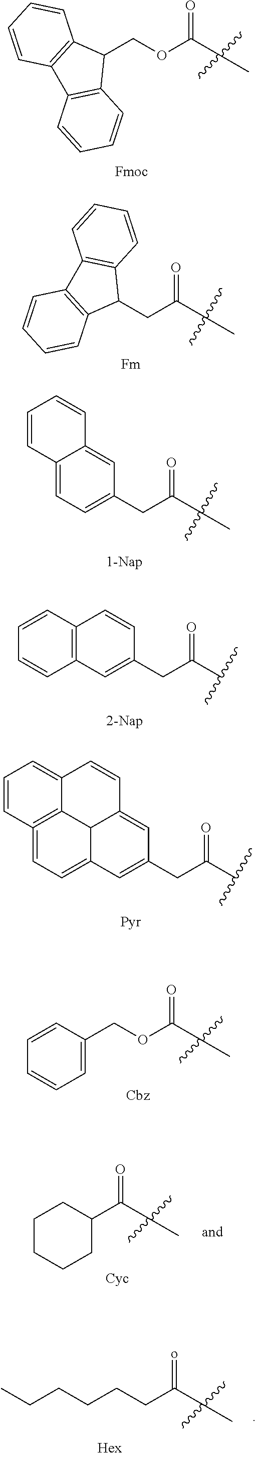

In one embodiment, R₂ is selected from the group consisting of:

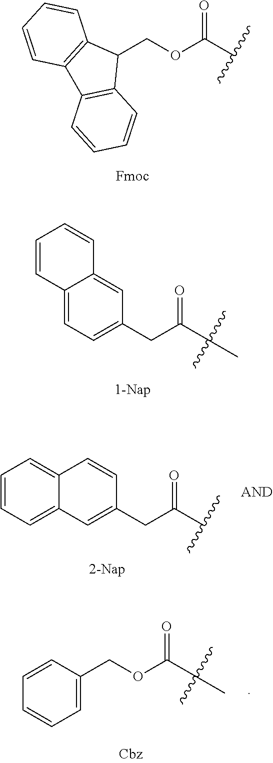 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In one embodiment R₂ is selected from the group consisting of:

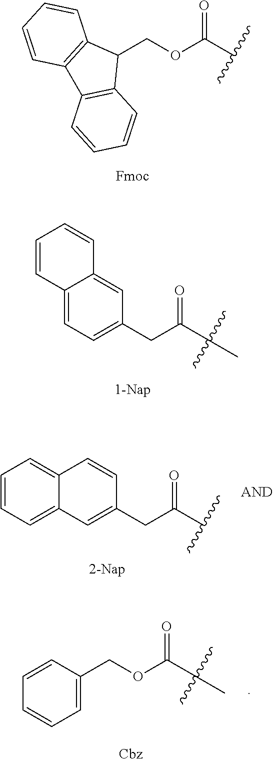 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In yet another embodiment, R₂ and R₃ are each selected from the group consisting of:

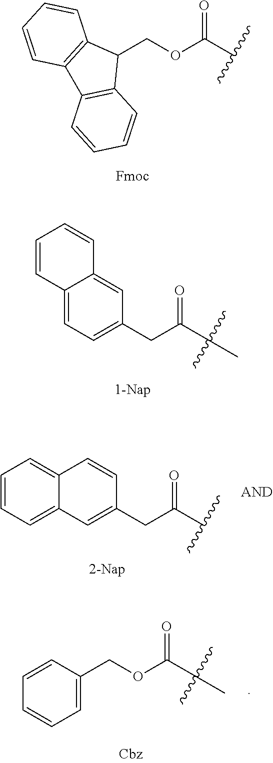 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

The disclosed method may use more than one amino acid derivative. In one embodiment of the method, the RNA is short interfering RNA (siRNA). Preferably, the method reduces messenger RNA (mRNA) levels and/or protein levels related to a specific gene or genes, more preferably by at least 50%. The subject of the treatment may be a mammal, preferably a human. The disease caused by expression of excessive or dysfunctional proteins may be cancer, Huntington's Disease, coronaviruses or other viruses, Alzheimer's disease, cystic fibrosis or Acute Respiratory Distress Syndrome (ARDS). In one embodiment, the disease treated is lung cancer. The above method may be used to deliver various types of therapeutic RNA or DNA to the patient's cells, including mRNA, siRNA (and its variations), miRNA, short hairpin RNA (shRNA), and/or other oligonucleotides, including plasmid DNA and antisense oligonucleotides.

Also disclosed is a method of treating disease caused by expression of excessive or dysfunctional proteins, comprising: (a) Mixing RNA or DNA, including chemically-modified derivatives of RNA or DNA, with one or more phenylalanine derivatives with the formula:

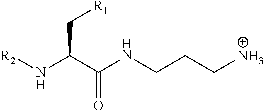

where R₁ is benzene or a fluorinated benzene; and R₂ is an N-terminal modifying group; (b) Diluting the mixture into a medium; and (c) Delivering the diluted mixture to a tissue of a subject, thereby reducing harmful protein expression or increasing beneficial protein expression. In one embodiment of the method, R₁ is selected from the group consisting of:

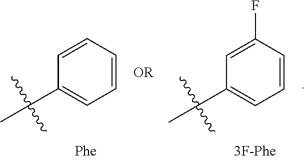 [SMILES: *c1ccccc1 ; *c1cccc(F)c1]

In another embodiment of the method R₂ is selected from the group consisting of:

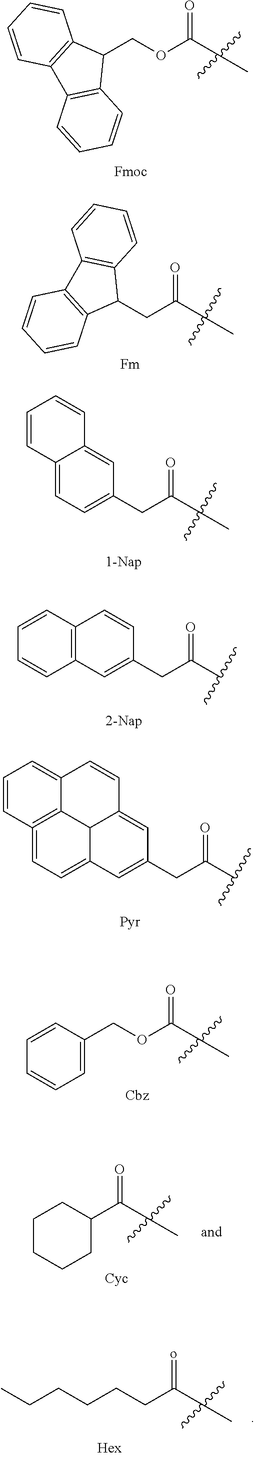

In another embodiment of the method R₂ is selected from the group consisting of:

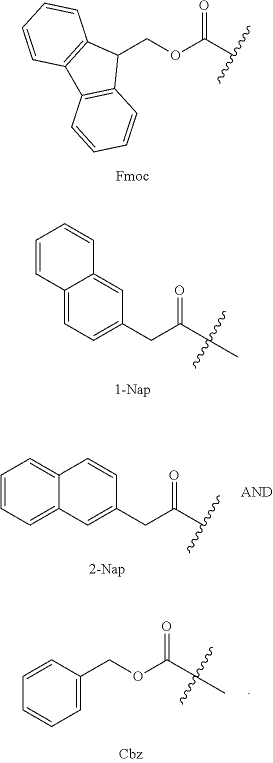 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In another embodiment of the method, R₁ is selected from the group consisting of:

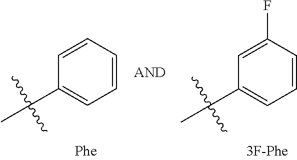 [SMILES: *c1ccccc1 ; *c1cccc(F)c1]

and R₂ is selected from the group consisting of:

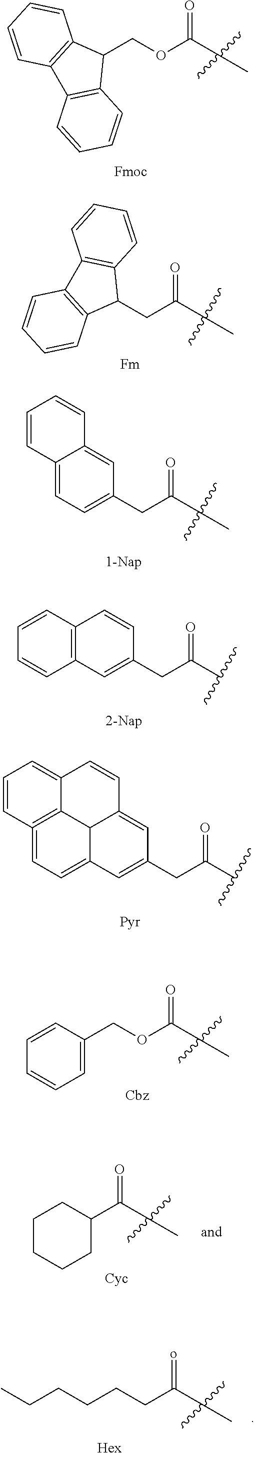

In another embodiment of the method, R₁ is selected from the group consisting of:

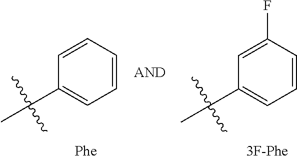 [SMILES: *c1ccccc1 ; *c1cccc(F)c1]

and R₂ is selected from the group consisting of:

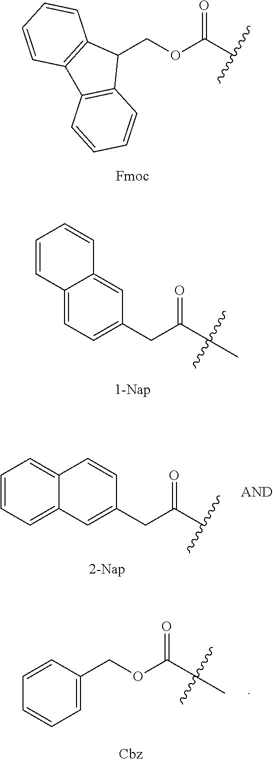 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

The disclosed complex may contain more than one phenylalanine derivative. In another embodiment of the method, R₂ may be

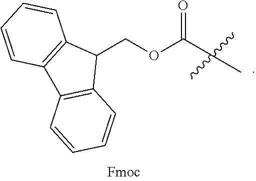 [SMILES: *C(=O)OCC1c2ccccc2-c2ccccc21]

In another embodiment of the invention, R₁ is

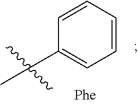 [SMILES: *c1ccccc1]

### and R₂ is not

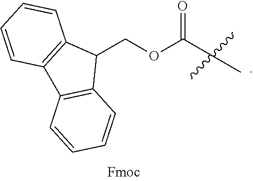 [SMILES: *C(=O)OCC1c2ccccc2-c2ccccc21]

In one embodiment of the method, the RNA is short interfering RNA (siRNA). The method may reduce messenger RNA (mRNA) levels and/or protein levels related to a specific gene or genes, preferably by at least 50%. The subject of the treatment may be a mammal, preferably a human. The disease caused by expression of excessive or dysfunctional proteins may be cancer, Huntington's Disease, coronaviruses or other viruses, Alzheimer's disease, cystic fibrosis or Acute Respiratory Distress Syndrome (ARDS). In one embodiment, the disease treated is lung cancer. The above method may be used to deliver various types of therapeutic RNA or DNA to the patient's cells, including mRNA, siRNA (and its variations), miRNA, short hairpin RNA (shRNA), and/or other oligonucleotides, including plasmid DNA and antisense oligonucleotides.

Another embodiment is a nanoparticle complex comprising: therapeutic RNA or DNA and one or more amino acid derivatives of Formula I, II, III, IV, V or VI, where R₁ is a canonical or non-canonical amino acid; R₂ is a hydrophobic, aromatic or charged modifying group; R₃ is hydrophobic, aromatic or charged modifying group; and n is an integer from 1 to 4.

In one embodiment, R₂ is selected from the group consisting of:

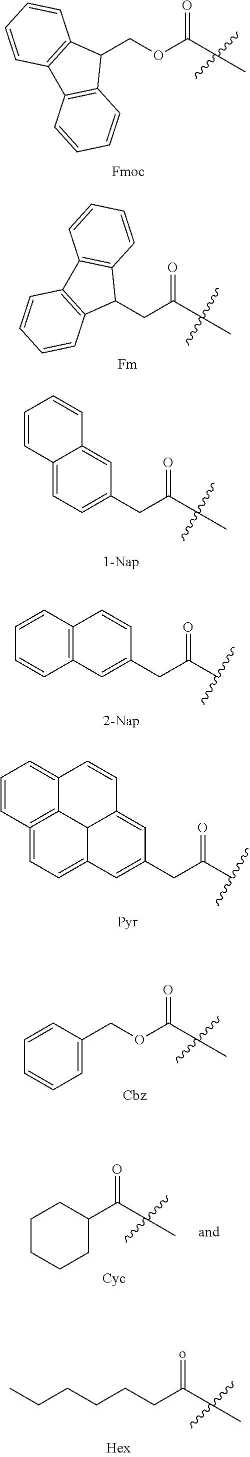

In another embodiment, R₂ is selected from the group consisting of:

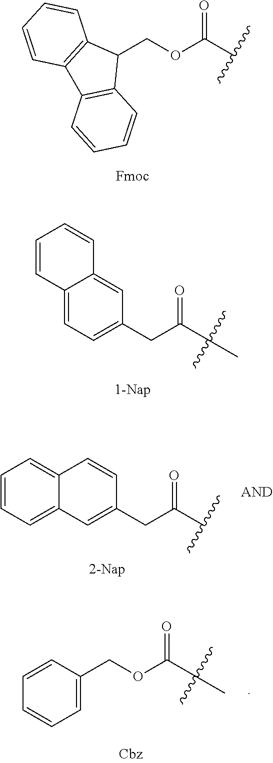 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In another embodiment, R₃ is selected from the group consisting of:

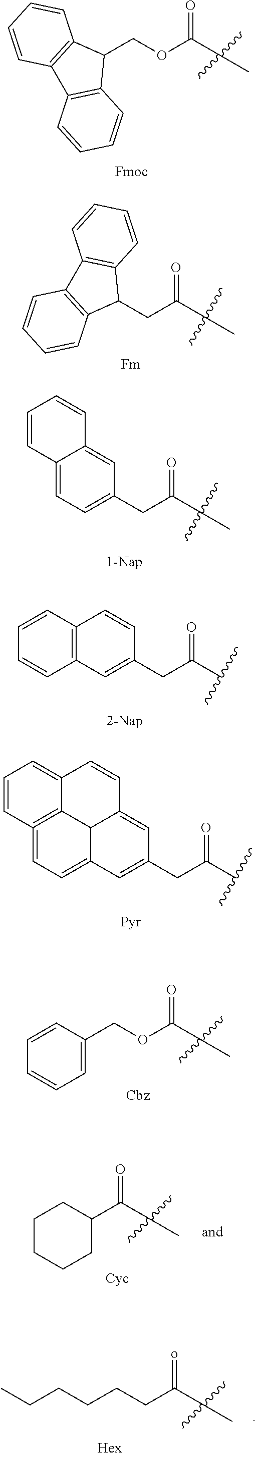

In another embodiment, R₃ is selected from the group consisting of:

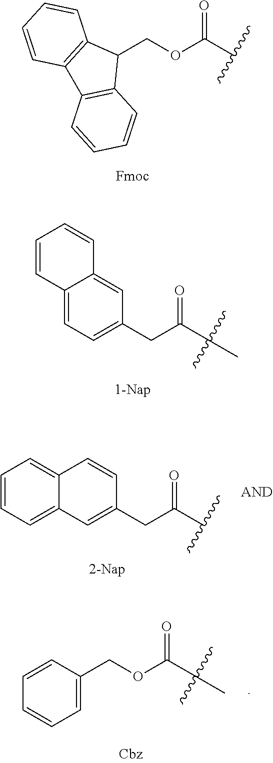 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In yet another embodiment, R₂ and R₃ are each selected from the group consisting of:

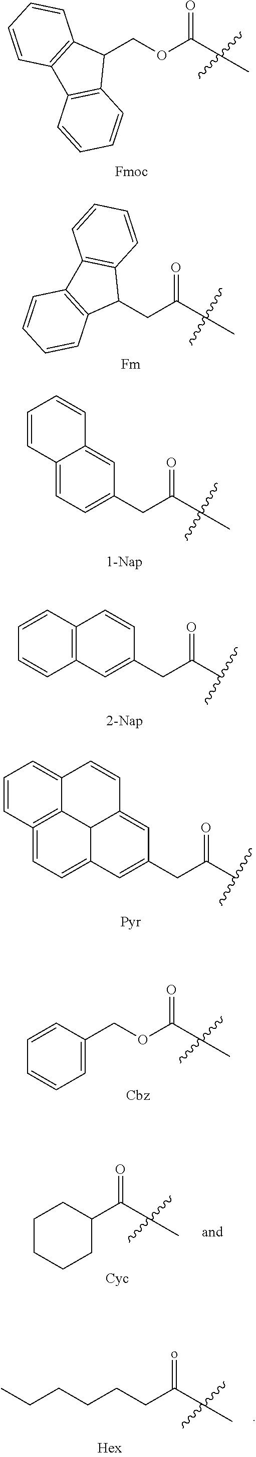

In yet another embodiment, R₂ and R₃ are each selected from the group consisting of:

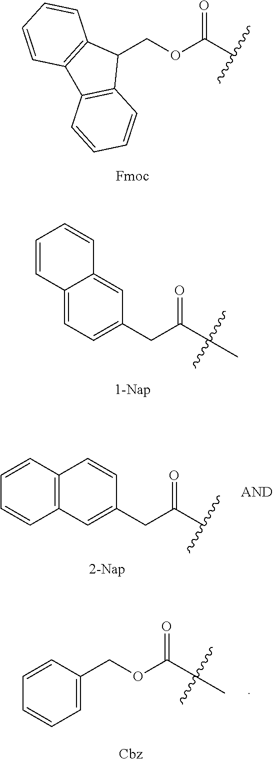 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

The disclosed complex may contain more than one amino acid derivative. In different embodiments, the disclosed nanoparticle complex may contain spherical or micelle-like nanoparticles, a mixture of spherical/micelle-like and fibril-like nanoparticles, or a mixture of spherical/micelle-like, sheet-like and/or fibril-like nanoparticles. In one embodiment of the nanoparticle complex, the RNA or DNA may comprise antisense oligonucleotides, messenger RNA (mRNA), short interfering RNA (siRNA) or plasmid DNA (pDNA).

Another embodiment disclosed is a nanoparticle complex comprising: therapeutic RNA or DNA and one or more phenylalanine derivatives with the formula:

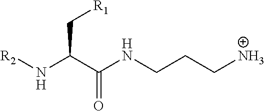

where R₁ is benzene or a fluorinated benzene; and R₂ is an N-terminal modifying group. In one embodiment, R₁ is selected from the group consisting of:

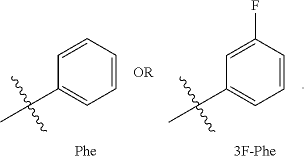 [SMILES: *c1ccccc1 ; *c1cccc(F)c1]

In another embodiment, R₂ is selected from the group consisting of:

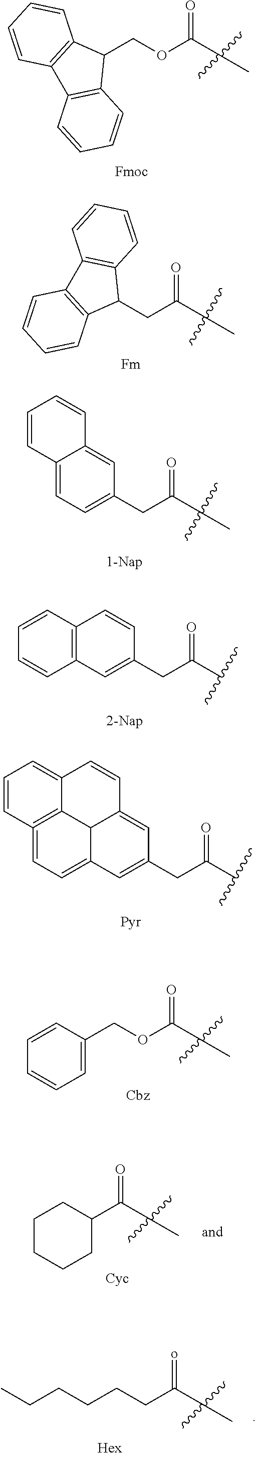

In another embodiment, R₂ is selected from the group consisting of:

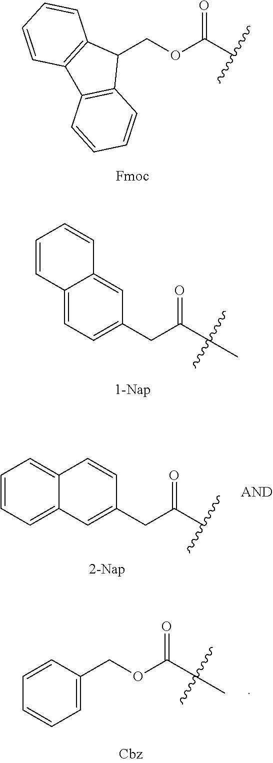 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

In yet another embodiment, R₁ is selected from the group consisting of:

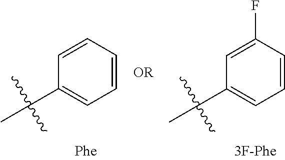 [SMILES: *c1ccccc1 ; *c1cccc(F)c1]

and R₂ is selected from the group consisting of:

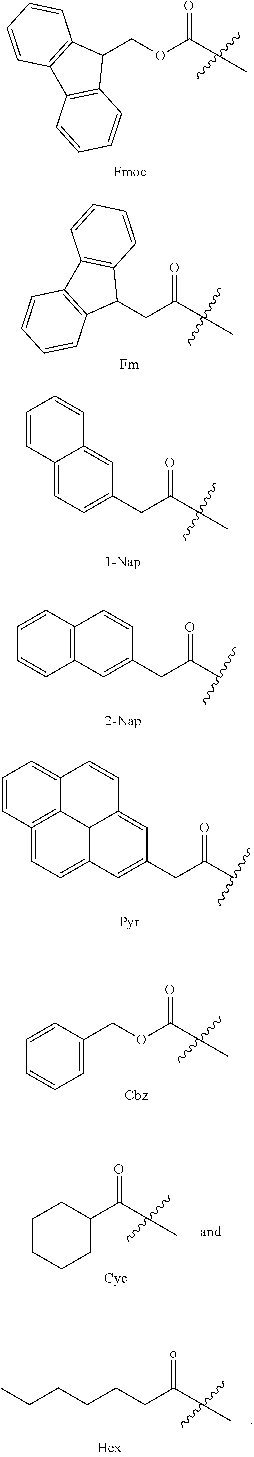

In yet another embodiment, R₁ is selected from the group consisting of:

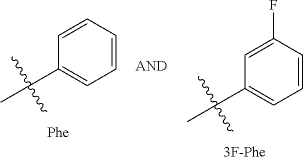 [SMILES: *c1ccccc1 ; *c1cccc(F)c1]

and R₂ is selected from the group consisting of:

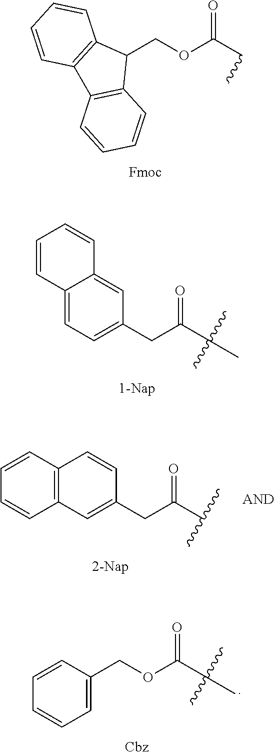

The nanoparticle complex may contain more than one phenylalanine derivative. In on embodiment, R₂ is

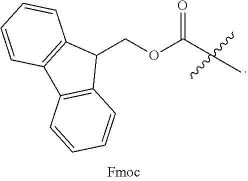 [SMILES: *C(=O)OCC1c2ccccc2-c2ccccc21]

In another embodiment, R₁ is

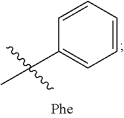 [SMILES: *c1ccccc1]

### and R₂ is not

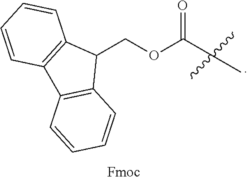 [SMILES: *C(=O)OCC1c2ccccc2-c2ccccc21]

In embodiments of the nanoparticle complex, the diameter of the nanoparticles may be less than 500 nm, less than 250 nm, less than 150 nm, or less than 100 nm. In embodiments of the nanoparticle complex, the RNA or DNA may comprise antisense oligonucleotides, messenger RNA (mRNA), siRNA or plasmid DNA (pDNA).

### IV. BRIEF DESCRIPTION OF THE DRAWINGS

FIG. 1 shows an overview of RNAi.

FIG. 2 shows major endocytic uptake mechanisms.

FIG. 3 shows the chemical structures of the disclosed N-X-Phe-DAP derivatives.

FIG. 4 Representative TEM of N-X-Phe-DAP derivatives.

FIG. 5 shows cellular uptake of FITC-labelled siRNA (FITC-siRNA, green) in A549 lung adenocarcinoma cells by N-X-Phe-DAP derivatives.

FIG. 6 shows knockdown efficacy of N-X-Phe-DAP/siRNA complexes against TTF-1 expression in A549 lung adenocarcinoma cells.

FIG. 7 shows cytotoxicity of Lipofectamine and N-X-Phe-DAP derivatives via MTT assay.

FIG. 8 shows cytotoxicity of Lipofectamine and N-X-Phe-DAP derivatives via MTT assay, reporting cell viability reported after dosing for 4 h, followed by media change with 1×DMEM, and additional incubation for 48 h.

FIG. 9 shows the general synthetic strategy for the preparation of low molecular weight phenylalanine derivatives.

FIG. 10 Structure of Fm-Phe-DAP.

FIG. 11 ¹H NMR of Fm-Phe-DAP.

FIG. 12 ¹³C NMR of Fm-Phe-DAP.

FIG. 13 High resolution mass spectrum of Fm-Phe-DAP.

FIG. 14 Structure of 1-Nap-Phe-DAP.

FIG. 15 ¹H NMR of 1-Nap-Phe-DAP.

FIG. 16 ¹³C NMR of 1-Nap-Phe-DAP.

FIG. 17 High resolution mass spectrum of 1-Nap-Phe-DAP.

FIG. 18 Structure of 2-Nap-Phe-DAP.

FIG. 19 ¹H NMR of 2-Nap-Phe-DAP.

FIG. 20 ¹³C NMR of 2-Nap-Phe-DAP.

FIG. 21 High resolution mass spectrum of 2-Nap-Phe-DAP.

FIG. 22 Structure of Pyr-Phe-DAP.

FIG. 23 ¹H NMR of Pyr-Phe-DAP.

FIG. 24 ¹³C NMR of Pyr-Phe-DAP.

FIG. 25 High resolution mass spectrum of Pyr-Phe-DAP.

FIG. 26 Structure of Cbz-Phe-DAP.

FIG. 27 ¹H NMR of Cbz-Phe-DAP.

FIG. 28 ¹³C NMR of Cbz-Phe-DAP.

FIG. 29 High resolution mass spectrum of Cbz-Phe-DAP.

FIG. 30 Structure of Cyc-Phe-DAP.

FIG. 31 ¹H NMR of Cyc-Phe-DAP.

FIG. 32 ¹³C NMR of Cyc-Phe-DAP.

FIG. 33 High resolution mass spectrum of Cyc-Phe-DAP.

FIG. 34 Structure of Hex-Phe-DAP.

FIG. 35 ¹H NMR of Hex-Phe-DAP.

FIG. 36 ¹³C NMR of Hex-Phe-DAP.

FIG. 37 High resolution mass spectrum of Hex-Phe-DAP.

FIG. 38 Complex size of N-X-Phe-DAP/siRNA nanoparticles.

FIG. 39 Nitrocellulose (left) and nylon (right) membranes for Fmoc-Phe-DAP.

FIG. 40 Nitrocellulose (left) and nylon (right) membranes for Fmoc-3F-Phe-DAP.

FIG. 41 Nitrocellulose (left) and nylon (right) membranes for Fmoc-F₅-Phe-DAP.

FIG. 42 Nitrocellulose (left) and nylon (right) membranes for Fm-Phe-DAP.

FIG. 43 Nitrocellulose (left) and nylon (right) membranes for 1-Nap-Phe-DAP.

FIG. 44 Nitrocellulose (left) and nylon (right) membranes for 2-Nap-Phe-DAP.

FIG. 45 Nitrocellulose (left) and nylon (right) membranes for Pyr-Phe-DAP.

FIG. 46 Nitrocellulose (left) and nylon (right) membranes for Cbz-Phe-DAP.

FIG. 47 Nitrocellulose (left) and nylon (right) membranes for Cyc-Phe-DAP.

FIG. 48 Nitrocellulose (left) and nylon (right) membranes for Hex-Phe-DAP.

FIG. 49 shows the general chemical structure of amino acid derivatives.

FIG. 50 are images of cells treated with a mixture of 1-Nap-Phe-DAP with fluorescent protein encoding mRNA (Panel A) and plasmid DNA (Panel B).

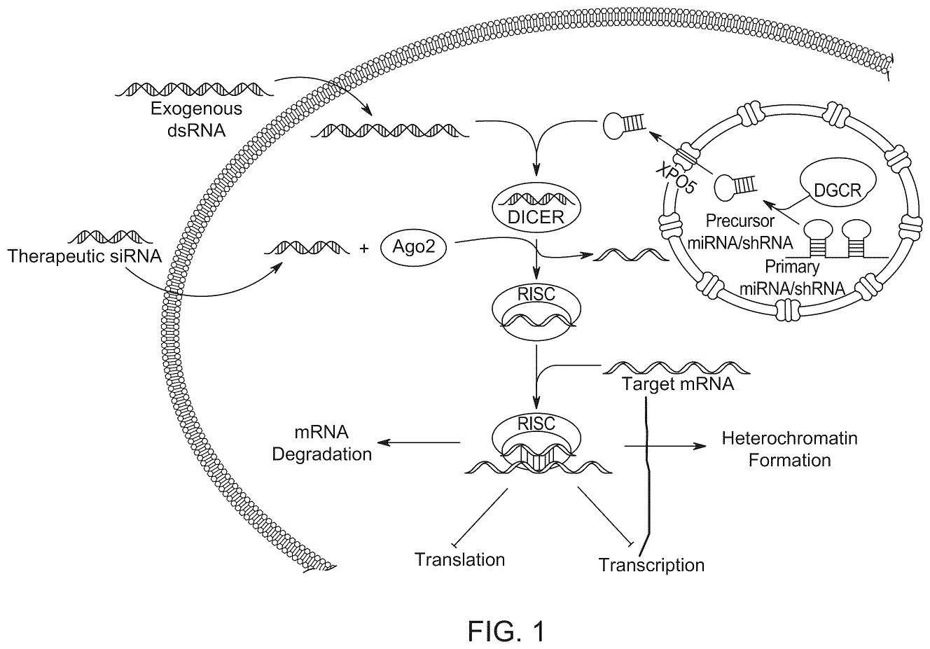

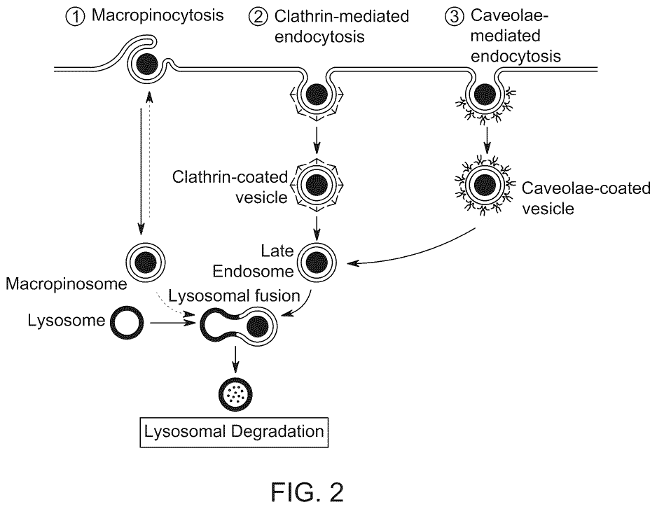

### V. DETAILED DESCRIPTION

Although significant progress in RNAi delivery has been made with several types of transfection agents, significant barriers have thus far limited clinical applications with these agents. Further, although a number cell-penetrating peptide (CPP) systems have been validated for in vitro and in vivo siRNA delivery, clinical applications remain limited by issues with limited serum half-life, cytotoxicity, and the high cost of peptide production. Inexpensive delivery agents that replicate the desirable properties of peptides for siRNA delivery would be of great value.

Disclosed are low molecular weight (<800 Da preferably <600 Da) phenylalanine (Phe) derivatives that condense with siRNA to form nanoparticles that facilitate cytosolic delivery and functional knockdown of target genes. These Phe derivatives have been modified at the N- and C-termini respectively with aromatic/hydrophobic and cationic functional groups that mirror common chemical properties of peptide-based delivery agents but at a fraction of the cost of peptide-based materials. These low molecular weight delivery agents may serve as low-cost alternatives to peptides for the functional delivery of siRNA for gene silencing applications.

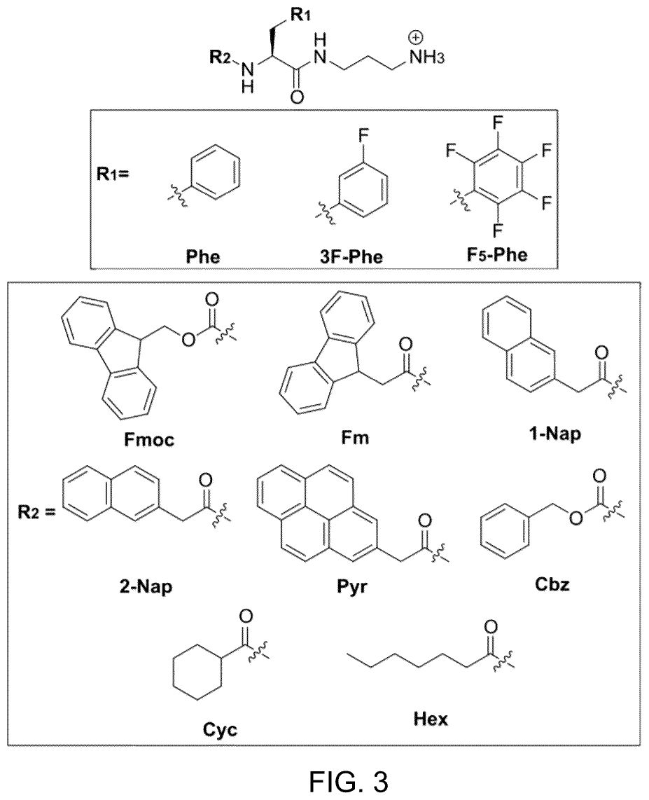

A targeted set of ten N-terminally modified phenylalanine derivatives with C-terminal diaminopropane (DAP) to impart cationic character (FIG. 3) were prepared, including Fmoc-Phe-DAP, Fmoc-3F-Phe-DAP, and Fmoc-F₅-Phe-DAP, which have previously been shown to self-assemble into supramolecular hydrogel networks. The remaining seven derivatives are composed of Phe-DAP with N-terminal modification that includes aromatic groups of varying surface area or spatial orientation (Fm-Phe-DAP, 1-Nap-Phe-DAP, 2-Nap-Phe-DAP, Pyr-Phe-DAP, or Cbz-Phe-DAP) or non-aromatic hydrophobic groups that are cyclic (Cyc-Phe-DAP) or acyclic (Hex-Phe-DAP). All derivatives are positively charged due to the common C-terminal ammonium cation. FIG. 3 shows the chemical structure of the ten derivatives.

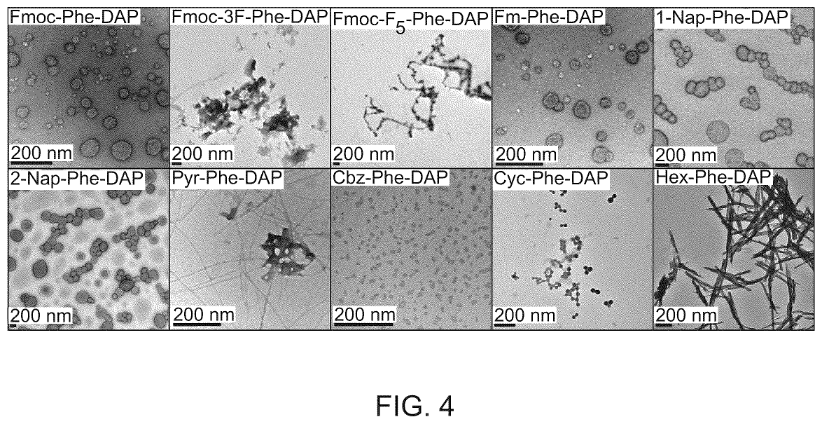

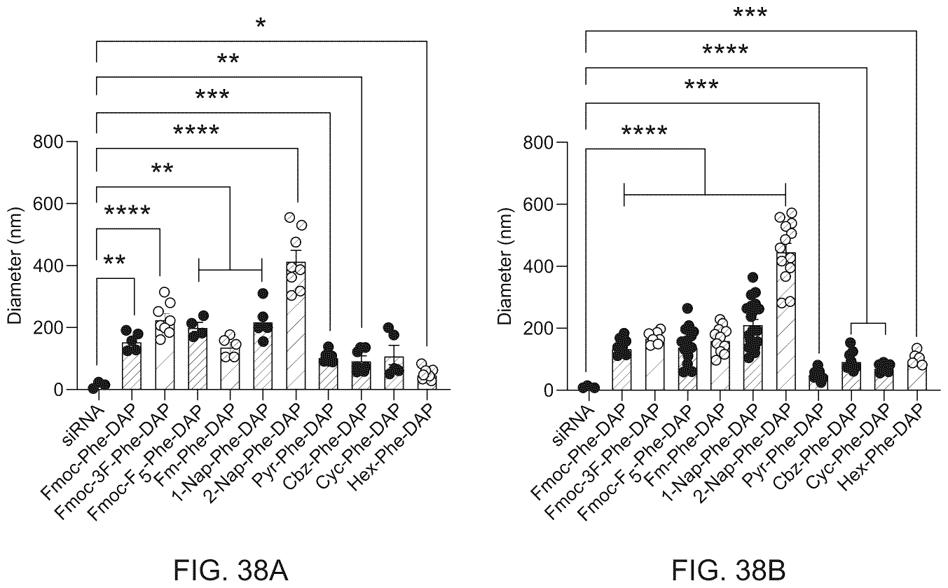

Each N-X-Phe-DAP derivative (100 μM) was mixed with siRNA (600 nM) in H₂O for 30 minutes, followed by dilution of this mixture into DMEM cell culture media to final concentrations of 5 μM N-X-Phe-DAP derivative and 30 nM siRNA. Dynamic light scattering (DLS) and transmission electron microscopy (TEM) were utilized to analyze the physical characteristics of the resulting N-X-Phe-DAP/siRNA nanoparticles (Table 2, FIG. 4). Table 2 shows the physical characteristics of N-X-Phe-DAP/siRNA nanoparticles. Average diameter (nm), determined by dynamic light scattering (DLS) and TEM imaging, is reported as the average of 20 measurements with the error reported as the standard deviation of the mean. Binding affinity (K_{d}) of each agent to siRNA was determined by a slot blot filtration assay with values reported as the average of three measurements and error reported as the standard deviation of the mean. FIG. 38 shows the complex size of N-X-Phe-DAP/siRNA nanoparticles: (A) Diameter (nm) determined by DLS (n=3, *p≤0.05, **p≤0.01, ***p≤0.001, ****p≤0.0001). (B) Diameter (nm) determined by TEM (n=9, ***p≤0.001 ****p≤0.0001).

TABLE 2

| | Diameter | Diameter | Binding Affinity |
|---|---|---|---|
| | (nm), DLS | (nm), TEM | (K_{d}), μM |
| Naked siRNA | 13 ± 6 | 10 ± 1 | — |
| Fmoc-Phe-DAP | 156 ± 13 | 136 ± 7 | 159 ± 11 |
| Fmoc-3F-Phe-DAP | 228 ± 18 | 170 ± 8 | 131 ± 41 |
| Fmoc-F₅-Phe-DAP | 202 ± 15 | 140 ± 12 | 217 ± 25 |
| Fm-Phe-DAP | 139 ± 14 | 163 ± 10 | 307 ± 34 |
| 1-Nap-Phe-DAP | 221 ± 26 | 211 ± 17 | 293 ± 79 |
| 2-Nap-Phe-DAP | 416 ± 33 | 450 ± 27 | 186 ± 45 |
| Pyr-Phe-DAP | 105 ± 8 | 50 ± 5.0 | 32 ± 1 |
| Cbz-Phe-DAP | 94 ± 14 | 93 ± 9 | 97 ± 3 |
| Cyc-Phe-DAP | 125 ± 74 | 71 ± 2 | 179 ± 8 |
| Hex-Phe-DAP | 50 ± 6 | 81 ± 10 | 183 ± 9 |

All derivatives formed complexes with siRNA and the properties of these nanoparticle complexes were dependent on the chemical structure of the N-X-Phe-DAP derivatives. Nanoparticles formed by complexation of siRNA with Fmoc-Phe-DAP, Fm-Phe-DAP, 1-Nap-Phe-DAP, and 2-Nap-Phe-DAP were spherical micelle-like structures that ranged from ˜150-450 nm in diameter by DLS and TEM measurements (Table 2). The siRNA nanoparticles of Cyc-Phe-DAP and Cbz-Phe-DAP were smaller spheres ˜100 nm in diameter (Table 2 and FIG. 4), with the Cyc-Phe-DAP/siRNA particles appearing to be more uniform and well-defined than the Cbz-Phe-DAP/siRNA particles. Interestingly, the aggregates formed with siRNA and Fmoc-3F-Phe-DAP, Fmoc-F₅-Phe-DAP, Pyr-Phe-DAP, and Hex-Phe-DAP showed mixtures of spherical particles and one-dimensional fibril-like structures (FIG. 4). Fmoc-Phe-DAP, Fmoc-3F-Phe-DAP, Fmoc-F₅-Phe-DAP have been previously shown to assemble into one-dimensional fibrils. Fmoc-Phe-DAP assembly into these types of fibrils was not observed in the presence of siRNA, while the assembly properties of Fmoc-3F-Phe-DAP and Fmoc-F₅-Phe-DAP were less affected by siRNA. Pyr-Phe-DAP-siRNA mixtures were dominated by fibril-like structures with some evidence of spherical nanoparticles (FIG. 4). Spherical nanoparticles are essentially absent from Hex-Phe-DAP-siRNA samples with images primarily exhibiting fibril and sheet-like structures that aggregate into bundles (FIG. 4).

The binding affinity (K_{d}) of N-X-Phe-DAP derivatives to siRNA was next characterized to determine how the chemical functionality of the Phe derivatives impacts siRNA interactions. Comparative binding affinity (K_{d}) of N-X-Phe-DAP derivatives to fluorescently labeled FITC-siRNA was determined using a slot blot filtration assay (Table 2). Monofluorination of the phenyl side chain (Fmoc-3F-Phe-DAP) had negligible impact on binding affinity compared to the non-fluorinated molecule (Fmoc-Phe-DAP) (131 μM and 159 μM, respectively). Benzyl perfluorination (Fmoc-F₅-Phe-DAP) drastically reduced binding to siRNA (217 μM). It has been previously demonstrated that Fmoc-F₅-Phe-DAP has a dramatically increased propensity to self-assemble into one-dimensional fibrils compared to Fmoc-Phe-DAP and Fmoc-3F-Phe-DAP. Therefore, the decrease in binding affinity of Fmoc-F₅-Phe-DAP for siRNA is likely due to competition between siRNA binding and self-assembly. Thus, decreasing electron density of the aromatic Phe side chain through fluorination did not improve siRNA binding properties of these derivatives.

Variation of the N-terminal modifying group also had an impact on the binding affinity of N-Phe-DAP derivatives to siRNA. Replacement of the Fmoc carbamate moiety of Fmoc-Phe-DAP with the amide-linked Fm fluorenyl group (Fm-Phe-DAP) that also has a linker group that is one oxygen atom shorter than Fmoc increases the K_{d} to >300 μM compared to 159 μM for Fmoc-Phe-DAP. Reduction of N-terminal aromatic surface area to a single benzyl ring with the benzyl carbamate group (Cbz-Phe-DAP) significantly improved siRNA binding (97 μM). The use of naphthalene amides as N-terminal modifying groups via attachment of a naphthalene modification at the 1-position (1-Nap-Phe-DAP) or 2-position (2-Nap-DAP) significantly reduced siRNA binding affinity (293 μM and 186 μM, respectively). The pyrene modified derivative, Pyr-Phe-DAP, has the largest aromatic surface area and exhibited the strongest siRNA binding affinity of all derivatives tested (32 μM). The strong binding affinity of Pyr-Phe-DAP for siRNA is likely due to the known ability of pyrene to intercalate with double-stranded oligonucleotides. Non-aromatic N-terminal derivatives (Cyc-Phe-DAP and Hex-Phe-DAP) had K_{d} values of ˜180 μM for siRNA by slot blot analysis. Thus, both aromatic and non-aromatic N-X-Phe-DAP derivatives were competent to interact with siRNA with aromatic character, hydrophobicity, and chemical spatial orientation all having an impact on binding affinity.

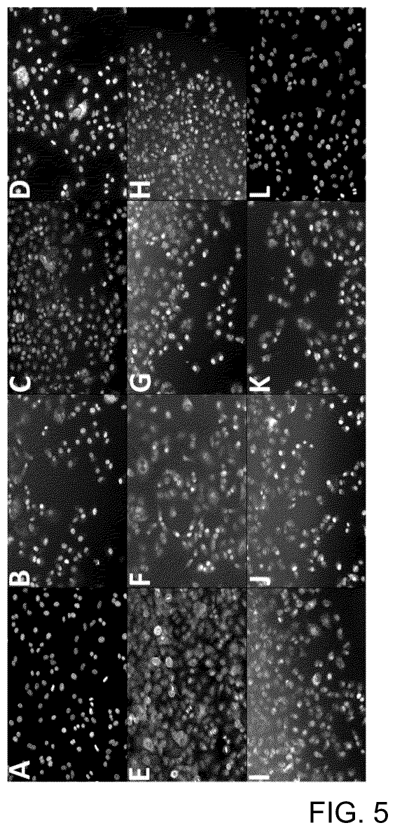

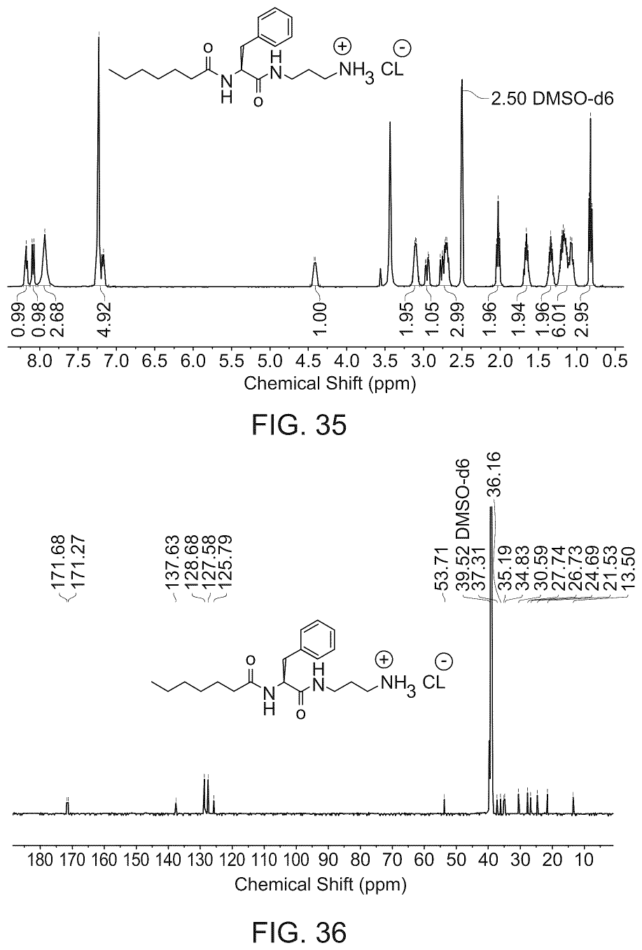

The N-X-Phe-DAP derivatives facilitate functional delivery of siRNA. N-X-Phe-DAP derivatives (100 μM) were incubated with a fluorescently labelled siRNA (FITC-siRNA, 600 nM) for 30 minutes in the dark followed by dilution into DMEM. A549 lung carcinoma cells were then dosed with these solutions for 4 hours, after which N-X-Phe-DAP/siRNA cellular uptake was visualized by confocal microscopy to visual FITC-siRNA in fixed cells in which the nuclei had been stained with DAPI nuclear stain (FIG. 5). Translocation of siRNA with the N-X-Phe-DAP derivatives was compared with Lipofectamine by observing the presence of green fluorescence (FITC-siRNA) in the images. The results demonstrate improved siRNA delivery with Fmoc-Phe-DAP (FIG. 5C), Fmoc-F₅-Phe-DAP (FIG. 5E), 1-Nap-Phe-DAP (FIG. 5G), Pyr-Phe-DAP (FIG. 5I), Cbz-Phe-DAP (FIG. 5J), and Hex-Phe-DAP (FIG. 5L). Delivery of FITC-siRNA with Fmoc-3F-Phe-DAP (FIG. 5D), Fm-Phe-DAP (FIG. 5F), 2-Nap-Phe-DAP (FIG. 5H), and Cyc-Phe-DAP (FIG. 5C) was on par with Lipofectamine 2-Nap-Phe-DAP (FIG. 35B). Overall, these results do not strongly correlate with complex size, morphology, and binding affinity. Additionally, there does not appear to be clear correlation between N-terminal modification and siRNA internalization.

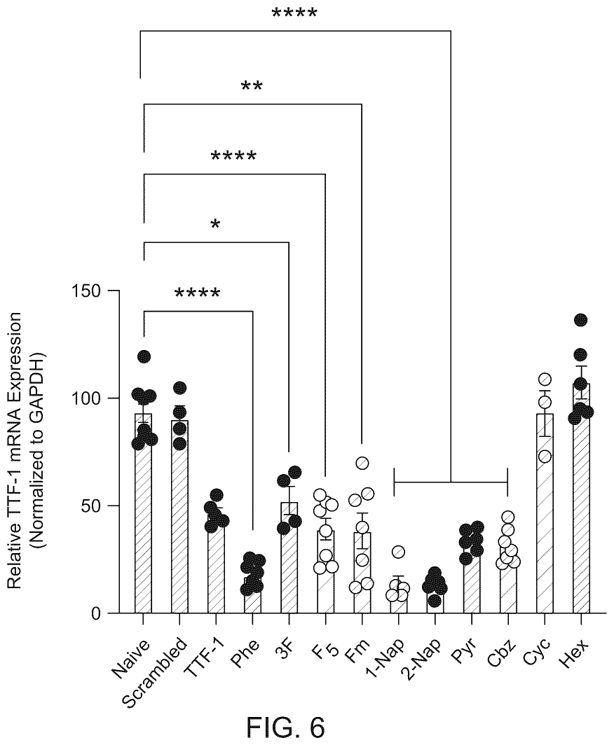

Finally, N-X-Phe derivatives achieved functional delivery of siRNA. To verify delivery of functional siRNA using N-X-Phe-DAP/siRNA particles, relative thyroid transcription factor-1 (TTF-1) mRNA expression normalized to GAPDH in A549 lung carcinoma cells was quantified against knockdown efficiencies with Lipofectamine as a control delivery agent (FIG. 6, Table 3). FIG. 6 shows Relative TTF-1 mRNA expression as percent knockdown normalized against GAPDH expression. “Scrambled” is control siRNA delivered with Lipofectamine. TTF-1 is delivery of TTF-1 siRNA with Lipofectamine (n=5, *p≤0.05, **p≤0.01, ****p≤0.0001).

Delivery of scrambled siRNA with Lipofectamine resulted in no significant reduction in TTF-1 mRNA levels, while delivery of TTF-1-specific siRNA with Lipofectamine reduced cellular TTF-1 mRNA by approximately 54%. The non-aromatic Phe derivatives, Cyc-Phe-DAP and Hex-Phe-DAP, failed to generate significant knockdown, showing <10% reduction of TTF-1 mRNA. Delivery of TTF-1 siRNA with Fmoc-3F-Phe-DAP produced results similar to Lipofectamine (˜48%), while Fmoc-F₅-Phe-DAP and Fm-Phe-DAP resulted in slight improvements in TTF-1 mRNA knockdown efficiency (˜61% and 62%, respectively). Additionally, TTF-1 expression was reduced by approximately 70% with Pyr-Phe-DAP and Cbz-Phe-DAP. Most significantly, Fmoc-Phe-DAP, 1-Nap-Phe-DAP, and 2-Nap derivatives resulted in 83%, 86% and 87% knockdown of TTF-1 mRNA, respectively.

Table 3 shows the knockdown efficacy of N-X-Phe-DAP derivatives in vitro.

TABLE 3

| | Relative TTF-1 mRNA Expression |
|---|---|
| | (Normalized to GAPDH) |
| Naïve | 93.0 ± 5.0 |
| Scrambled | 90.0 ± 5.6 |
| TTF-1 | 46.0 ± 2.4 |
| Fmoc-Phe-DAP | 17.4 ± 2.1 |
| Fmoc-3F-Phe-DAP | 51.8 ± 6.6 |
| Fmoc-F₅-Phe-DAP | 38.6 ± 4.9 |
| Fm-Phe-DAP | 37.9 ± 8.3 |
| 1-Nap-Phe-DAP | 13.8 ± 3.7 |
| 2-Nap-Phe-DAP | 13.4 ± 1.4 |
| Pyr-Phe-DAP | 33.0 ± 2.2 |
| Cbz-Phe-DAP | 30.9 ± 3.0 |
| Cyc-Phe-DAP | 92.3 ± 10.7 |
| Hex-Phe-DAP | 106.5 ± 7.4 |

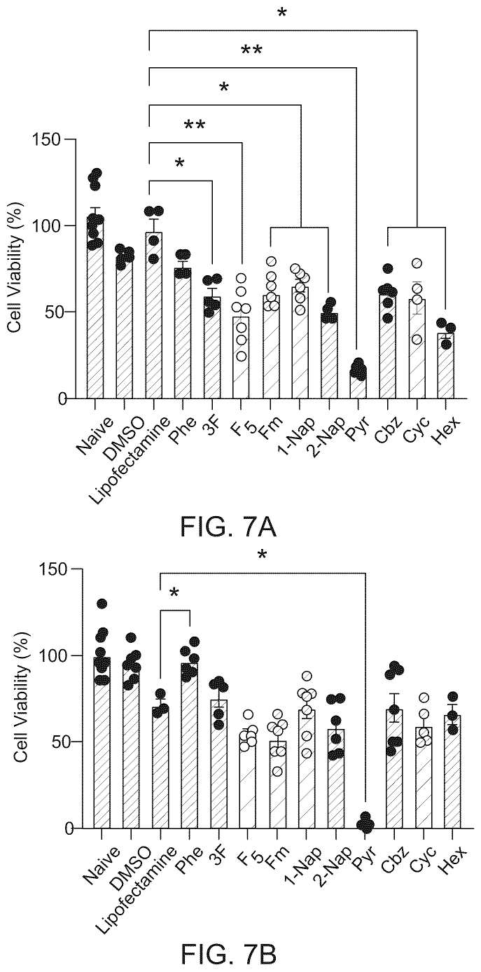

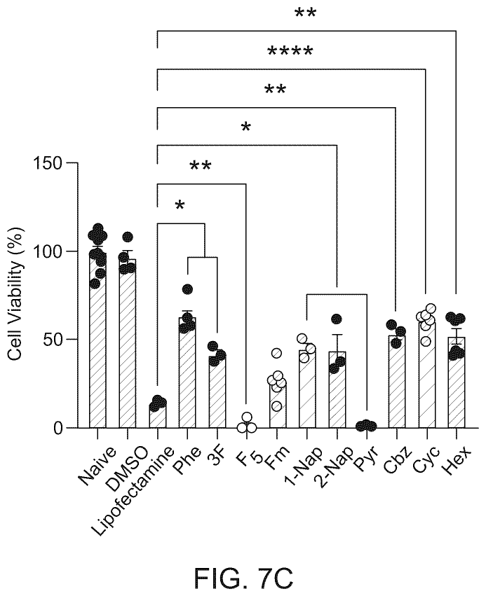

After demonstrating effective gene silencing, an MTT cytotoxicity assay was employed to evaluate cell viability in the presence of N-X-Phe-DAP derivatives. A549 cells were dosed with 100 M of these derivatives for 4 h (FIG. 7A), 24 h (FIG. 7B), and 48 h (FIG. 7C). FIG. 7 reports cell viability as percent viability of A549 cells normalized to naïve control. (A) Cell viability after 4 h dosing (n=3, *p≤0.05, **p≤0.01). (B) Cell viability after 24 h dosing (n=3, *p≤0.05, **p≤0.01). (C) Cell viability after 48 h dosing (n=3, *p≤0.05, **p≤0.01, ****p≤0.0001). Table 4 shows cell viability as determined for A549 cells following incubation with vehicle (DMSO), Lipofectamine, or N-X-Phe-DAP derivatives. An MTT assay was performed after dosing for 4 h, 24 h, 48 h, or 4 h with media change and additional 48 h incubation. Average values are reported with calculated standard error of the mean (SEM).

TABLE 4
Cytotoxicity of N-X-Phe-DAP derivatives via MTT Assay.

| | Cell Viability (%) | | | |
|---|---|---|---|---|
| | | | | Media Change After |
| | | | | 4 h Followed by |
| | 4 h Dosing | 24 h Dosing | 48 h Dosing | 48 h Incubation |
| Naïve | 106.0 ± 4.4 | 100.0 ± 3.6 | 100.0 ± 3.5 | 101.2 ± 2.2 |
| DMSO | 82.7 ± 1.3 | 94.9 ± 3.4 | 96.4 ± 4.2 | 78.9 ± 7.1 |
| Lipofectamine | 97.1 ± 6.8 | 71.2 ± 3.5 | 14.0 ± 1.1 | 36.9 ± 2.8 |
| Fmoc-Phe-DAP | 76.3 ± 2.8 | 96.5 ± 3.2 | 63.7 ± 5.1 | 76.1 ± 4.9 |
| Fmoc-3F-Phe-DAP | 59.9 ± 3.8 | 75.3 ± 5.0 | 42.0 ± 2.4 | 71.1 ± 6.3 |
| Fmoc-F₅-Phe-DAP | 48.1 ± 5.9 | 54.8 ± 3.2 | 1.3 ± 1.0 | 54.2 ± 6.2 |
| Fm-Phe-DAP | 60.7 ± 3.6 | 51.7 ± 4.4 | 26.7 ± 4.0 | 72.6 ± 7.4 |
| 1-Nap-Phe-DAP | 65.3 ± 3.7 | 69.5 ± 5.9 | 45.1 ± 3.1 | 56.5 ± 1.1 |
| 2-Nap-Phe-DAP | 50.4 ± 1.5 | 58.3 ± 6.0 | 44.3 ± 8.7 | 60.7 ± 1.6 |
| Pyr-Phe-DAP | 17.2 ± 1.1 | 2.6 ± 0.8 | 1.2 ± 0.1 | 22.3 ± 2.7 |
| Cbz-Phe-DAP | 60.8 ± 3.8 | 69.8 ± 8.2 | 53.7 ± 3.0 | 50.2 ± 3.4 |
| Cyc-Phe-DAP | 58.3 ± 9.2 | 59.6 ± 4.9 | 60.4 ± 2.6 | 66.5 ± 5.1 |
| Hex-Phe-DAP | 38.9 ± 3.7 | 66.0 ± 5.5 | 52.1 ± 4.5 | 53.1 ± 4.5 |

After 48 hours, Lipofectamine-treated cells exhibited only 14% viability, indicating the strong cytotoxicity of this commonly used transfection agent. Eight of the ten N-X-Phe-DAP derivatives were significantly less cytotoxic than the Lipofectamine control. Only Fmoc-F₅-Phe-DAP and Pyr-Phe-DAP were the more cytotoxic than Lipofectamine, with less than 2% of cells surviving 48 h of dosing. Although cells exposed to Fmoc-F₅-Phe-DAP retained over 40% viability after 24 h, less than 20% and 3% of cells were viable following exposure to Fmoc-Pyr-DAP for 4 and 24 h, respectively. These higher toxicities may be caused by the strongly lipophilic properties of the perfluorinated benzyl group of Fmoc-F₅-Phe-DAP and the ability of the pyrene group of Pyr-Phe-DAP to intercalate with double-stranded oligonucleotides. Significantly, the remaining N-X-Phe-DAP derivatives exhibited dramatically improved cell viabilities of 40-64% after 48 h; this marks an improvement over Lipofectamine.

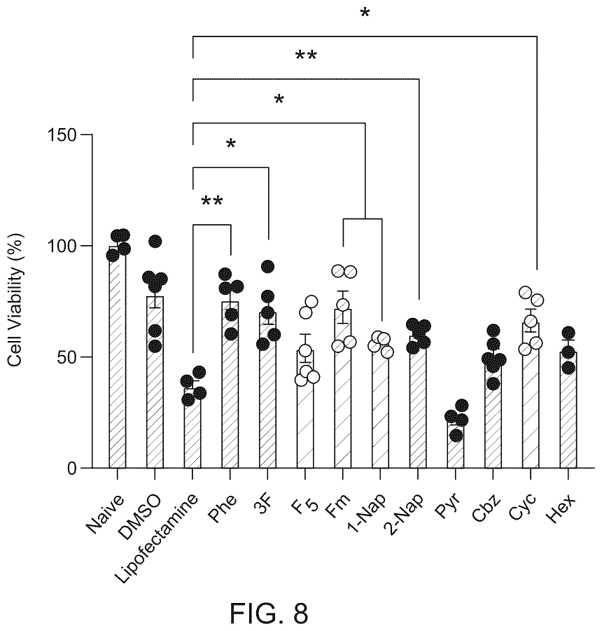

The cells will also recover after exposure to N-X-Phe-DAP derivatives under conditions that resemble knockdown experiments. Cells were dosed with N-X-Phe-DAP derivatives or Lipofectamine for 4 hours, after which the media was aspirated and replaced with DMEM cell culture media and incubated for an additional 48 hours (FIG. 8). This protocol mimics the gene knockdown experiments and acts as a better indication of how cytotoxicity may impact gene silencing measurements. Under these conditions, all of the N-X-Phe-DAP derivatives exhibited significantly improved cell viability (>50%) compared to Lipofectamine (˜44%), with the exception of Pyr-Phe-DAP, which had only ˜22% cell viability. These results are promising and demonstrate that mRNA knockdown is not impacted by cytotoxicity of the Phe derivatives. For example, Cyc-Phe-DAP and Hex-Phe-DAP were relatively cytotoxic (67% and 53% cell viability, respectively), however, the knockdown assay indicated no change in gene expression compared to the naïve control. This data indicates that effective siRNA delivery occurs within the first 4 hours of dosing and that TTF-1 silencing continues for at least 48 hours.

Eight of the ten N-X-Phe-DAP derivatives facilitated efficient siRNA cytosolic delivery and functional mRNA knockdown that matched or surpassed the performance of the often-used delivery agent, Lipofectamine. Only Cyc-Phe-DAP and Hex-Phe-DAP showed less efficient knockdown than Lipofectamine; these derivatives are the only reagents with non-aromatic N-terminal functionalization. The aromatic 1-Nap-Phe-DAP and 2-Nap-Phe-DAP derivatives showed dramatically improved knockdown relative to Lipofectamine controls, with the other aromatic N-X-Phe-DAP derivatives showing similar or slightly improved knockdown relative to Lipofectamine. In general knockdown improves in the order Hex-Phe-DAP<Cyc-Phe-DAP<<Fmoc-3F-Phe-DAP<Fmoc-F₅-Phe-DAP≈Fm-Phe-DAP≈Pyr-Phe-DAP≈Cbz-Phe-DAP≈Fmoc-Phe-DAP<1-Nap-Phe-DAP≈2-Nap-Phe-DAP. Generally, these trends can used to group these reagents in three groups in terms of knockdown efficacy: highly effective knockdown (dramatically improved relative to Lipofectamine; Fmoc-Phe-DAP, 1-Nap-Phe-DAP, 2-Nap-Phe-DAP), effective knockdown (slightly improved or similar relative to Lipofectamine; Fmoc-F₅-Phe-DAP, Fm-Phe-DAP, Pyr-Phe-DAP, Cbz-Phe-DAP), ineffective knockdown (less effective than Lipofectamine; Hex-Phe-DAP<Cyc-Phe-DAP).

These results do not strongly correlate to K_{d} binding affinity values of the N-X-Phe-DAP for siRNA. These reagents can also be arbitrarily divided into three classes of siRNA binders based on K_{d} values: strongly binding (<100 μM; Pyr-Phe-DAP, Cbz-Phe-DAP) moderately binding (100-200 μM; Fmoc-3F-Phe-DAP, Fmoc-Phe-DAP, Cyc-Phe-DAP, Hex-Phe-DAP, 2-Nap-Phe-DAP) and weakly binding (>200 μM; Fmoc-F₅-Phe-DAP, 1-Nap-Phe-DAP, Fm-Phe-DAP). The derivatives with the strongest siRNA binding affinity (Pyr-Phe-DAP, Cbz-Phe-DAP) are in the moderate group of derivatives in terms of knockdown efficiency. The weakest binding group of derivatives includes derivatives that are among the most efficient (1-Nap-Phe-DAP) and among the less efficient (Fmoc-F₅-Phe-DAP) knockdown facilitators. Thus, binding affinity and functional gene silencing do not appear to be directly correlated based on the data. Knockdown efficiency appears to be more closely correlated with the morphology of the condensed N-X-Phe-DAP/siRNA nanoparticles. Nanoparticles that adopt spherical micelle-like morphologies (Fmoc-Phe-DAP, Fm-Phe-DAP, 1-Nap-Phe-DAP, 2-Nap-Phe-DAP) tend to provide more efficient knockdown than those particles that are either highly condensed (Cbz-Phe-DAP or Cyc-Phe-DAP) or which have evident fibrillar structures in addition to spherical micelles (Fmoc-F₅-Phe-DAP, Pyr-Phe-DAP, Hex-Phe-DAP). Collectively, these results suggest that there is a complex relationship between the nature of the N-terminus of N-X-Phe-DAP derivatives, siRNA binding, and functional cytosolic delivery. They also indicate that N-X-Phe-DAP derivatives that have lipid-like assembly properties function better as siRNA delivery agents.

Finally, these studies suggest that N-X-Phe-DAP derivatives that are modified with functional groups that strongly intercalate double-stranded oligonucleotides may have cytotoxic properties that would preclude their use for translational applications. The strong binding of intercalating agents with the DNA double-helix has been shown to induce DNA damage leading to cell death. Specifically, pyrene-modifications have been employed in the development of a variety of anti-cancer drugs. The cytotoxicity of Pyr-Phe-DAP is most likely due to the N-terminal pyrene modification promoting off-target DNA damage in the context of cell-based experiments. The toxicity of the Fmoc-F₅-Phe-DAP derivative may also be due to an increased propensity of the perfluorobenzene side chain to interact with nucleobases through aromatic stacking interactions, which are enhanced by the inverted quadrupole moment of the fluorinated aromatic group.

The disclosed LMW amino acid derivatives demonstrated functional siRNA delivery that mimics the delivery capacity of cell penetrating peptides. The N-X-Phe-DAP derivatives reported herein provide significant advantages over existing siRNA delivery agents, including excellent biocompatibility, low-cost of production, and a general scaffold structure that has great potential for further modification to optimize these agents for in vitro and in vivo siRNA delivery applications. Key design elements are disclosed for inexpensive LMW siRNA delivery reagents, including aromatic/hydrophobic and cationic functionality. In addition, effective LMW delivery agents should generally have supramolecular assembly properties that tend towards lipid-like micelle assembly as opposed to assembly into one-dimensional fibril-like materials.

### VI. EXAMPLES

General Procedure: The preparation of Fmoc-Phe-DAP, Fmoc-3F-Phe-DAP, and Fmoc-F₅-Phe-DAP is described in: Rajbhandary, A.; Raymond, D. M.; Nilsson, B. L., Self-Assembly, Hydrogelation, and Nanotube Formation by Cation-Modified Phenylalanine Derivatives. Langmuir 2017, 33 (23), 5803-5813; Abraham, B. L.; Liyanage, W.; Nilsson, B. L., Strategy to Identify Improved N-Terminal Modifications for Supramolecular Phenylalanine-Derived Hydrogelators. Langmuir 2019, 35 (46), 14939-14948; Abraham, B. L.; Toriki, E. S.; Tucker, N. D. J.; Nilsson, B. L., Electrostatic interactions regulate the release of small molecules from supramolecular hydrogels. J. Mater. Chem. B 2020, 8 (30), 6366-6377; and/or Raymond, D. M.; Abraham, B. L.; Fujita, T.; Watrous, M. J.; Toriki, E. S.; Takano, T.; Nilsson, B. L., Low-Molecular-Weight Supramolecular Hydrogels for Sustained and Localized in Vivo Drug Delivery. ACS Appl. Bio. Mater. 2019, 2 (5), 2116-2124.

The synthesis of other N-X-Phe-DAP derivatives were performed using the strategy outlined in FIG. 9. The first coupling reaction to modify the N-terminus and the following saponification reaction were carried out using previously reported methods. See Abraham, B. L.; Liyanage, W.; Nilsson, B. L., Strategy to Identify Improved N-Terminal Modifications for Supramolecular Phenylalanine-Derived Hydrogelators. Langmuir 2019, 35 (46), 14939-14948. The second coupling reaction and deprotection were performed utilizing another previously reported synthetic method. See Abraham, B. L.; Toriki, E. S.; Tucker, N. D. J.; Nilsson, B. L., Electrostatic interactions regulate the release of small molecules from supramolecular hydrogels. J. Mater. Chem. B 2020, 8 (30), 6366-6377.

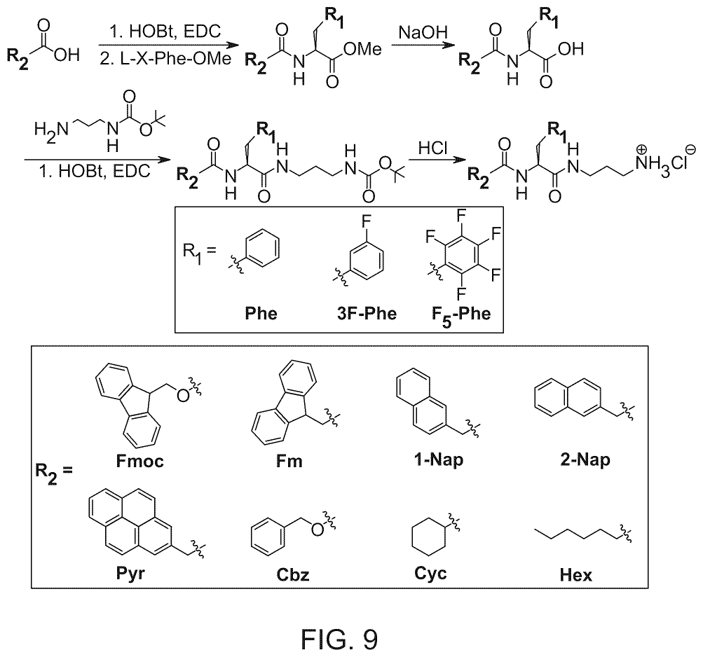

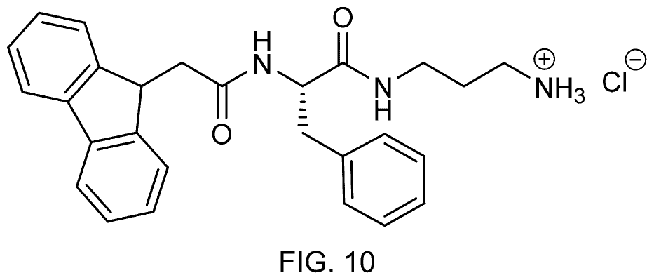

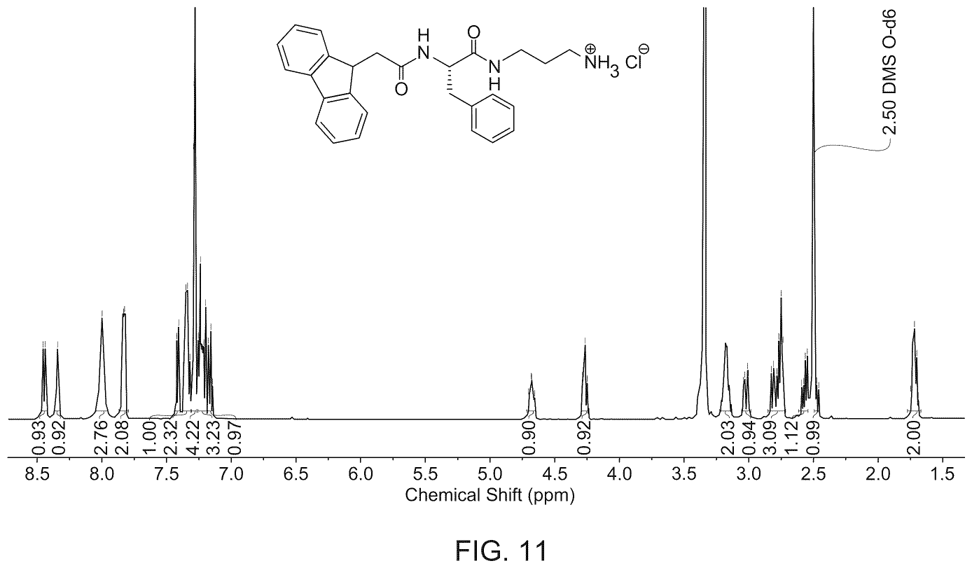

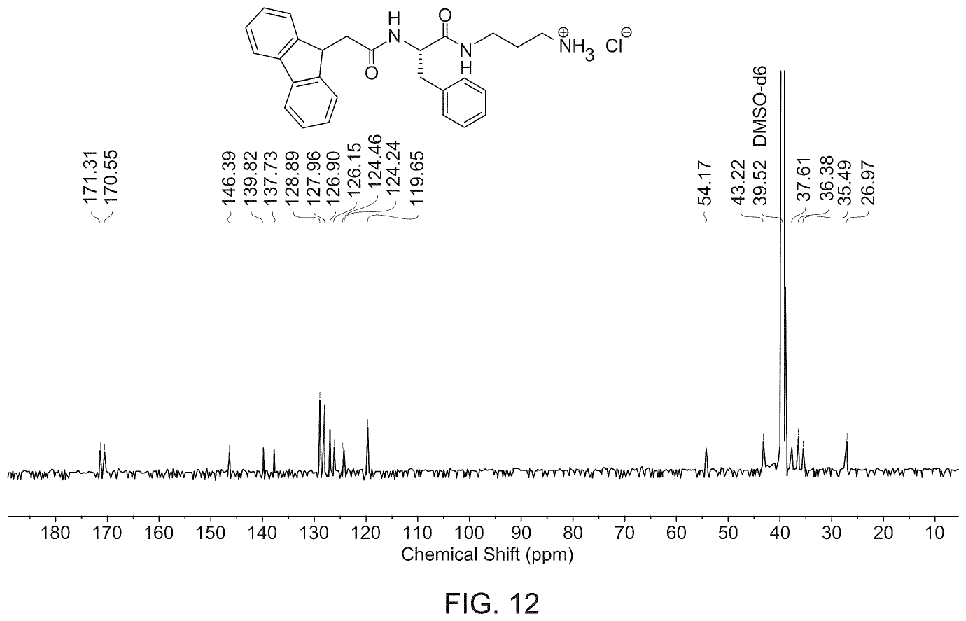

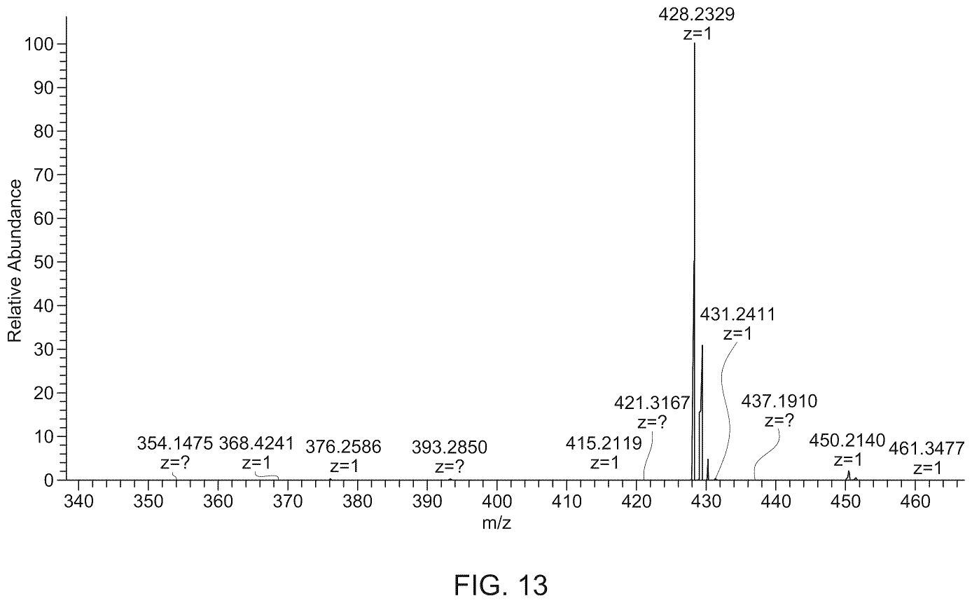

Fm-Phe-DAP (FIG. 10): The product was prepared by following the same general procedure, with 9-fluoreneacetic acid (563 mg, 2.51 mmol) as the N-terminal modifying group. The desired product was obtained as a white powder (486 mg, 1.05 mmol, 42% yield). The product is characterized by: ¹H NMR (500 MHz, DMSO-d6) δ 8.44 (d, J=8.3 Hz, 1H), 8.35 (t, J=5.8 Hz, 1H), 8.00 (s, 3H), 7.83 (dd, J=7.7, 3.1 Hz, 2H), 7.42 (d, J=7.5 Hz, 1H), 7.34 (q, J=7.1 Hz, 2H), 7.29 (dd, J=4.4, 1.0 Hz, 4H), 7.26-7.19 (m, 3H), 7.18-7.14 (m, 1H), 4.68 (td, J=9.1, 5.1 Hz, 1H), 4.27 (t, J=7.5 Hz, 1H), 3.18 (qd, J=6.8, 2.9 Hz, 2H), 3.02 (dd, J=13.7, 5.1 Hz, 1H), 2.85-2.71 (m, 3H), 2.57 (dd, J=14.8, 7.2 Hz, 1H), 2.47 (d, J=7.9 Hz, 1H), 1.72 (p, J=7.0 Hz, 2H) ppm (see FIG. 11); and ¹³C NMR (126 MHz, DMSO-d6) δ 171.31, 170.55, 146.39, 139.82, 137.73, 128.89, 127.96, 126.90, 126.15, 124.46, 124.24, 119.65, 54.17, 43.22, 37.61, 36.38, 35.49, 26.97.ppm (see FIG. 12). FIG. 13 shows the high resolution mass spectrum of Fm-Phe-DAP: HRMS (ESI-TOF) (m/z) 428.2329 (428.2333 calcd for C27H30N3O2 [M]+).

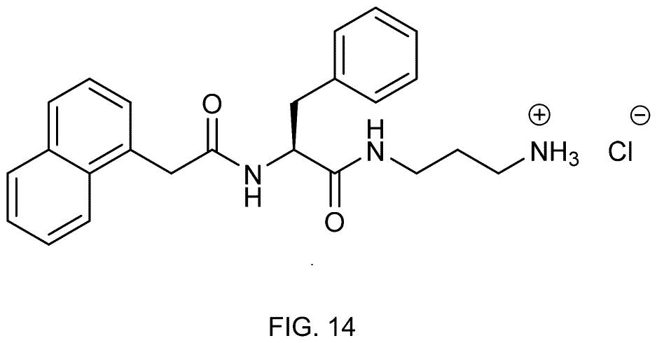

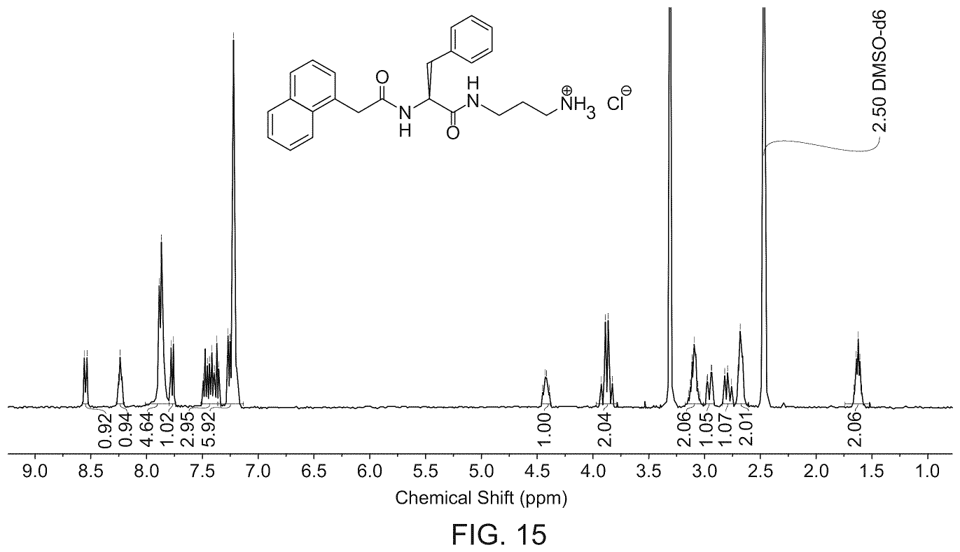

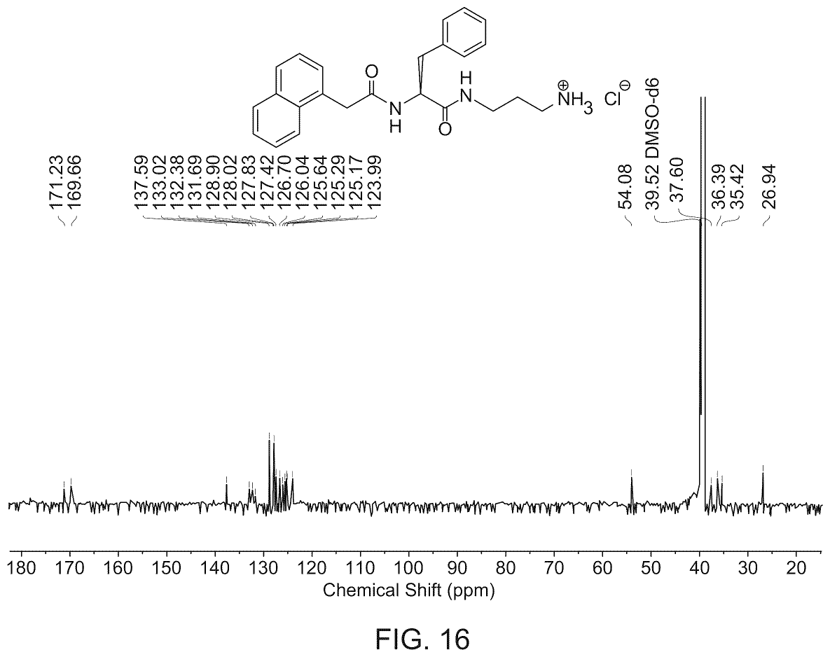

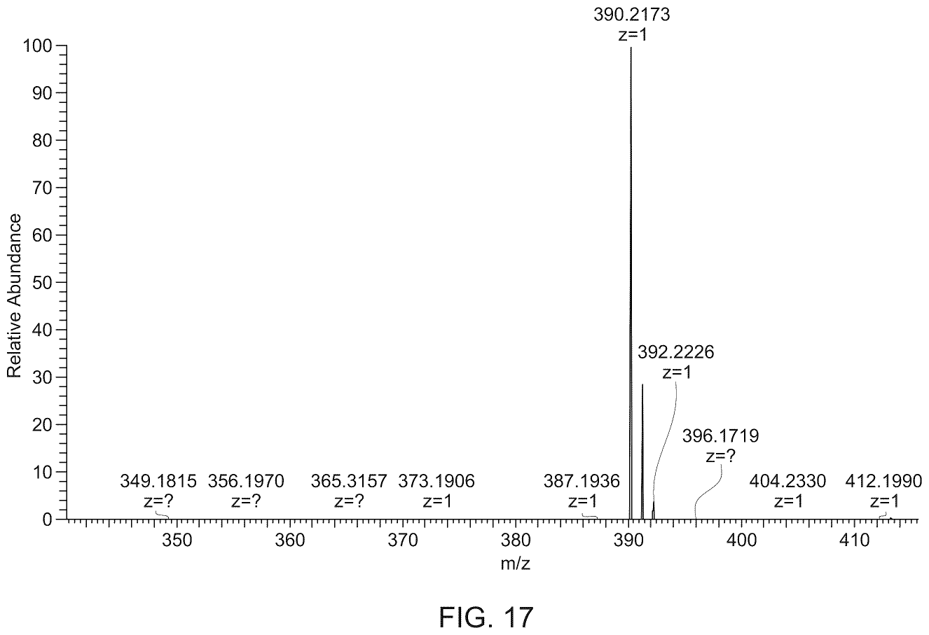

1-Nap-Phe-DAP (FIG. 14): The product was prepared by following the same general procedure, with 1-naphthaleneacetic acid (934 mg, 5.02 mmol) as the N-terminal modifying group. The desired product was obtained as a white powder (1.00 mmol, 20% yield). The product is characterized by: ¹H NMR (400 MHz, DMSO-d6) δ 8.55 (d, J=8.3 Hz, 1H), 8.28-8.20 (m, 1H), 7.88 (d, J=8.3 Hz, 5H), 7.78 (d, J=8.3 Hz, 1H), 7.52-7.33 (m, 3H), 7.31-7.16 (m, 6H), 4.48-4.40 (m, 1H), 3.98-3.83 (m, 2H), 3.19-3.06 (m, 2H), 2.98 (dd, J=13.7, 4.9 Hz, 1H), 2.87-2.76 (m, 1H), 2.71 (t, J=7.6 Hz, 2H), 1.72-1.61 (m, 2H) ppm (see FIG. 15); and ¹³C NMR (126 MHz, DMSO-d6) δ 171.23, 169.66, 137.59, 133.02, 132.38, 131.69, 128.90, 128.02, 127.83, 127.42, 126.70, 126.04, 125.64, 125.29, 125.17, 123.99, 54.08, 37.60, 36.39, 35.42, 26.94 ppm (see FIG. 16). FIG. 17 shows the high resolution mass spectrum of 1-Nap-Phe-DAP: HRMS (ESI-TOF) (m/z) 390.2127 (390.2176 calcd for C24H28N3O2[M]+).

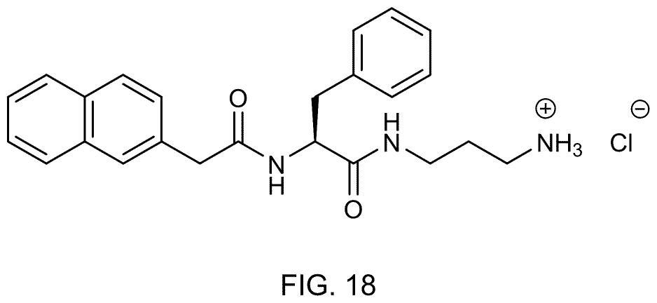

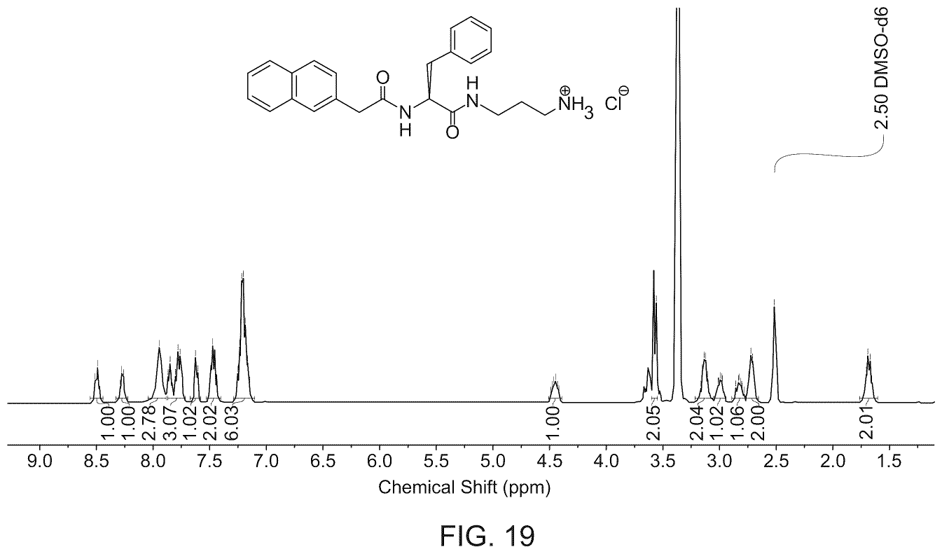

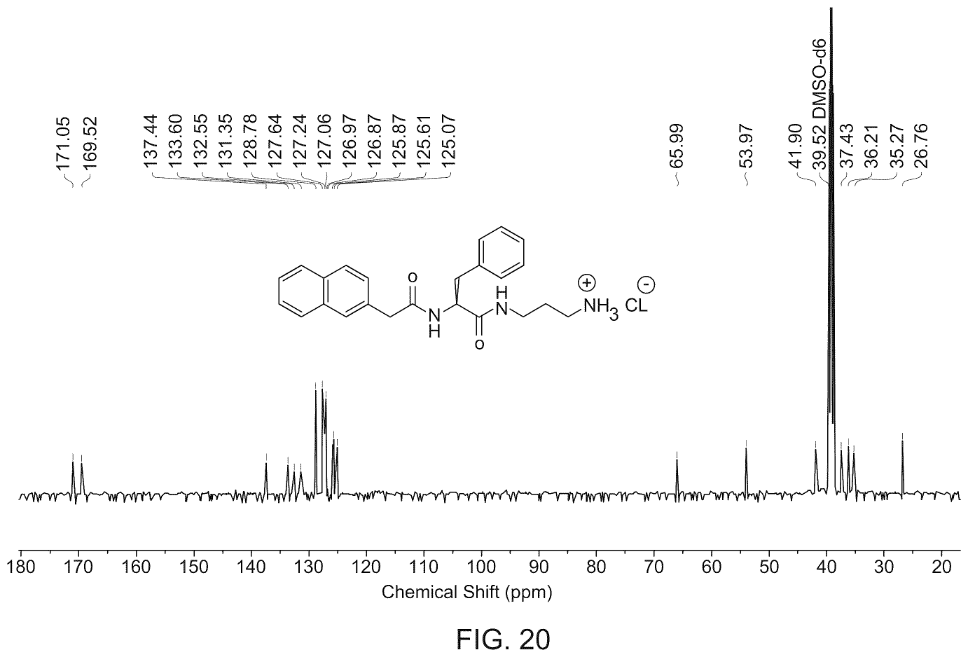

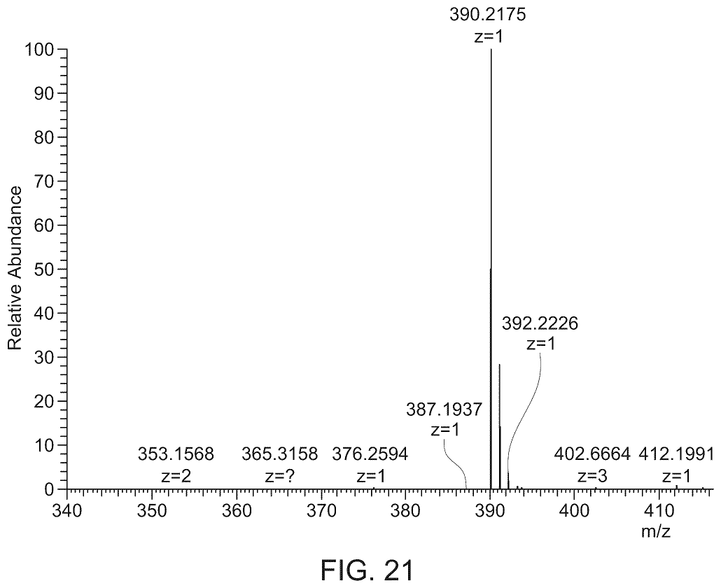

2-Nap-Phe-DAP (FIG. 18): The product was prepared by the general procedure, with 2-naphthaleneacetic acid (749 mg, 4.02 mmol) as the N-terminal modifying group. The desired product was obtained as a white powder (159 mg, 1.77 mmol, 9.3% yield). The product is characterized by: 1H NMR (400 MHz, DMSO-d6) δ 8.50 (d, J=7.8 Hz, 1H), 8.27 (t, J=6.3 Hz, 1H), 7.94 (s, 3H), 7.87-7.72 (m, 3H), 7.61 (d, J=6.8 Hz, 1H), 7.52-7.40 (m, 2H), 7.28-7.12 (m, 6H), 4.49-4.38 (m, 1H), 3.58-3.54 (m, 2H), 3.18-3.06 (m, 2H), 3.03-2.94 (m, 1H), 2.86-2.75 (m, 1H), 2.71 (t, J=7.4 Hz, 2H), 1.73-1.61 (m, 2H) ppm (see FIG. 19); and ¹³C NMR (126 MHz, DMSO-d6) δ 171.05, 169.52, 137.44, 133.60, 132.55, 131.35, 128.78, 127.64, 127.24, 127.06, 126.97, 126.87, 125.87, 125.61, 125.07, 65.99, 53.97, 41.90, 37.43, 36.21, 35.27, 26.76 ppm (see FIG. 20). FIG. 21 shows the high resolution mass spectrum of 2-Nap-Phe-DAP: HRMS (ESI-TOF) (m/z) 390.2176 (390.2176 calcd for C24H28N3O2[M]+).

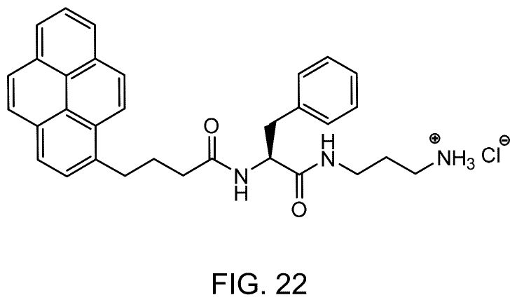

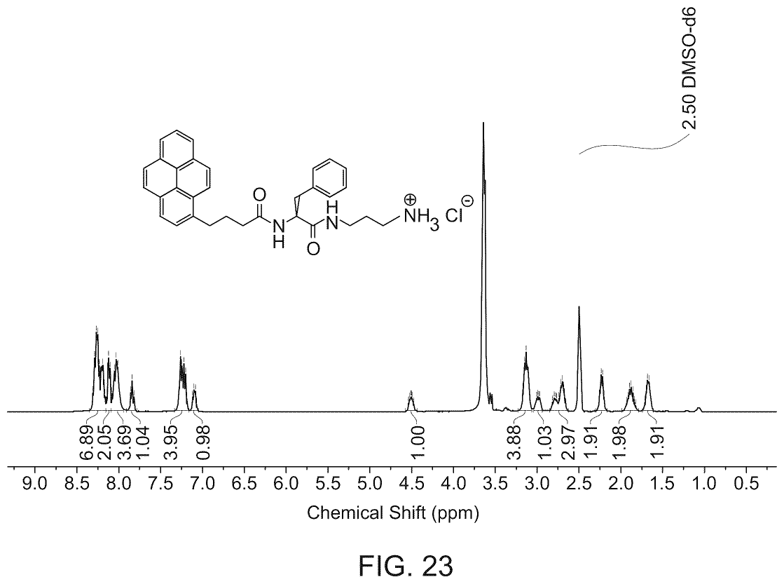

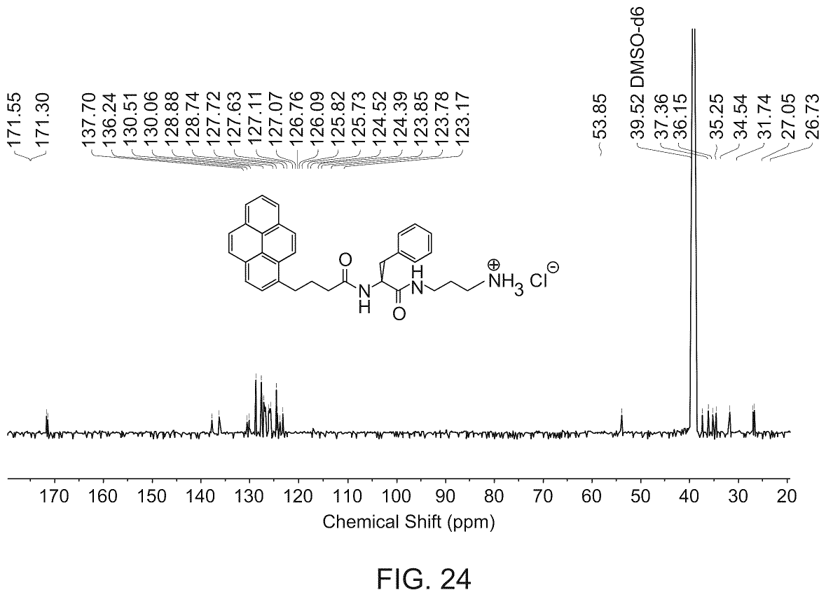

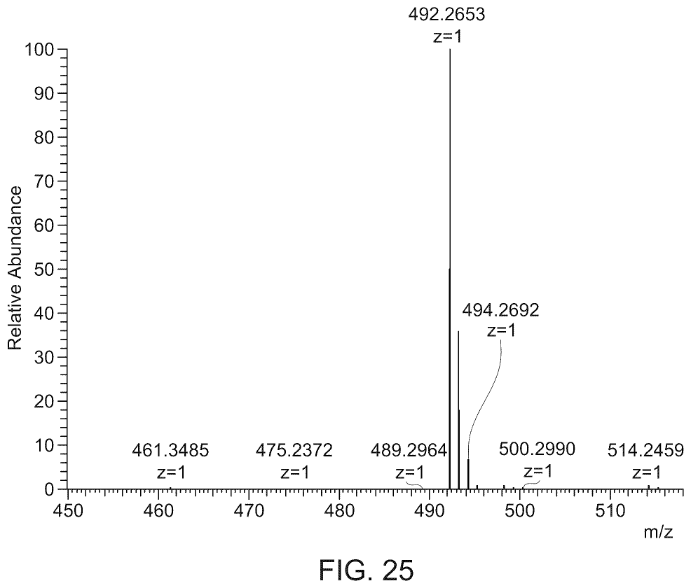

Pyr-Phe-DAP (FIG. 22): The product was prepared by the general procedure, with 1-pyrenebutyric acid (1445 mg, 5.01 mmol) as the N-terminal modifying group. The desired product was obtained as a white powder (419 mg, 1.77 mmol, 16% yield). The product is characterized by: ¹H NMR (400 MHz, DMSO-d6) δ 8.35-8.15 (m, 7H), 8.16-8.09 (m, 2H), 8.09-7.96 (m, 4H), 7.84 (t, J=7.7 Hz, 1H), 7.23 (dq, J=14.6, 7.5 Hz, 4H), 7.15-7.02 (m, 1H), 4.59-4.44 (m, 1H), 3.14 (q, J=7.8 Hz, 4H), 2.98 (dd, J=10.3, 5.0 Hz, 1H), 2.86-2.60 (m, 3H), 2.23 (q, J=7.3 Hz, 2H), 1.89 (dq, J=14.2, 6.7 Hz, 2H), 1.67 (q, J=6.8 Hz, 2H) ppm (see FIG. 23); and ¹³C NMR (126 MHz, DMSO-d6) δ 171.55, 171.30, 137.70, 136.24, 130.51, 130.06, 128.88, 128.74, 127.72, 127.63, 127.11, 127.07, 126.76, 126.09, 125.82, 125.73, 124.52, 124.39, 123.85, 123.78, 123.17, 53.85, 37.36, 36.15, 35.25, 34.54, 31.74, 27.05, 26.73 ppm (see FIG. 24). FIG. 25 shows the high resolution mass spectrum of Pyr-Phe-DAP: HRMS (ESI-TOF) (m/z) 492.2653 (492.2646 calcd for C32H34N3O2 [M]+).

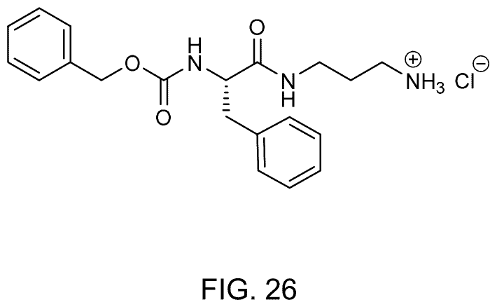

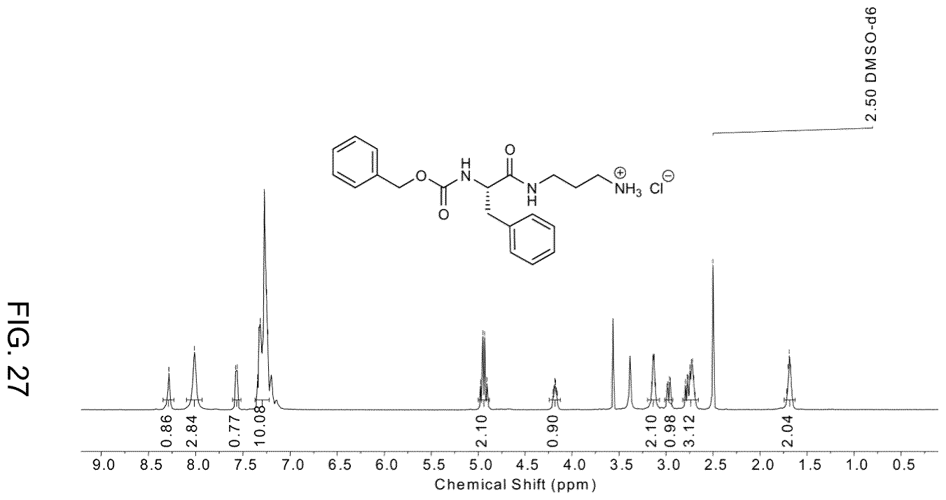

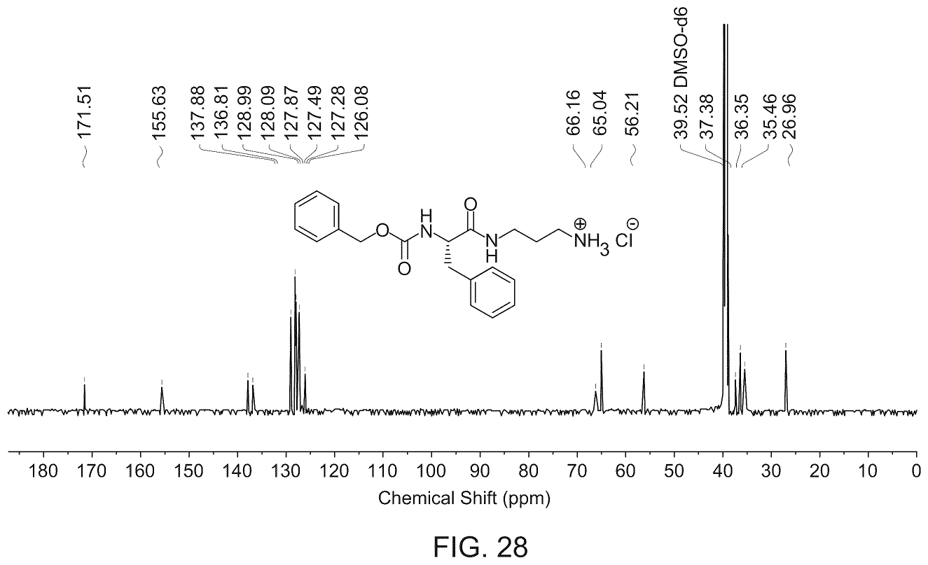

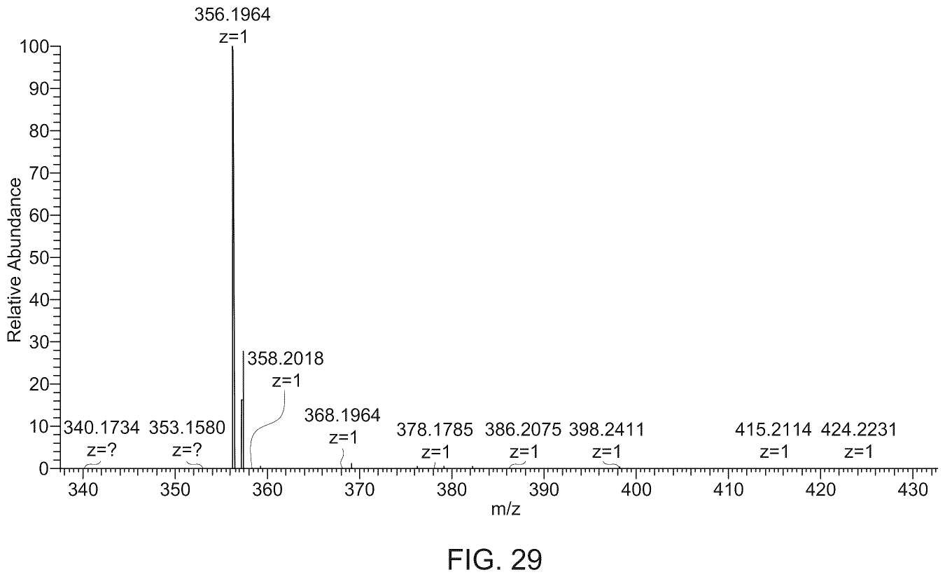

Cbz-Phe-DAP (FIG. 26): The product was prepared by the general procedure, with N-carbobenzloxy-L-phenylalanine (772 mg, 2.58 mmol). The desired product was obtained as a white powder (732 mg, 1.87 mmol, 72% yield). The product is characterized by: ¹H NMR (500 MHz, DMSO-d6) δ 8.28 (s, 1H), 8.01 (s, 3H), 7.57 (d, J=8.4 Hz, 1H), 7.38-7.17 (m, 10H), 4.94 (q, J=12.7 Hz, 2H), 4.18 (td, J=9.5, 4.7 Hz, 1H), 3.13 (dt, J=9.5, 6.7 Hz, 2H), 2.97 (dd, J=13.6, 4.7 Hz, 1H), 2.74 (ddd, J=27.3, 13.2, 8.2 Hz, 3H), 1.69 (p, J=7.0 Hz, 2H) ppm (see FIG. 27); and ¹³C NMR (126 MHz, DMSO-d6) δ 171.51, 155.63, 137.88, 136.81, 128.99, 128.09, 127.87, 127.49, 127.28, 126.08, 66.16, 65.04, 56.21, 37.38, 36.35, 35.46, 26.96 ppm (see FIG. 28). FIG. 29 shows the high resolution mass spectrum of Cbz-Phe-DAP: HRMS (ESI-TOF) (m/z) 356.1964 (356.1969 calcd for C20H26N3O3 [M]+).

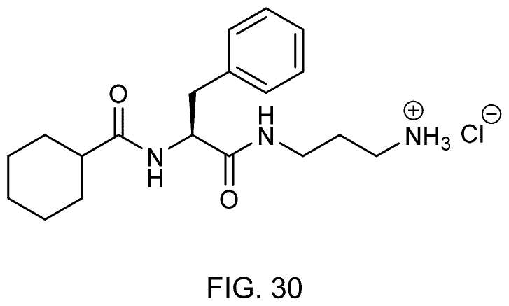

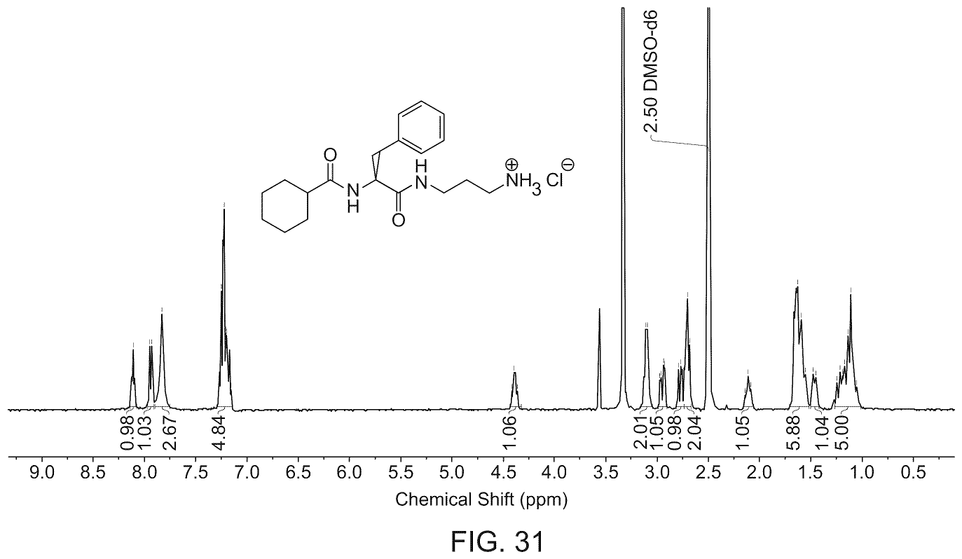

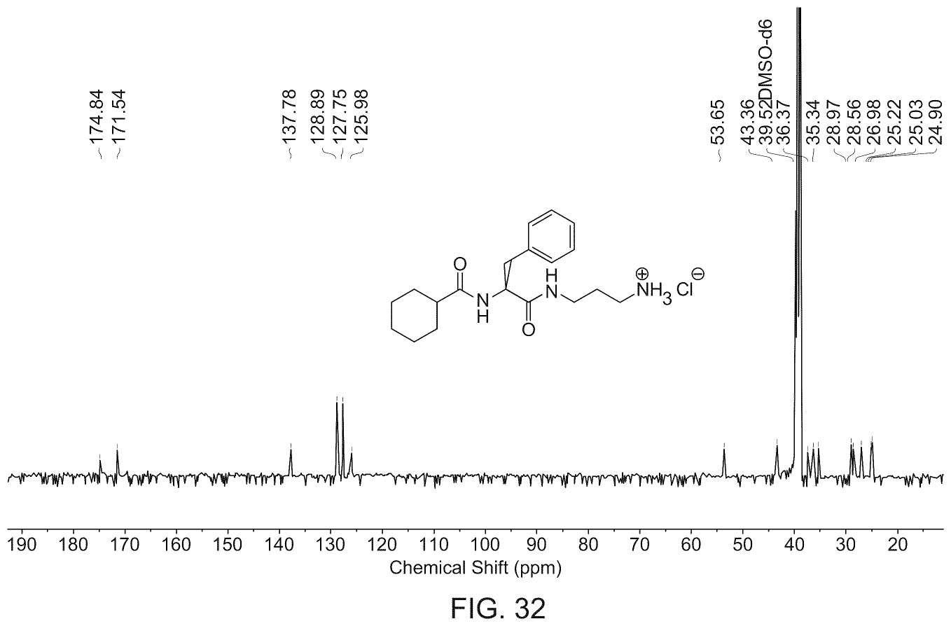

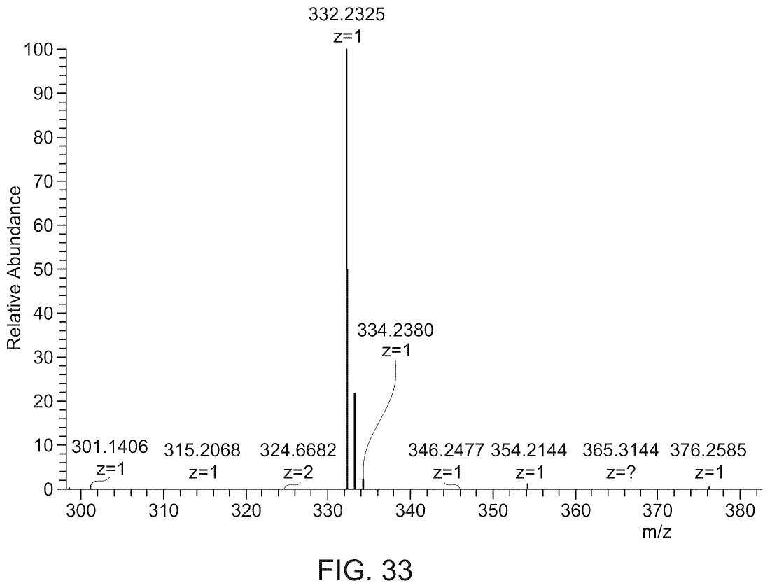

Cyc-Phe-DAP (FIG. 30): The product was prepared according to the general procedure, with cyclohexane carboxylic acid (643 mg, 5.02 mmol) as the N-terminal modifying group. The desired product was obtained as a white powder (84 mg, 0.23 mmol, 4.6% yield). The product is characterized by: ¹H NMR (400 MHz, DMSO-d6) δ 8.11 (t, J=5.7 Hz, 1H), 7.94 (d, J=8.2 Hz, 1H), 7.83 (s, 3H), 7.29-7.13 (m, 5H), 4.43-4.33 (m, 1H), 3.10 (d, J=6.6 Hz, 2H), 2.95 (dd, J=13.6, 5.0 Hz, 1H), 2.81-2.74 (m, 1H), 2.71 (q, J=6.7 Hz, 2H), 2.11 (t, J=10.8 Hz, 1H), 1.70-1.52 (m, 6H), 1.47 (d, J=10.4 Hz, 1H), 1.28-1.02 (m, 5H) ppm (see FIG. 31); and ¹³C NMR (126 MHz, DMSO) δ 174.84, 171.54, 137.78, 128.89, 127.75, 125.98, 53.65, 43.36, 37.41, 36.37, 35.34, 28.97, 28.56, 26.98, 25.22, 25.03, 24.90 ppm (see FIG. 32). FIG. 33 shows the high resolution mass spectrum of Cyc-Phe-DAP: HRMS (ESI-TOF) (m/z) 332.2325 (332.2333 calcd for C19H30N3O2 [M]+).

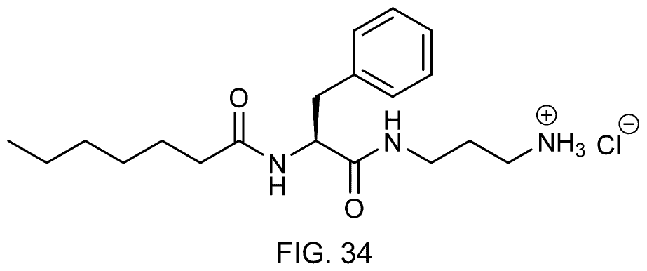

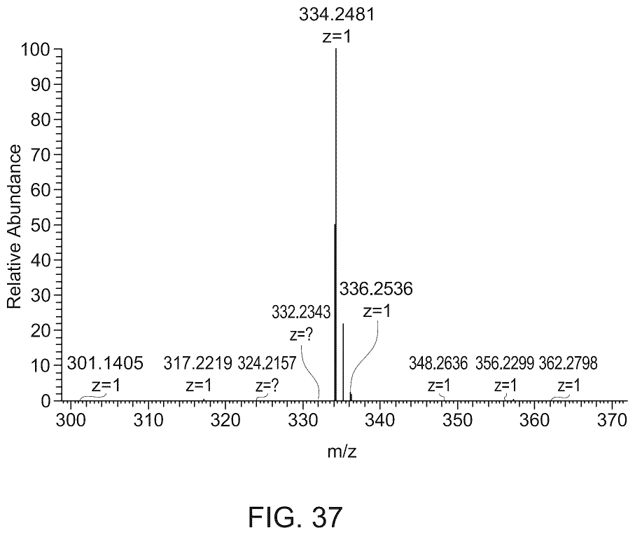

Hex-Phe-DAP (FIG. 34): The product was prepared by following the general procedure, with heptanoic acid (653 mg, 5.02 mmol) as the N-terminal modifying group. The desired product was obtained as a white powder (119.3 mg, 0.32 mmol, 6.4% yield). The product is characterized by: ¹H NMR (400 MHz, DMSO-d6) δ 8.18 (t, J=5.8 Hz, 1H), 8.09 (d, J=8.4 Hz, 1H), 7.93 (s, 3H), 7.29-7.20 (m, 4H), 7.20-7.14 (m, 1H), 4.46-4.37 (m, 1H), 3.16-3.05 (m, 2H), 2.95 (dd, J=13.6, 5.0 Hz, 1H), 2.80-2.65 (m, 3H), 2.03 (t, J=7.4 Hz, 2H), 1.71-1.61 (m, 2H), 1.40-1.28 (m, 2H), 1.25-1.02 (m, 6H), 0.87-0.78 (m, 3H) ppm (see FIG. 35); and ¹³C NMR (126 MHz, DMSO-d6) δ 171.68, 171.27, 137.63, 128.68, 127.58, 125.79, 53.71, 37.31, 36.16, 35.19, 34.83, 30.59, 27.74, 26.73, 24.69, 21.53, 13.50 ppm (see FIG. 36). FIG. 37 shows the high resolution mass spectrum of Hex-Phe-DAP: HRMS (ESI-TOF) (m/z) 334.2481 (334.2489 calcd for C19H32N3O2 [M]+).

Dynamic Light Scattering: N-X-Phe-DAP derivatives (100 μM) were complexed with siRNA (600 nM) in Ultrapure™ DNase/RNase-Free Distilled Water (Invitrogen 10977015) and allowed to incubate for 30 minutes at room temperature. These solutions were then diluted into water to give final concentrations of 5 μM Phe derivative and 30 nM siRNA. Dynamic light scattering measurements were taken using a DynaPro Plate Reader II. Diameter is reported as an average of 10 measurements using Dynamics software.

Transmission Electron Microscopy: N-X-Phe-DAP derivatives (100 μM) were complexed with siRNA (600 nM) in Ultrapure™ DNase/RNase-Free Distilled Water (Invitrogen 10977015) and allowed to incubate for 30 minutes at room temperature. These solutions were then diluted into water to give final concentrations of 5 μM Phe derivative and 30 nM siRNA. Samples (5 μL) were spotted on 200 mesh carbon coated copper grids and excess solvent was removed by capillary action with filter paper after 60 seconds. Grids were then stained with uranyl acetate (5 μL) for 60 seconds by excess solvent was removed by capillary action with filter paper. Grids were left to dry for 10 minutes. Images were taken using a Hitachi 7650 transmission electron microscope with an accelerating voltage of 80 kV.

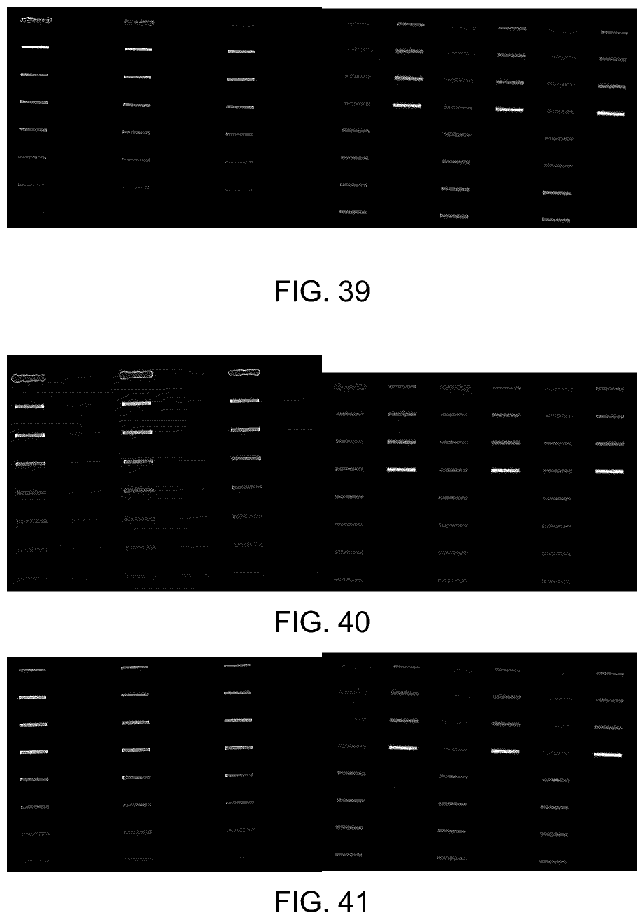

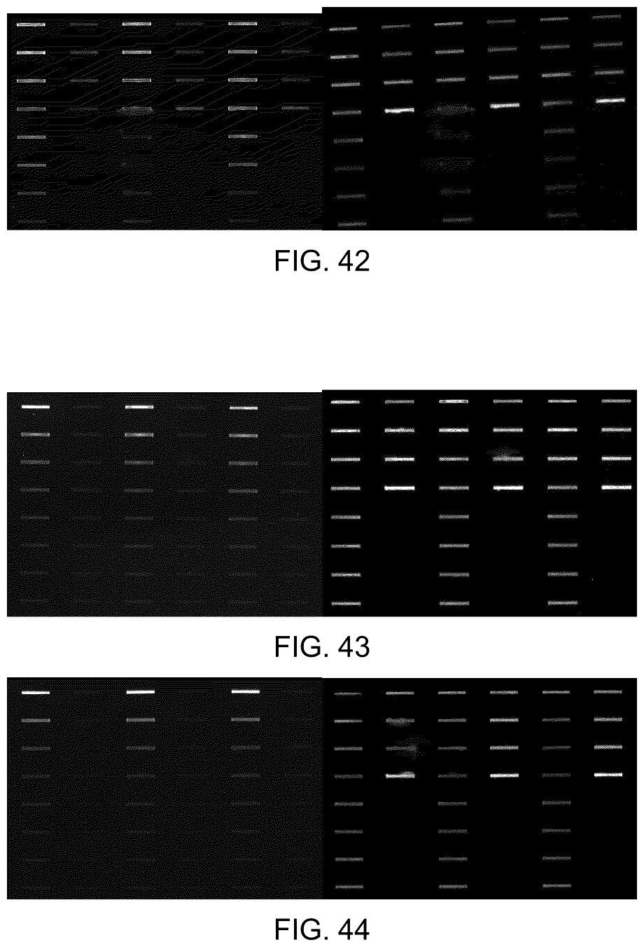

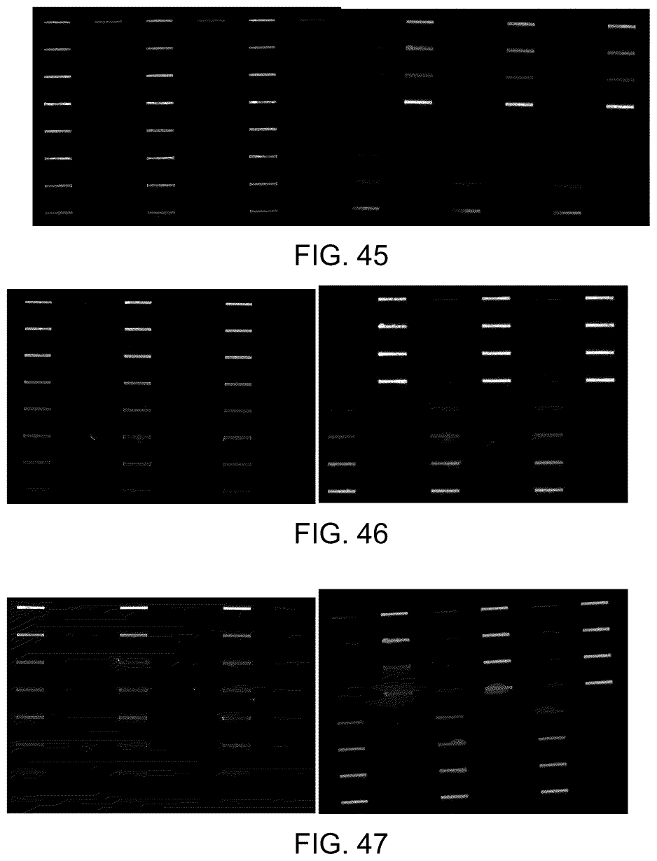

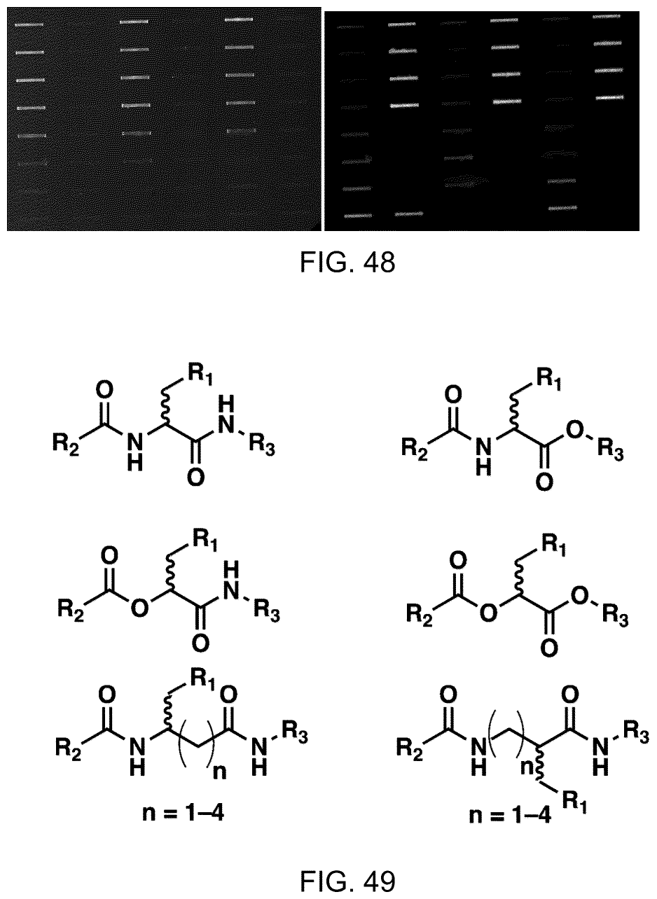

Slot Blot Binding Affinity Measurements (see FIGS. 39-48): Nitrocellulose (Bio-Rad, #1620112) and nylon membranes (VWR, #95038-400) were soaked in 100 mM Tris-HCl buffer for 20 minutes. The membranes were then stacked on two sheets of Bio-Rad SF thick filter paper (#1620161) in a Bio-Rad SF 48-well slot blot apparatus. 200 μL of 100 mM Tris-HCl buffer was added to each well to test for even vacuum distribution and for membrane rehydration three times. N-X-Phe-DAP derivatives were prepared in concentrations ranging from 4000 μM to 0 μM and incubated with FITC-siRNA (6.5 μL, 1 μM) for 30 minutes in the dark. 40 μL of each sample, in addition to 200 μL Tris-HCl buffer was run in triplicate and the apparatus was covered with aluminum foil for 20 minutes. The vacuum was opened, allowing for samples in the wells to penetrate the membranes. After closing the vacuum, 200 μL of Tris-HCl buffer was added to rinse each well followed by re-opening of the vacuum three times. Membranes were then dried and imaged on Bio-Rad ChemiDoc MS. Fluorescence intensity density measurements were made using Image J. The fraction bound of siRNA was calculated using the following equation, where Iₙᵢₜᵣₒ is the fluorescence intensity measured from the nitrocellulose membrane and I_{nylon} is the fluorescence intensity measured from the nylon membrane:

$$Fraction Bound = \frac{{I}_{n i t r o}}{{I}_{n i t r o} + {I}_{nylon}}$$

Binding affinities along with Hill slopes and Bₘₐₓ values were extrapolated by analyzing this data in GraphPad Prism using a built-in specific binding with Hill slope nonlinear regression. This equation is as follows where Y is the fraction bound, X is the N-X-Phe-DAP concentration (μM), Bₘₐₓ is the maximum binding, K_{d} is the binding affinity, and h is the Hill slope:

$$Y = \frac{{B}_{max} {X}^{h}}{{K}_{d}^{h} + {X}^{h}}$$

Sample Preparation for Cell-Based Experiments: For delivery analysis and functional knockdown, N-X-Phe-DAP derivatives (10 μL, 1 mM) or Lipofectamine 3000 (6 μL) was mixed with TTF-1 siRNA (Santa Cruz Biotechnology sc-36756, 6 μL, 10 μM) in Ultrapure™ DNase/RNase-Free Distilled Water (Invitrogen 10977015) to give a final volume of 100 μL. These mixtures were incubated for 30 minutes at room temperature, followed by dilution into 1×DMEM (Gibco 11965092, 1.9 mL). For cytotoxicity assays, N-X-Phe-DAP derivatives (10 μL, 1 mM) were diluted into 1×DMEM (Gibco 11965092, 1.9 mL).

N-X-Phe-DAP-siRNA Cellular Delivery Analysis A549 lung adenocarcinoma cells (ATCC #) were seeded at a density of 1.05×10⁵ cells/well in a glass bottom 24-well plate (500 μL) and grown to 70-90% confluency. Cells were washed with 1× phosphate buffered saline (PBS, Gibco 14190144), samples were added (500 μL/well), and incubated for 4 h at 37° C., 5% C02. After 4 h, media was removed, cells were washed with 1×PBS, and fixed with 4% paraformaldehyde solution. After 10 min, cells were washed twice with 1×PBS and incubated with DAPI stain (1 μg/mL) in the dark. After 5 min, cells were washed three times with 1×DPBS and stored at −20° C. until imaging. A Nikon T1 epifluorescence microscope was used for fluorescent imaging. Images were collected at 40× magnification using NIS-Elements Viewer (Nikon) software. All images were processed in Adobe Photoshop by selecting “image” then “Adjustments” then “Levels”. In the pop-up window, “Channel” was selected followed by “Green” and Output levels were adjusted with an upper limit of 200. In the sample pop-up window under “Options” the “Enhance Per Channel Contrast” was selected under algorithms.

TTF-1 Knockdown Determination: All knockdown experiments were performed in triplicate. A549 cells were seeded at a density of 1.05×10⁵ cells/well in a 96-well plate (200 μL) and grown to 70-90% confluency. Cells were washed twice with 1×PBS (200 μL/well), N-X-Phe-DAP/siRNA samples were added (200 μL/well), and mixtures were incubated for 4 h at 37° C., 5% CO2. After 4 h, the media was removed, cells were washed with 1×PBS, wells were replenished with complete media containing 1×DMEM (Gibco 11965092), 10% fetal bovine serum (FBS, Invitrogen 26140087), and 1× antibiotic-antimycotic (Gibco 15240-062) and incubated an additional 48 h.

Cells were lysed and RNA was extracted using an Aurum Total RNA Mini Kit (Bio-Rad 7326820). In brief, cells were lysed with Lysis solution containing 1% β-mercaptoethanol (200 μL). Lysate was then mixed with 70% EtOH (200 μL) and processed using QIAshredder homogenizer columns (Qiagen 79656). Homogenate was then transferred to Aurum RNA binding columns, washed with Low Stringency Wash Solution, and incubated with diluted DNase I solution (80 μL) for 15 minutes at RT. Samples were then serially washed with High and Low Stringency Wash Solutions and RNA was eluded from the column with Ultrapure™ DNase/RNase-Free Distilled Water (Invitrogen 10977015, 80 μL). Recovered RNA was quantified on a Nanodrop One system and samples were diluted in Ultrapure™ DNase/RNase-Free Distilled Water (Invitrogen 10977015, 2 ng/μL). Relative TTF-1 knockdown was quantified by RT-qPCR

An iScript cDNA Synthesis Kit (Bio-Rad 1708891) was used for reverse transcription of extracted RNA samples. In brief, samples were prepared by combining 5× iScript Reaction Mix (4 μL), iScript Reverse Transcriptase (1 μL), extracted RNA (12.5 μL, 2 ng/μL), and Ultrapure™ DNase/RNase-Free Distilled Water (Invitrogen 10977015, 2.5 μL). Samples were incubated on a Bio-Rad Thermal Cycler using the following protocol: 5 min priming at 25° C., 20 min reverse transcription at 40° C., and 1 min reverse transcriptase inactivation at 95° C. Recovered cDNA was quantified on a Nanodrop One and samples were diluted in Ultrapure™ DNase/RNase-Free Distilled Water (Invitrogen 10977015, 50 ng/μL). Gene expression was quantified utilizing qPCR. A primer stock solution (750 nM, 267 μL, H2O) of TTF-1 (Santa Cruz Biotechnology, sc-36756-PR) or GAPDH (Santa Cruz Biotechnology, sc-35448-PR) primer pairs were prepared by dilution of Primer A (20 μL, 10 μM) and Primer B (20 μL, 10 μM) into Ultrapure™ DNase/RNase-Free Distilled Water (Invitrogen 10977015, 227 μL). Samples were prepared in triplicate by combining 2× iTaq Universal SYBR Green Super Mix (5 μL, Bio-Rad), TTF-1 or GAPDH primer stock (4 μL, 750 nM), and recovered cDNA (1 μL, 50 ng/μL) in sterile qPCR tubes. Quantitative Ct values were obtained by running samples on a Bio-Rad CFX96 Touch using the “PWRUP60” protocol. Gene expression values were calculated by utilizing a variation of the Livak Method, in which GAPDH was used as the reference gene.

MTT Cytotoxicity Assay: All cytotoxicity assays were performed in triplicate. A549 cells were seeded at a density of 2400 cells/well in a 96-well plate (200 μL) and grown to 70-90% confluency. Cells were washed with 1×PBS (200 μL), N-X-Phe-DAP derivatives were added (200 μL/well), and incubated 4 h, 24 h, or 48 h at 37° C., 5% CO₂ or for 4 h at 37° C., 5% CO₂ followed by washing with 1×PBS and addition of complete media containing 1×DMEM (Gibco 11965092), 10% fetal bovine serum (FBS, Invitrogen 26140087), and 1× antibiotic-antimycotic (Gibco 15240-062). A solution of thiazoyl blue tetrazolium bromide (MTT, 1 mg/mL) was prepared in 1×DMEM devoid of phenol red (Invitrogen 31053028). After the appropriate incubation time, cells were washed with 1×PBS, dosed with MTT (200 μL), and incubated an additional 1 h at 37° C., 5% C02 in the dark. The 96-well plate was centrifuged for 5 min at 4000 rpm, the media was aspirated, and DMSO (100 μL) was added to the wells. Absorbance measurements (550 nm) were taken on a Tecan Infinite M1000 Pro plate reader. Cell viability was then calculated and normalized to the naïve control.

### VII. AMINO ACID DERIVATIVES

The N-X-Phe-DAP derivatives disclosed provide significant advantages over existing siRNA delivery agents, including excellent biocompatibility, low-cost of production, and a general scaffold structure that has great potential for further modification to optimize these agents for in vitro and in vivo siRNA delivery applications. One potential modification is the use of derivatives based on amino acids other than phenylalanine. Thus, also disclosed are low molecular weight amino acid derivatives that will condense with siRNA to form nanoparticles that facilitate cytosolic delivery and functional knockdown of target genes. The amino acid derivatives are modified at the N- and C-termini respectively with aromatic/hydrophobic and cationic functional groups that mirror common chemical properties of peptide-based delivery agents. The embodiments of these derivatives are shown in FIG. 49. In FIG. 49, R₁ is a canonical or non-canonical amino acid; R₂ is a hydrophobic, aromatic or charged modifying group; R₃ is hydrophobic, aromatic or charged modifying group; and n is an integer from 1 to 4. A hydrophobic, aromatic or charged modifying group is defined as chemical functional groups that are either aliphatic or aromatic structures and positively charged groups (including ammonium, alkylated ammonium, and guanidium cations) that can be attached to the amino acid core via amide or ester bonds to the N- or C-termini and may include the following species:

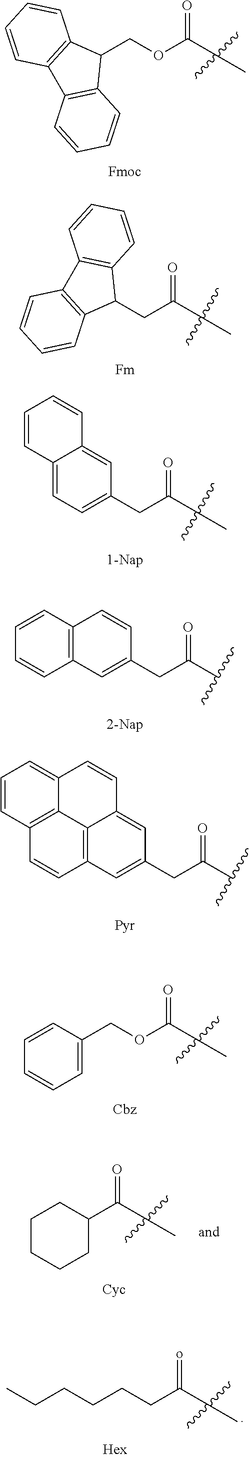

The experiments demonstrating the suitability of N-X-Phe-DAP derivatives as delivery agents demonstrate that other amino acid derivatives would have similar, or better, physical properties and also facilitate functional delivery of siRNA. The amino acid derivatives would have similar molecular weight, diameter and binding affinity (K_{d}) to siRNA as do the N-X-Phe-DAP derivatives. Both aromatic and non-aromatic amino acid derivatives are predicted to interact with siRNA with aromatic character, hydrophobicity, and chemical spatial orientation all having an impact on binding affinity.

Eight of the ten N-X-Phe-DAP derivatives facilitated efficient siRNA cytosolic delivery and functional mRNA knockdown that matched or surpassed the performance of the often-used delivery agent, Lipofectamine. As did the N-X-Phe-DAP derivatives, other amino acid derivatives should form complexes with siRNA and have knockdown properties dependent on their structure. Nanoparticles formed by complexation of siRNA with N-X-Phe-DAP derivatives were shown to form spherical micelle-like structures, fibril-like structures and/or sheet-like structures and mixtures thereof. It has been hypothesized that the knockdown efficiency of N-X-Phe-DAP derivatives does not correlate to K_{d} binding affinity values but instead appears to be more closely correlated with the morphology of the condensed N-X-Phe-DAP/siRNA nanoparticles. Nanoparticles that adopt spherical micelle-like morphologies tend to provide more efficient knockdown than those particles that are either highly condensed or which have evident fibrillar structures in addition to spherical micelles. Factors in knockdown efficiency are believed to include the nature of the N-terminus of N-X-Phe-DAP derivatives, siRNA binding, and functional cytosolic delivery. N-X-Phe-DAP derivatives that have lipid-like assembly properties function better as siRNA delivery agents. Other amino acid derivatives may exhibit similar properties that result in improved knockdown efficiencies or exhibit other properties that result in improved knockdown efficiencies.

In addition, all of the N-X-Phe-DAP derivatives exhibited significantly improved cell viability (>50%) compared to Lipofectamine (˜44%), with the exception of Pyr-Phe-DAP, which had only ˜22% cell viability. These results are promising and demonstrate that mRNA knockdown is not impacted by cytotoxicity of the Phe derivatives. It is predicted that mRNA knockdown will similarly not be impacted by cytotoxicity of other amino acid derivatives.

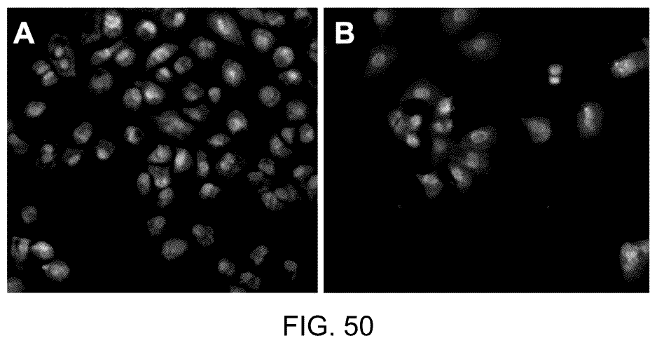

Cells treated with a mixture of 1-Nap-Phe-DAP showed that the oligonucleotides were delivered into the cell. FIG. 50 has images of cells treated with a mixture of 1-Nap-Phe-DAP with fluorescent protein encoding mRNA (panel A) and plasmid DNA (panel B). These images show fluorescence visible in the cytosol, demonstrating that the oligonucleotides were delivered. In this example, 14 nM of the 1-Nap-Phe-DAP was used with 1 microgram of mRNA or pDNA for 24 hours, after which the cells were images.

### VIII. CELL DELIVERY METHODS

Disclosed is a method of delivering RNA into a cell, the method comprising: (a) mixing the RNA with one or more amino acid derivatives as disclosed above and of the general structure disclosed in FIG. 49; (b) diluting the mixture into a cell culture medium; and (c) dosing the cell with the diluted mixture. Also disclosed is a method of delivering RNA into a cell, the method comprising: (a) mixing the RNA with one or more phenylalanine derivatives with the formula:

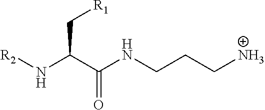

wherein R₁ is phenylalanine or a fluorinated phenylalanine; and R₂ is an N-terminal modifying group; (b) diluting the mixture into a cell culture medium; and (c) dosing the cell with the diluted mixture.

The disclosed methods may be used to deliver various types of oligonucleotides to cells, including mRNA, siRNA (and its variations), miRNA, and/or short hairpin RNA (shRNA), or plasmid DNA. Any type of cell culture medium created to support cellular growth may be used in the method, including natural media, such as biological fluids, including serum, plasma, and lymph fluids, and tissue extracts, and synthetic media such as MEM (Minimum Essential Medium) DMEM (Dulbecco's Modified Eagle Medium), IMDM (Iscove's Modified Dulbecco's Medium), RPMI-1640 and Ham's F-10 and F-12. The dosing of the cell may include any means of introducing the diluted mixture to the cell. The method will result in cellular uptake of the RNA and reduce cellular mRNA and/or protein expression for siRNA or antisense oligonucleotide delivery, or increased or unique protein expression for mRNA or plasmid DNA delivery. Preferably, the disclosed methods for siRNA may reduce mRNA and/or protein levels related by at least 50%. The delivery of mRNA or pDNA would conversely result in the expression of the encoded protein. The methods are not cytotoxic, preferably with cell viabilities equal to or exceeding 40% after 48 hours, or more preferably, cell viabilities equal to or exceeding 60% after 48 hours.

### IX. METHODS OF TREATMENT

Disclosed is a method of treating disease caused by protein expression, comprising: (a) mixing oligonucleotides with one or more amino acid derivatives as disclosed above and of the general structure disclosed in FIG. 49; (b) diluting the mixture into a medium; and (c) delivering the diluted mixture to a tissue of a subject, thereby reducing harmful protein expression or increasing beneficial protein expression. Preferably, the methods may be used to treat mammals, or more preferably, a human subject.

The disclosed methods may be used to deliver various types of therapeutic RNA or DNA to the patient's cells, including mRNA, siRNA (and its variations), miRNA, short hairpin RNA (shRNA), and/or other oligonucleotides, including plasmid DNA. Any type of cell culture medium created to support cellular growth may be used in the method, including natural media, such as biological fluids, including serum, plasma, and lymph fluids, and tissue extracts, and synthetic media such as MEM (Minimum Essential Medium) DMEM (Dulbecco's Modified Eagle Medium), IMDM (Iscove's Modified Dulbecco's Medium), RPMI-1640 and Ham's F-10 and F-12.

The administration to a patient (delivering the mixture to the patient's tissue) may occur through any method that will effectively deliver the therapeutic RNA into the target cells, for example, the cytosol (in the case of siRNA) or the nucleus (shRNA). The method of administration will depend on the targeted organs. Methods include locoregional delivery, such intranasal inhalation, and systemic delivery, such as intravenous injection. Potential methods of administration are outlined in Wang J, Lu Z, Wientjes M G, Au J L. Delivery of siRNA therapeutics: barriers and carriers. AAPS J. 2010 December; 12(4):492-503. doi: 10.1208/s12248-010-9210-4; and Tatiparti K, Sau S, Kashaw S K, Iyer A K. siRNA Delivery Strategies: A Comprehensive Review of Recent Developments. Nanomaterials (Basel). 2017 Apr. 5; 7(4):77. doi: 10.3390/nano7040077.

The method of treatment may be used to cure any condition caused by faulty protein expression. For example, mRNA, a type of ribonucleic acid that copies instructions for making proteins from DNA, transfers the instruction from inside each cell's nucleus to the protein factory in the surrounding cytoplasm. If the DNA is mutated, faulty mRNA can result. That mRNA, in turn, can instruct cells to make too many proteins or ones that do not work correctly, causing disease. The method of siRNA treatment disclosed herein should reduce the expression of proteins, including through mRNA degradation and posttranscription gene silencing. The treatment method will result in cellular uptake of the RNA and reduce cellular mRNA and/or protein expression. Preferably, the disclosed methods may reduce mRNA and/or protein levels by at least 50%. Conversely, the delivery of mRNA or plasmid DNA would result in expression of the encoded protein.

Many diseases are caused by aberrant protein expression or the expression of proteins that are modified to alter normal function. Such diseases include cancer, Huntington's Disease, coronaviruses or other viruses, Alzheimer's disease, cystic fibrosis and acute respiratory distress syndrome (ARDS).

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1-92. (canceled)

93. A nanoparticle complex comprising: short-interfering RNA (siRNA) and one or more amino acid derivatives of Formula I, II, III, IV, V, VI, or VII: 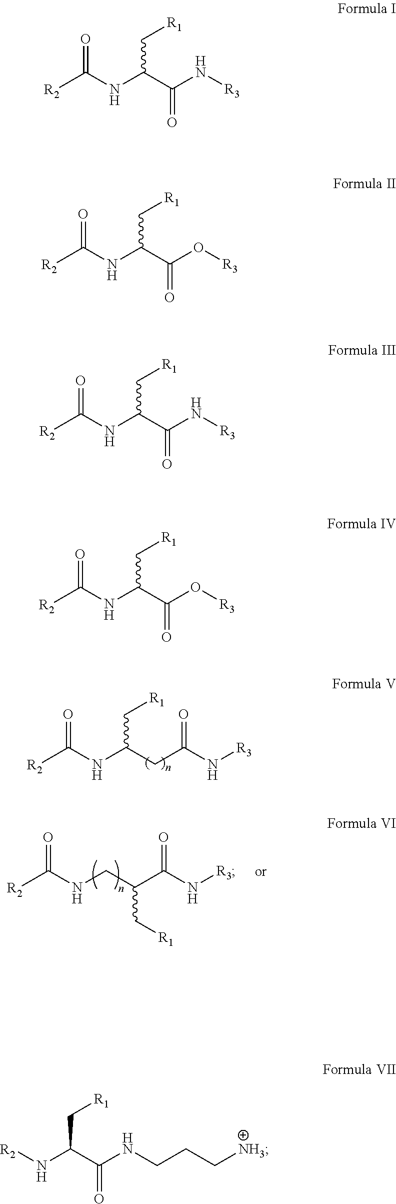
wherein R₁ is an amino acid side chain or, where the amino acid derivative is of Formula VII, 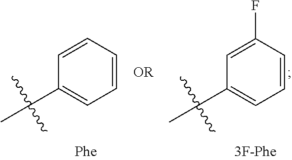 [SMILES: *c1ccccc1 ; *c1cccc(F)c1]
R₂ is selected from the group consisting of: 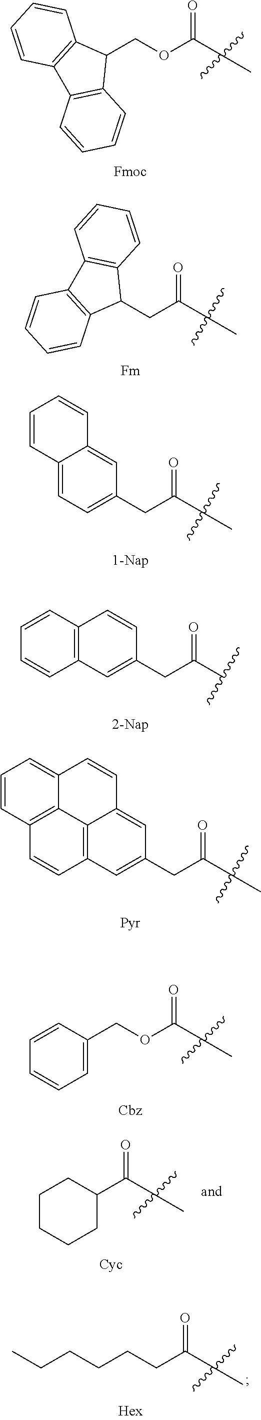
R₃ is selected from the group consisting of: 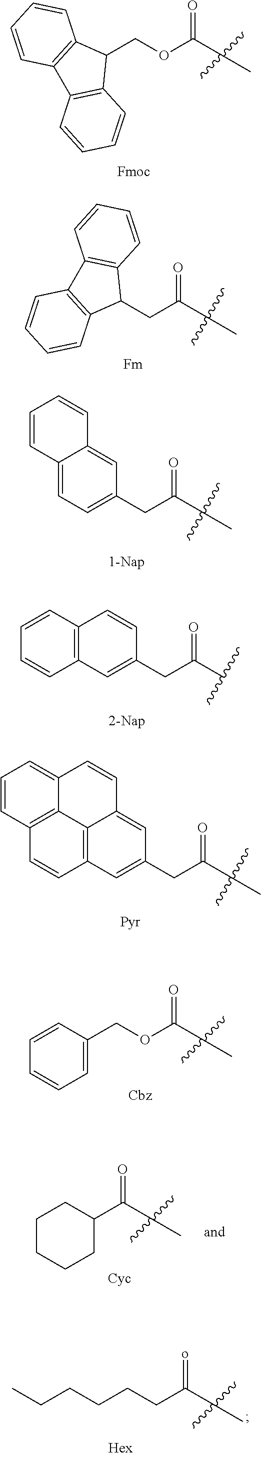
and n is an integer from 1 to 4.

94. The nanoparticle complex of claim 93, wherein the nanoparticles are primarily spherical or micelle-like.

95. The nanoparticle complex of claim 93, wherein at least one of the amino acid derivatives is an amino acid derivative of Formula VII, and wherein R₂ is

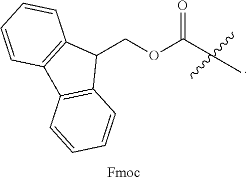 [SMILES: *C(=O)OCC1c2ccccc2-c2ccccc21]

96. The nanoparticle complex of claim 93, wherein R₁ is 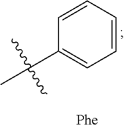 [SMILES: *c1ccccc1]
and R₂ is not

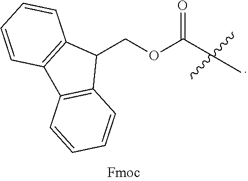 [SMILES: *C(=O)OCC1c2ccccc2-c2ccccc21]

97. The nanoparticle complex of claim 93, wherein R₂ and/or R₃ is selected from the group consisting of:

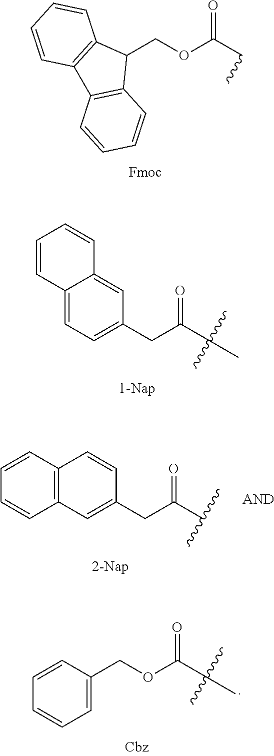

98. A method of delivering RNA or DNA, including chemically-modified derivatives of RNA or DNA, into a cell, the method comprising:
a. Mixing the RNA or DNA with one or more amino acid derivatives of Formula I, II, III, IV, V, VI, or VII: 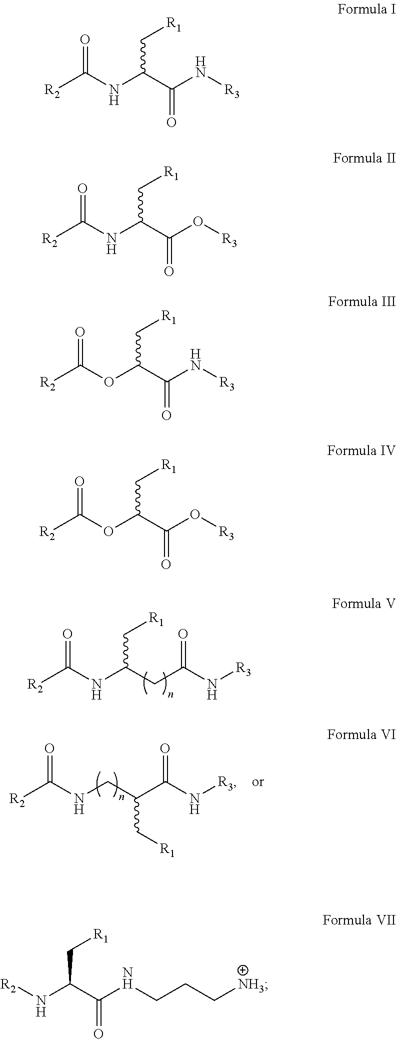
wherein R₁ is an amino acid side chain or, where the amino acid derivative is of Formula VII, 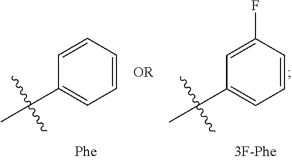 [SMILES: *c1ccccc1 ; *c1cccc(F)c1]
R₂ is selected from the group consisting of: 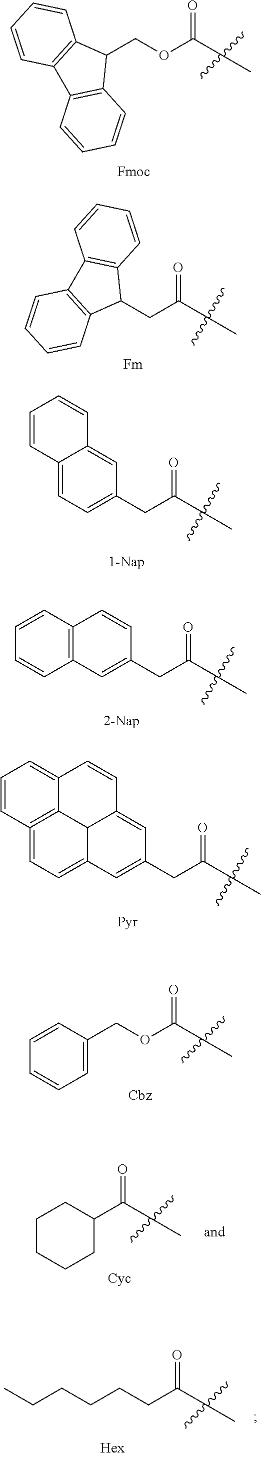
R₃ is selected from the group consisting of 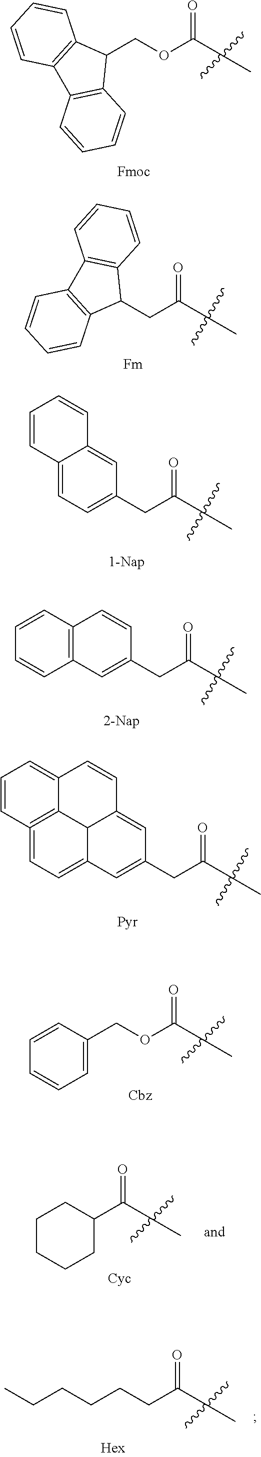
and n is an integer from 1 to 4;
b. Diluting the mixture into a cell culture medium; and
c. Dosing the cell with the diluted mixture.

99. The method of claim 98, where the RNA is small interfering RNA (siRNA).

100. The method of claim 98, where the method reduces messenger RNA (mRNA) levels and/or protein levels related to a specific gene or genes.

101. The method of claim 98, wherein at least one amino acid derivative is an amino acid derivative of Formula VII, and wherein R₂ is

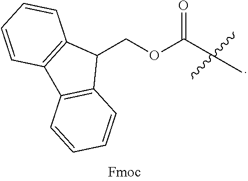 [SMILES: *C(=O)OCC1c2ccccc2-c2ccccc21]

102. The method of claim 98, wherein R₁ is 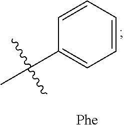 [SMILES: *c1ccccc1]
and R₂ is not

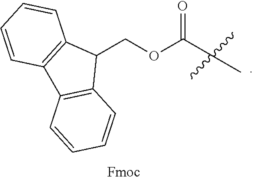 [SMILES: *C(=O)OCC1c2ccccc2-c2ccccc21]

103. The method of claim 98, wherein R₂ and/or R₃ is selected from the group consisting of:

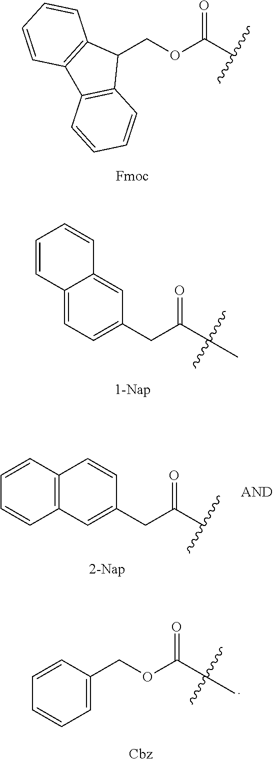 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]

104. The method of claim 98, wherein the mixture forms a nanoparticle complex and the nanoparticles are primarily spherical or micelle-like.

105. The method of claim 98, wherein the method treats disease caused by expression of excessive or dysfunctional proteins.

106. The method of claim 105, wherein the disease is cancer, Huntington's Disease, a coronavirus, Alzheimer's disease, cystic fibrosis or Acute Respiratory Distress Syndrome (ARDS).

107. The method of claim 106, wherein the disease is lung cancer.

108. A nanoparticle complex comprising: therapeutic RNA or DNA and one or more amino acid derivatives of Formula I, II, III, IV, V, VI or VII: 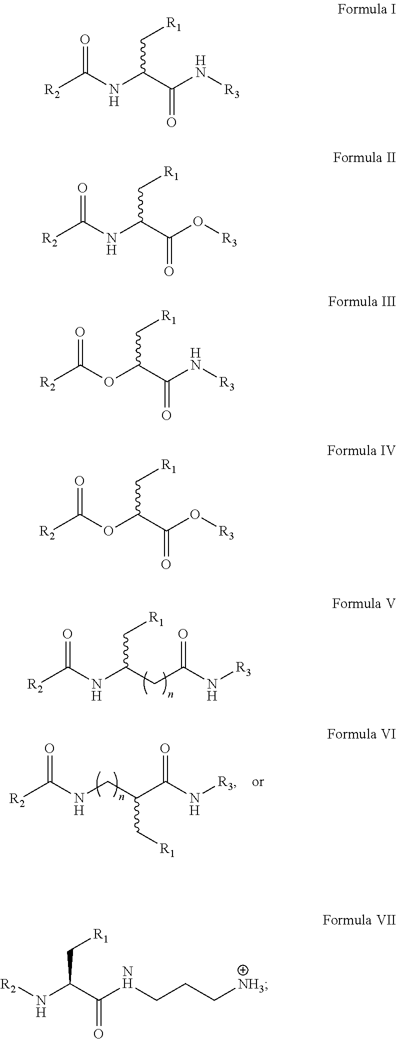
wherein R₁ is an amino acid side chain or, where the amino acid derivative is of Formula VII, 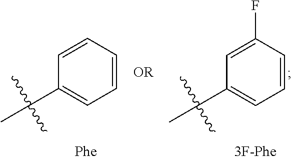 [SMILES: *c1ccccc1 ; *c1cccc(F)c1]
R₂ is selected from the group consisting of: 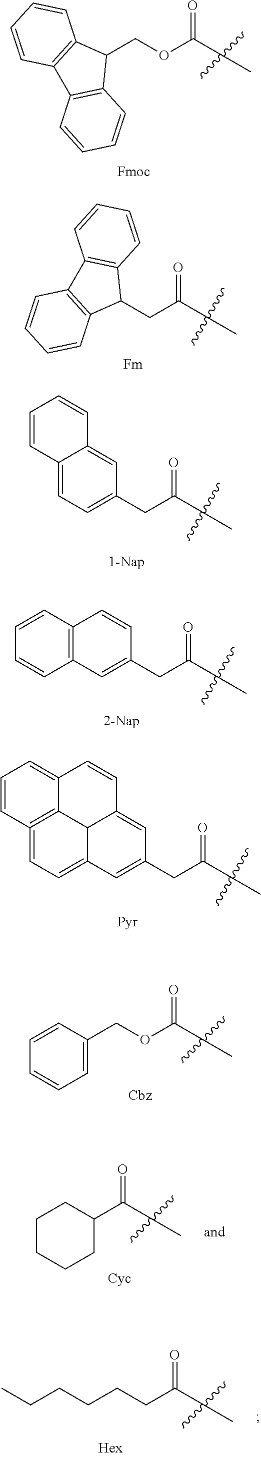
R₃ is selected from the group consisting of: 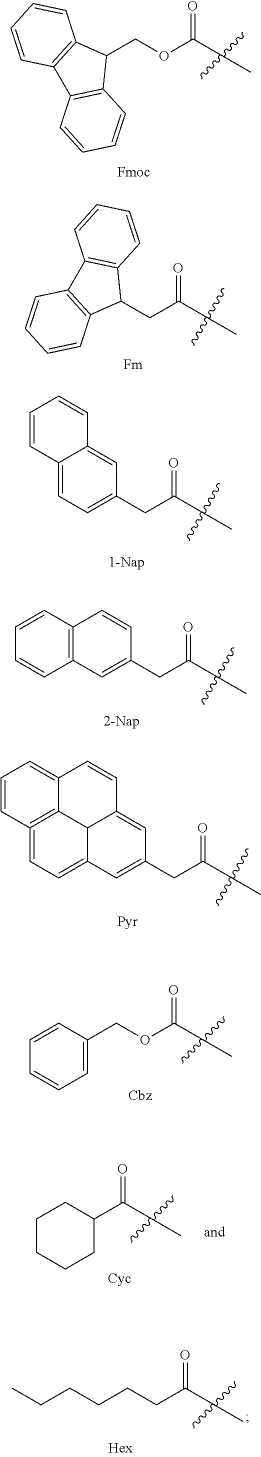
n is an integer from 1 to 4.

109. The nanoparticle complex of claim 108, wherein the nanoparticles are spherical or micelle-like.

110. The nanoparticle complex of claim 108, wherein the RNA or DNA comprises antisense oligonucleotides, messenger RNA (mRNA), short interfering RNA (siRNA) or plasmid DNA (pDNA).

111. The nanoparticle complex of claim 108, wherein at least one amino acid derivative is an amino acid derivative of Formula VII, and wherein R₂ is

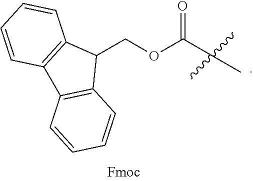 [SMILES: *C(=O)OCC1c2ccccc2-c2ccccc21]

112. The nanoparticle complex of claim 108, wherein R₁ is 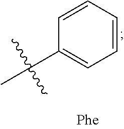 [SMILES: *c1ccccc1]
and R₂ is not

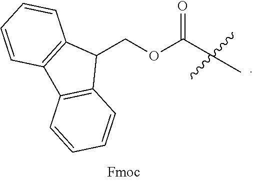 [SMILES: *C(=O)OCC1c2ccccc2-c2ccccc21]

113. The nanoparticle complex of claim 108, wherein R₂ and/or R₃ is selected from the group consisting of: 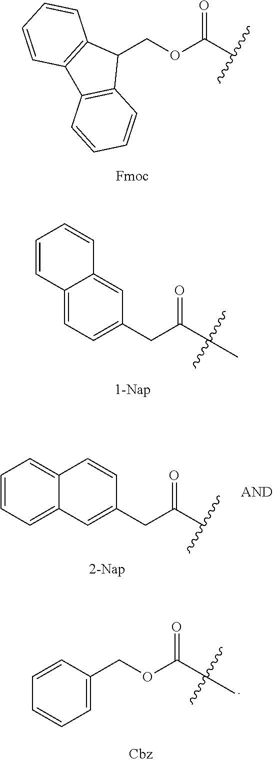 [SMILES: *C(=O)OCC1c2ccccc2-c2ccccc21 ; *C(=O)Cc1ccc2ccccc2c1 ; *C(=O)Cc1ccc2ccccc2c1 ; *C(=O)OCc1ccccc1]
